(19) United States
(12) Patent Application Publication
(10) Pub. No.: US 2026/0146064 A1
(43) Pub. Date: May 28, 2026

(54) PH-SENSITIVE MEMBRANE DISRUPTIVE PEPTIDES AND USE THEREOF

(71) Applicants: SOUTH CHINA UNIVERSITY OF TECHNOLOGY, Guangzhou (CN); SUN YAT-SEN MEMORIAL HOSPITAL, SUN YAT-SEN UNIVERSITY, Guangzhou (CN)

(72) Inventors: Menghua XIONG, Guangzhou (CN); Yan BAO, Guangzhou (CN); Jihong WANG, Guangzhou (CN); Zining WU, Guangzhou (CN)

(73) Assignees: SOUTH CHINA UNIVERSITY OF TECHNOLOGY, Guangzhou (CN); SUN YAT-SEN MEMORIAL HOSPITAL, SUN YAT-SEN UNIVERSITY, Guangzhou (CN)

(21) Appl. No.: 19/407,028

(22) Filed: Dec. 3, 2025

Related U.S. Application Data
(63) Continuation of application No. PCT/CN2024/099895, filed on Jun. 18, 2024.

(30) Foreign Application Priority Data
Jun. 19, 2023 (CN) 202310727046.2

(51) Int. Cl. C07K 14/00; A61K 38/00

(52) U.S. Cl. CPC C07K 14/001; A61K 38/00

(57) ABSTRACT

Provided is a pH-sensitive membrane disruptive peptide having a structure as shown in formula (I) or a stereoisomer thereof or a pharmaceutically acceptable salt thereof, and a use thereof. The macromolecular material is hydrophobic and electrically neutral at normal physiological pH, and can self-assemble into nanoparticles with tight assembly and weak interaction with cell membranes. Under a slightly acidic pH condition, the macromolecular material can be protonated to form an amphiphilic structure composed of a hydrophobic domain and a cationic domain, which has strong interaction with the cell membranes and strong membrane disruptive activity, and thus can efficiently and highly selectively kill tumor cells.

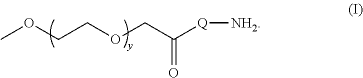

### CROSS-REFERENCE TO RELATED APPLICATIONS

This application is a Continuation of International Application No. PCT/CN2024/099895, filed on Jun. 18, 2024, which claims priority to Chinese Patent Application No. 202310727046.2, filed on Jun. 19, 2023, the entire contents of each of which are hereby incorporated by reference.

### TECHNICAL FIELD

The present disclosure generally relates to the field of macromolecular materials and pharmaceutical technology, and in particular to a pH-sensitive membrane disruptive peptide and a use thereof.

### BACKGROUND

Some amphiphilic macromolecules containing cationic groups can kill tumor cells, bacteria, and other pathogens by disrupting cell membranes. This membrane-disruptive killing mechanism, which is independent of metabolic pathways, exhibits broad-spectrum activity and reduces risk of drug resistance, offering great potential for the treatment of tumors and infectious diseases. The interaction between membrane disruptive macromolecular materials and cell membranes mainly involves electrostatic interactions and hydrophobic interactions. Cationic domains of the macromolecular materials first bind to a surface of negatively charged cell membranes through the electrostatic interactions, after which hydrophobic domains of the macromolecular materials insert into the lipid layers of the cell membranes, leading to an irreversible membrane damage and thereby inducing cell death. An amphiphilic conformation composed of the cationic groups and hydrophobic groups is not only a key structure for an interaction between macromolecular materials and the cell membranes, but also a major cause of cytotoxicity to normal tissue cells. A structural ratio of the cationic groups and the hydrophobic groups plays a crucial role in modulating the interaction with the cell membranes. In general, pure cationic macromolecules readily bind to the cell membranes but have limited ability to insert into the cell membrane, while hydrophobic polymers exhibit poor binding to a cell membrane surface and thus cannot effectively disrupt the cell membrane. Therefore, it is of great significance to design macromolecule materials that exhibit either cationic or hydrophobic structures in normal tissues, but undergo transformation into the amphiphilic conformations composed of the cationic domains and the hydrophobic domains at lesion sites, thereby addressing an issue of high toxicity of the membrane disruptive macromolecule materials.

The research team of the present disclosure previously prepared polymethacrylate macromolecule materials containing tertiary amine side chains with membrane disruptive activity. Protonation of the tertiary amine groups on the side chains enables controlled activation of the membrane disruptive activity at different pH levels. Furthermore, by copolymerizing to introduce hydrophobic segments, it is possible to achieve low hemolysis and cytotoxicity at pH=7.4, while the membrane disruptive activity is precisely activated in a slightly acidic environment, causing irreversible damage to membranes of the tumor cells and achieving highly selective killing of the tumor cells. However, a polymethacrylate backbone is difficult to degrade, and a long-term accumulation in vivo may lead to toxicity.

### SUMMARY

In view of the above, the present disclosure provides a pH-sensitive membrane disruptive peptide material containing tertiary amines and hydrophobic groups in its side groups. This macromolecular material is hydrophobic and electrically neutral under a normal physiological pH and has a weak interaction with cell membranes. Under a slightly acidic pH condition, the macromolecular material can be protonated to form an amphiphilic structure composed of hydrophobic domains and cationic domains. This structure has a strong interaction with the cell membranes and exhibits strong membrane disruptive activity, thereby enabling efficient and highly selective killing of tumor cells. In addition, the polypeptide may be synthesized using a solid-phase synthesis process, and the obtained polypeptide material maintains a fixed amino acid sequence and number. The present disclosure includes following technical solutions.

One or more embodiments of the present disclosure provide a membrane disruptive peptide having a structure as shown in formula (I) or a stereoisomer thereof or a pharmaceutically acceptable salt thereof:

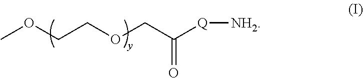

wherein Q is a polypeptide group formed by arranging n A and m B in any sequence;
B is a residue formed by removing one amino hydrogen and one hydroxyl group from an amino acid having a hydrophobic side chain;
A is a residue formed by removing one amino hydrogen and one hydroxyl group from a lysine having a modifying group, and has a structure:

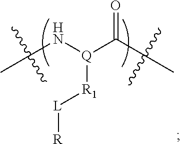

R is selected from the group consisting of: —R₃—N(R₄R₅), —R₃—R′, and

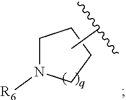

R′ is

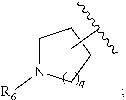

L is selected from the group consisting of —NH—C(═O)O—, —NH—C(═O)—, —C(═O)—NH—, and —C(═O)—O—;
R₁ is an alkylene;
R₃ is selected from the group consisting of an alkylene, and a C₆-C₁₄ aryl-substituted alkylene;
R₄ and R₅ are each independently selected from the group consisting of an alkyl, a C₆-C₁₄ aryl-substituted alkyl, or R₄, R₅, and a nitrogen atom to which R₄ and R₅ are connected form a heterocycloalkyl;
R₆ is selected from the group consisting of a C₁-C₁₅ alkyl, a C₆-C₁₄ aryl, and a C₆-C₁₄ aryl-substituted C₁-C₁₅ alkyl;
y is selected from: 2-150;
n+m is greater than 0, and n is not 0; and
q is selected from: 0, 1, 2, 3, and 4.

In some embodiments, the membrane disruptive peptide has a structure as shown in formula (II):

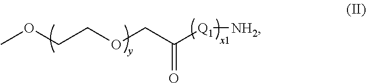

wherein Q₁ is a polypeptide group formed by arranging n₁ A and m₁ B in any sequence;
n₁+m₁ is greater than 0, and n₁ is not 0;
x₁ is greater than 0; and
a product of n₁ and x₁ is n; and a product of m₁ and x₁ is m.

In some embodiments, the membrane disruptive peptide has a structure as shown in formula (III):

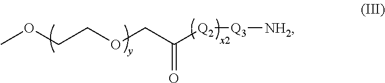

wherein Q₂ is a polypeptide group formed by arranging n₂ A and m₂ B in any sequence;
Q₃ is a polypeptide group formed by arranging n₃ A and m₃ B in any sequence;
n₂+n₃+m₂+m₃ is greater than 0, and n₂+n₃ is not 0; and
a product of n₂ and x₂ plus n₃ is n, and a product of m₂ and x₂ plus m₃ is m.

One or more embodiments of the present disclosure provide a membrane disruptive peptide nanoparticle, including following technical solutions.

The membrane disruptive peptide nanoparticle is formed by self-assembly of the above-mentioned membrane disruptive peptide in an aqueous medium.

One or more embodiments of the present disclosure provide a method for preparing a membrane disruptive peptide nanoparticle, including following technical solutions.

The method for preparing a membrane disruptive peptide nanoparticle, including following operations: dissolving the membrane disruptive peptide in an organic solvent or a hydrochloric acid solution with a pH of 1.5-2.5 to obtain a solution, then dropwise adding the obtained solution into water under a stirring state, continuing stirring, and dialyzing to remove the solvent, thereby obtaining the membrane disruptive peptide nanoparticle.

One or more embodiments of the present disclosure provide a use of the membrane disruptive peptide or the membrane disruptive peptide nanoparticle, including following technical solutions.

A use of the membrane disruptive peptide or the stereoisomer thereof or the pharmaceutically acceptable salt thereof in preparation of a medicament for preventing and/or treating a tumor is provided.

A use of the membrane disruptive peptide nanoparticle in preparation of a medicament for preventing and/or treating a tumor is provided.

One or more embodiments of the present disclosure provide a medicament for preventing and/or treating a tumor, including following technical solutions.

The medicament for preventing and/or treating a tumor is prepared from an active ingredient and a pharmaceutically acceptable excipient; the active ingredient includes the membrane disruptive peptide or the stereoisomer thereof or the pharmaceutically acceptable salt thereof, and/or the membrane disruptive peptide nanoparticle.

One or more embodiments of the present disclosure provide a method for preventing and/or treating a tumor, including following technical solutions.

The method for preventing and/or treating a tumor comprises: administering a safe and effective amount of the membrane disruptive peptide or the stereoisomer thereof or the pharmaceutically acceptable salt thereof, and/or administering a safe and effective amount of the membrane disruptive peptide nanoparticle; and/or administering a safe and effective amount of the medicament to a patient.

### BRIEF DESCRIPTION OF THE DRAWING

The patent or application file contains at least one drawing executed in color. Copies of this patent or patent application publication with color drawing(s) will be provided by the Office upon request and payment of the necessary fee.

The present disclosure will be further illustrated by way of exemplary embodiments, which will be described in detail with reference to accompanying drawings. These embodiments are not limiting, wherein:

FIG. 1 is a mass spectrum of (KW)₅K according to some embodiments of the present disclosure.

FIG. 2 is a mass spectrum of (KW)₆K according to some embodiments of the present disclosure.

FIG. 3 is a mass spectrum of (KW)₇K according to some embodiments of the present disclosure.

FIG. 4 is a mass spectrum of (KW)₈K according to some embodiments of the present disclosure.

FIG. 5 is a mass spectrum of (KWWK)₂KKW according to some embodiments of the present disclosure.

FIG. 6 is a mass spectrum of (KWWK)₃K according to some embodiments of the present disclosure.

FIG. 7 is a mass spectrum of (KWWK)₃KKW according to some embodiments of the present disclosure.

FIG. 8 is a mass spectrum of (KWWK)₄K according to some embodiments of the present disclosure.

FIG. 9 is a mass spectrum of (KWK)₃ according to some embodiments of the present disclosure.

FIG. 10 is a mass spectrum of (KWK)₄ according to some embodiments of the present disclosure.

FIG. 11 is a mass spectrum of (KWK)₅ according to some embodiments of the present disclosure.

FIG. 12 is a mass spectrum of (KWK)₆ according to some embodiments of the present disclosure.

FIG. 13 is a mass spectrum of (KWK)₇ according to some embodiments of the present disclosure.

FIG. 14 is a mass spectrum of (KWKK)₃ according to some embodiments of the present disclosure.

FIG. 15 is a mass spectrum of (KWKK)₄ according to some embodiments of the present disclosure.

FIG. 16 is a mass spectrum of (KWKK)₅ according to some embodiments of the present disclosure.

FIG. 17 is a mass spectrum of (KWKK)₇ according to some embodiments of the present disclosure.

FIG. 18 is a mass spectrum of (KKWKK)₃ according to some embodiments of the present disclosure.

FIG. 19 is a mass spectrum of (KKWKK)₅ according to some embodiments of the present disclosure.

FIG. 20 is a proton nuclear magnetic resonance spectrum of mPEG₄₅-(K₁W)ₓK₁ according to some embodiments of the present disclosure.

FIG. 21 is a proton nuclear magnetic resonance spectrum of mPEG₄₅-(K₁WWK₁)ₓK₁ according to some embodiments of the present disclosure.

FIG. 22 is a proton nuclear magnetic resonance spectrum of mPEG₄₅-(K₁WWK₁)ₓK₁K₁W according to some embodiments of the present disclosure.

FIG. 23 is a proton nuclear magnetic resonance spectrum of mPEG₄₅-(K₁WK₁)ₓ according to some embodiments of the present disclosure.

FIG. 24 is a proton nuclear magnetic resonance spectrum of mPEG₄₅-(K₁WK₁K₁)ₓ according to some embodiments of the present disclosure.

FIG. 25 is a proton nuclear magnetic resonance spectrum of mPEG₄₅-(K₁K₁WK₁K₁)ₓ according to some embodiments of the present disclosure.

FIG. 26 is a schematic diagram illustrating a protonation degree of mPEG₄₅-(K₁)ₙ(W)ₘvarying with pH according to some embodiments of the present disclosure.

FIG. 27 is a schematic diagram illustrating cytotoxicity of mPEG₄₅-(K₁)ₙ(W)ₘ against MC38 cells at pH 7.4 according to some embodiments of the present disclosure.

FIG. 28 is a schematic diagram illustrating cytotoxicity of mPEG₄₅-(K₁)ₙ(W)ₘ against MC38 cells at pH 6.8 according to some embodiments of the present disclosure.

FIG. 29 is a schematic diagram illustrating a maximum tolerated dose (MTD) of mPEG₄₅-(K₁)ₙ(W)ₘ for ICR mice according to some embodiments of the present disclosure.

FIG. 30 is a schematic diagram illustrating images of mPEG₄₅-(K₁)ₙ(W)ₘ incubated with Panc02mCherry/GFP cells at different pH values obtained through a high-content imaging system according to some embodiments of the present disclosure.

FIG. 31 is a schematic diagram illustrating a tumor growth curve after treatment of an MC38 subcutaneous tumor model with a sequence-defined polypeptide according to some embodiments of the present disclosure.

FIG. 32 is a schematic diagram illustrating a change in body weight of mice after treatment of an MC38 subcutaneous tumor model with a sequence-defined polypeptide according to some embodiments of the present disclosure.

FIG. 33 is a photograph of a tumor after treatment of an MC38 subcutaneous tumor model with a sequence-defined polypeptide according to some embodiments of the present disclosure.

FIG. 34 is an image of mPEG₄₅-(K₁WK₁)₅ incubated with Panc02mCherry/GFP cells at pH 6.8 for different times according to some embodiments of the present disclosure.

FIG. 35 is an image of mPEG₄₅-(K₁WK₁)₅ incubated with MC38 cells at pH 6.8 for different times in a presence of Annexin V-FITC and propidium iodide (PI) according to some embodiments of the present disclosure.

FIG. 36 is a proton nuclear magnetic resonance spectrum of mPEG₄₅-(K₂K₂WK₂K₂)₅ according to some embodiments of the present disclosure.

FIG. 37 is a proton nuclear magnetic resonance spectrum of mPEG₄₅-(K₃K₃WK₃K₃)₅ according to some embodiments of the present disclosure.

FIG. 38 is a proton nuclear magnetic resonance spectrum of mPEG₄₅-(K₄K₄WK₄K₄)₅ according to some embodiments of the present disclosure.

FIG. 39 is a proton nuclear magnetic resonance spectrum of mPEG₄₅-(K₅K₅WK₅K₅)₅ according to some embodiments of the present disclosure.

FIG. 40 is a proton nuclear magnetic resonance spectrum of mPEG₄₅-(K₆K₆WK₆K₂)₅ according to some embodiments of the present disclosure.

FIG. 41 is a proton nuclear magnetic resonance spectrum of mPEG₄₅-(K₇K₇WK₇K₇)₅ according to some embodiments of the present disclosure.

FIG. 42 is a proton nuclear magnetic resonance spectrum of mPEG₄₅-(K₈K₈WK₈K₈)₅ according to some embodiments of the present disclosure.

FIG. 43 is a schematic diagram illustrating a protonation degree of polypeptides modified with different tertiary amines on mPEG₄₅-(KKWKK)₅ varying with pH according to some embodiments of the present disclosure.

FIG. 44 is a schematic diagram illustrating cytotoxicity of polypeptides modified with different tertiary amines on mPEG₄₅-(KKWKK)₅ against MC38 cells at pH 7.4 and 6.8 according to some embodiments of the present disclosure.

FIG. 45 is a proton nuclear magnetic resonance spectrum of mPEGy-(K₁K₁WK₁K₁)₅ with PEG of different lengths according to some embodiments of the present disclosure.

FIG. 46 is a schematic diagram illustrating a protonation degree of mPEGy-(K₁K₁WK₁K₁)₅ with PEG of different lengths varying with pH according to some embodiments of the present disclosure.

FIG. 47 is a schematic diagram illustrating cytotoxicity of mPEGy-(K₁K₁WK₁K₁)₅ with PEG of different lengths against MC38 cells at pH 7.4 and pH 6.8 according to some embodiments of the present disclosure.

FIG. 48 is a mass spectrum of (KKKKW)₅ according to some embodiments of the present disclosure.

FIG. 49 is a mass spectrum of (K)₅(W)₂(K)₁₀(W)₃(K)₅ according to some embodiments of the present disclosure.

FIG. 50 is a mass spectrum of (K)₅(W)₂(K)₅W(K)₅(W)₂(K)₅ according to some embodiments of the present disclosure.

FIG. 51 is a proton nuclear magnetic resonance spectrum of polypeptides with different arrangements constructed from mPEG₄₅-(K₁K₁WK₁K₁)₅ according to some embodiments of the present disclosure.

FIG. 52 is a schematic diagram illustrating a protonation degree of polypeptides with different arrangements constructed from mPEG₄₅-(K₁K₁WK₁K₁)₅ varying with pH according to some embodiments of the present disclosure.

FIG. 53 is a schematic diagram illustrating cytotoxicity of polypeptides with different arrangements constructed from mPEG₄₅-(K₁K₁WK₁K₁)₅ against MC38 cells at pH 7.4 and pH 6.8 according to some embodiments of the present disclosure.

FIG. 54 is a mass spectrum of (KKKFKKK)₂K according to some embodiments of the present disclosure.

FIG. 55 is a mass spectrum of (KKFKK)₃ according to some embodiments of the present disclosure.

FIG. 56 is a mass spectrum of (KFK)₄KKK according to some embodiments of the present disclosure.

FIG. 57 is a mass spectrum of (KFK)₅ according to some embodiments of the present disclosure.

FIG. 58 is a proton nuclear magnetic resonance spectrum of mPEG₄₅-(K₁)ₙ(F)ₘ according to some embodiments of the present disclosure.

FIG. 59 is a proton nuclear magnetic resonance spectrum of mPEG₄₅-(K₉)ₙ(F)ₘ according to some embodiments of the present disclosure.

FIG. 60 is a schematic diagram illustrating a protonation degree of mPEG₄₅-(K₁)ₙ(F)ₘ varying with pH according to some embodiments of the present disclosure.

FIG. 61 is a schematic diagram illustrating a protonation degree of mPEG₄₅-(K₉)ₙ(F)ₘ varying with pH according to some embodiments of the present disclosure.

FIG. 62 is a schematic diagram illustrating cytotoxicity of mPEG₄₅-(K₁)ₙ(F)ₘ against MC38 cells at pH 7.4 and pH 6.8 according to some embodiments of the present disclosure.

FIG. 63 is a schematic diagram illustrating cytotoxicity of mPEG₄₅-(K₉)ₙ(F)ₘ against MC38 cells at pH 7.4 and pH 6.8 according to some embodiments of the present disclosure.

### DETAILED DESCRIPTION

For experimental manners without specified conditions in following embodiments of the present disclosure, the methods are generally performed under conventional conditions or under conditions recommended by the manufacturer. Various commonly used chemical reagents in the embodiments are commercially available products.

Unless otherwise defined, all technical and scientific terms used in the present disclosure have same meanings as commonly understood by a person of ordinary skill in the art to which the present disclosure belongs. The terms used in the present disclosure are for a purpose of describing specific embodiments only and are not intended to limit the present disclosure.

The terms “include” and “have” in the present disclosure and any variations thereof are intended to cover non-exclusive inclusion. For example, a process, method, apparatus, product, or device that includes a series of steps or modules is not limited to the listed steps or modules, but optionally further includes steps that are not listed, or optionally further includes other steps inherent to the process, method, product, or device.

“A plurality of” as mentioned in the present disclosure refers to two or more. “And/or” describes an association relationship between associated objects and indicates that three relationships may exist. For example, A and/or B may indicate: A exists alone, A and B exist simultaneously, and B exists alone. The character “/” generally indicates that associated objects preceding and following the character are related in an “or” relationship.

In compounds described in the present disclosure, when any variable (e.g., R₁, R₂, etc.) appears more than once in any component, a definition of each occurrence is independent of a definition of every other occurrence. Similarly, combinations of substituents and variables are allowed as long as the combinations result in stable compounds. A line extending from a substituent into a ring system indicates that the referenced bond may be connected to any substitutable atom of the ring. If the ring system is polycyclic system, it means that the bond is only connected to any appropriate carbon atom of an adjacent ring. It should be understood that a person of ordinary skill in the art may select substituents and substitution patterns of the compounds of the present disclosure to provide chemically stable compounds that can be readily synthesized from readily available starting materials using techniques known in the art and manners described below. If a substituent is itself substituted by more than one group, it should be understood that the groups may be on a same carbon atom or on different carbon atoms, provided that a structure is stable. In the present disclosure, “*” and “/” in a chemical group or chemical structure both represent bond positions.

The term “alkyl” refers to saturated aliphatic hydrocarbon groups having a specified count of carbon atoms, including both straight-chain forms and branched-chain forms. For example, the definition “C₁-C₆” in “C₁-C₆ alkyl” includes groups having 1, 2, 3, 4, 5, or 6 carbon atoms arranged in a straight or branched chain. For example, the “C₁-C₆ alkyl” specifically includes methyl, ethyl, n-propyl, isopropyl, n-butyl, tert-butyl, isobutyl, pentyl, and hexyl.

The term “alkylene” refers to a group having one less hydrogen than the “alkyl”, such as —CH₂—, —CH₂CH₂—, —CH₂CH₂CH₂—, —CH₂CH₂CH₂CH₂—, etc.

The term “heterocycloalkyl” refers to a saturated monocyclic cyclic substituent in which one or more ring atoms are heteroatoms selected from N, O, or S(O)ₘ (where m is an integer from 0 to 2), and remaining ring atoms are carbon atoms, such as piperidinyl, pyrrolidinyl, etc.

The term “fixed sequence” or “sequence-defined” refers to a defined arrangement of amino acids within a polypeptide chain.

One or more embodiments of the present disclosure provide a membrane disruptive peptide having a structure as shown in formula (I) or a stereoisomer thereof or a pharmaceutically acceptable salt thereof:

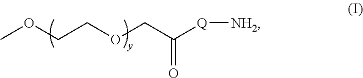

wherein Q is a polypeptide group formed by arranging n A and m B in any sequence;
B is a residue formed by removing one amino hydrogen and one hydroxyl group from an amino acid having a hydrophobic side chain;
A is a residue formed by removing one amino hydrogen and one hydroxyl group from a lysine having a modifying group, and has a structure:

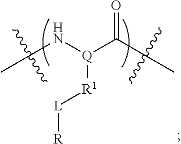

R is selected from the group consisting of: —R₃—N(R₄R₅), —R₃—R′, and

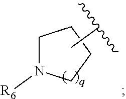

R′ is

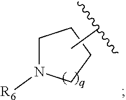

L is selected from the group consisting of: —NH—C(═O)O—, —NH—C(═O)—, —C(═O)—NH—, and —C(═O)—O—;
R₁ an alkylene;
R₃ is selected from the group consisting of: an alkylene and a C₆-C₁₄ aryl-substituted alkylene;
R₄ and R₅ are each independently selected from the group consisting of: an alkyl and a C₆-C₁₄ aryl-substituted alkyl, or R₄, R₅, and the nitrogen atom to which R₄ and R₅ are connected form a heterocycloalkyl;
R₆ is selected from the group consisting of: a C₁-C₁₅ alkyl, a C₆-C₁₄ aryl, and a C₆-C₁₄ aryl-substituted C₁-C₁₅ alkyl;
y is selected from: 2-150;
n+m is greater than 0, and n is not 0; and
q is selected from: 0, 1, 2, 3, and 4.

In some embodiments, the membrane disruptive peptide has a structure as shown in formula (II):

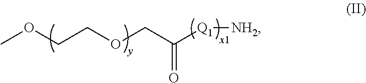

wherein Q₁ is a polypeptide group formed by arranging n₁ A and m₁ B in any sequence;
n₁+m₁ is greater than 0, and n₁ is not 0;
x₁ is greater than 0; and
the product of n₁ and x₁ is n; and the product of m₁ and x₁ is m.

In some embodiments, the membrane disruptive peptide has a structure as shown in formula (III):

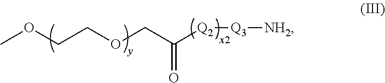

wherein Q₂ is a polypeptide group formed by arranging n₂ A and m₂ B in any sequence;
Q₃ is a polypeptide group formed by arranging n₃ A and m₃ B in any sequence;
n₂+n₃+m₂+m₃ is greater than 0, and n₂+n₃ is not 0; and
the product of n₂ and x₂ plus n₃ is n, and the product of m₂ and x₂ plus m₃ is m.

In some embodiments, n, m, q, n₁, m₁, n₂, m₂, n₃, and m₃ are all integers.

In the present disclosure, Q, Q₁, Q₂, and Q₃ refer to polypeptide groups, in which the amino acids may be arranged in any determined sequence. For example, n A and m B in Q may be arranged in any sequence. As another example, when n is 2 and m is 2, Q may be arranged in the following sequences: ABAB, ABBA, AABB, BBAA, BAAB, BABA, etc. The polypeptide may be synthesized using a solid-phase synthesis manner. Therefore, sequences and counts in Q, Q₁, Q₂, and Q₃ may be kept fixed and controllable as needed.

The amino acid with the hydrophobic side chain may include: L-amino acid, tryptophan, phenylalanine, alanine, valine, leucine, isoleucine, or the like.

In some embodiments, B is:

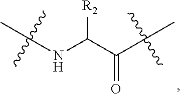

and R₂ is selected from the group consisting of: a C₁-C₁₂ alkyl, a C₆-C₁₄ aryl, a C₆-C₁₄ aryl-substituted C₁-C₁₂ alkyl, a benzyloxycarbonyl-substituted C₁-C₁₂ alkyl, and a 5-10-membered heteroaryl-substituted C₁-C₁₂ alkyl.

In some embodiments, R₂ is selected from the group consisting of: a C₁-C₅ alkyl, phenyl, naphthyl, a phenyl-substituted C₁-C₆ alkyl, a benzyloxycarbonyl-substituted C₁-C₆ alkyl, and a 5-10-membered heteroaryl-substituted C₁-C₆ alkyl.

In some embodiments, R₂ is selected from the group consisting of: a methyl, an ethyl, a n-propyl, an isopropyl, a n-butyl, an isobutyl, a pentyl, a hexyl, a heptyl, an octyl, a nonyl, a decyl, an undecyl, a dodecyl, a phenyl, a naphthyl, a benzyl, a benzyloxycarbonyl substituted ethyl, a benzopyrrole-substituted ethyl, and

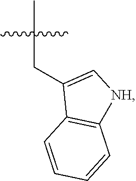 [SMILES: *Cc1c[nH]c2ccccc12]

or chiral isomers thereof.

In some embodiments, R₁ is a C₁-C₆ alkylene.

In some embodiments, R₁ is selected from the group consisting of: —(CH₂)ₚ—, wherein p is selected from: 1, 2, 3, 4, 5, and 6.

In some embodiments, A is:

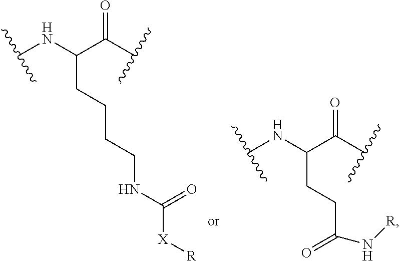

or chiral isomers thereof, wherein X is —O— or absent.

In some embodiments, R₃ is selected from the group consisting of: a C₁-C₆ alkylene and a phenyl-substituted C₁-C₆ alkylene.

In some embodiments, R₃ is selected from the group consisting of: —(CH₂)ₚ— and phenyl-substituted —(CH₂)ₚ—; wherein p is selected from: 1, 2, 3, 4, 5, and 6.

In some embodiments, R is selected from the group consisting of: —R₃—N(R₄R₅),

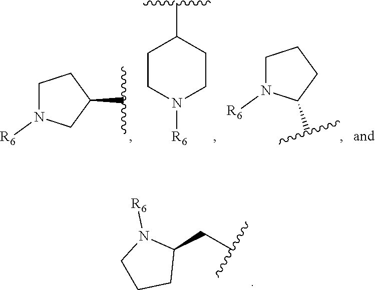

In some embodiments, R₄ and R₅ are each independently selected from the group consisting of: a C₁-C₆ alkyl, a phenyl-substituted C₁-C₆ alkyl, and a naphthyl-substituted C₁-C₆ alkyl, or R₄, R₅, and the nitrogen atom to which R₄ and R₅ are connected form a 5-10-membered heterocycloalkyl.

In some embodiments, R₄ and R₅ are each independently selected from the group consisting of: a C₁-C₄ alkyl, a phenyl-substituted C₁-C₃ alkyl, and a naphthyl-substituted C₁-C₃ alkyl, or R₄, R₅, and the nitrogen atom to which R₄ and R₅ are connected form a 5-8-membered heterocycloalkyl.

In some embodiments, R₄ and R₅ are each independently selected from the group consisting of: a C₃ alkyl, a C₄ alkyl, and a C₅ alkyl, or R₄, R₅, and the nitrogen atom to which R₄ and R₅ are connected together form a 7-membered heterocycloalkyl.

In some embodiments, R₄, R₅, and the nitrogen atom to which R₄ and R₅ are connected form a group selected from:

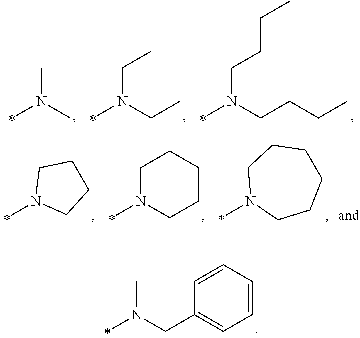

In some embodiments, R₆ is selected from the group consisting of: a C₁-C₆ alkyl, a phenyl, naphthyl, and a phenyl-substituted C₁-C₆ alkyl.

In some embodiments, R₆ is selected from the group consisting of: a C₁-C₃ alkyl, a phenyl, a naphthyl, and a benzyl.

In some embodiments, R is selected from the group consisting of: —R₃—N(R₄R₅),

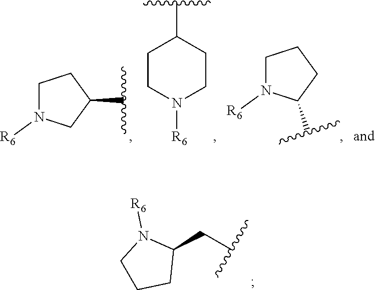

wherein R₃ is selected from the group consisting of: —CH₂—CH₂—, —CH₂—CH₂—CH₂—, —CH₂—(CH₂)₃—CH₂—, and

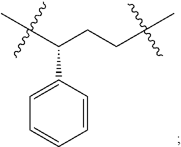 [SMILES: *CC[C@@H](*)c1ccccc1]

R₄, R₅, and the nitrogen atom to which R₄ and R₅ are connected form a group selected from:

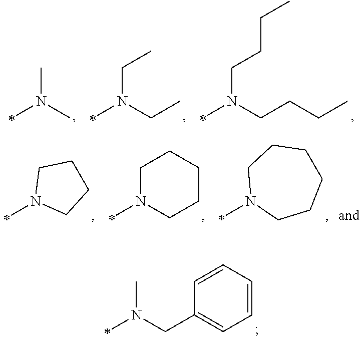

and R₆ is benzyl.

In some embodiments, R is —R₃—N(R₄R₅); wherein R₃ is —CH₂—CH₂—; R₄, R₅, and the nitrogen atom to which R₄ and R₅ are connected form a group selected from:

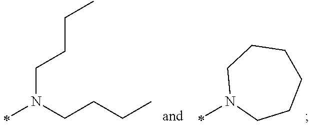

### and R₂ is

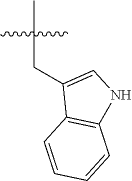 [SMILES: *Cc1c[nH]c2ccccc12]

or a chiral isomer thereof.

In some embodiments, y is selected from: 10-120. In some embodiments, y is selected from: 20-120. In some embodiments, y is selected from: 20-50, 20-25, 40-48, or 108-116. In some embodiments, y is selected from: 20, 21, 22, 23, 24, 43, 44, 45, 46, 111, 112, 113, or 114.

In some embodiments, n+m is not less than 5. In some embodiments, n+m is not less than 9. In some embodiments, n+m is not less than 12. In some embodiments, n+m is not less than 15. In some embodiments, n+m is within a range of 6-200. In some embodiments, n+m is within a range of 6-150. In some embodiments, n+m is within a range of 6-110. In some embodiments, n+m is within a range of 6-20, 5-40, or 10-30. In some embodiments, n+m is within a range of 13-30, 13-28, 13-21, or 15-25, and may be 9, 10, 11, 12, 13, 14, 15, 16, 17, 18, 19, 20, 21, 22, 23, 24, 25, 26, 27, 28, 29, or 30.

In some embodiments, n is selected from 5-30, and m is selected from 0-15 or 1-15.

In some embodiments, n is selected from 10-20, and m is selected from 2-10.

In some embodiments, n:m=(1-10):1. In some embodiments, n:m is (1-5):1. In some embodiments, n:m is (1.1-1.2):1, 2:1, 3:1, or 4:1.

In some embodiments, x₁ is selected form 1-30. In some embodiments, x₁ is 3, 4, 5, 6, 7, 8, 9, or 10.

In some embodiments, the product of (n₁+m₁) and x₁ is not less than 5. In some embodiments, the product of (n₁+m₁) and x₁ is not less than 9. In some embodiments, the product of (n₁+m₁) and x₁ is not less than 15. In some embodiments, the product of (n₁+m₁) and x₁ is within a range of 6-200. In some embodiments, the product of (n₁+m₁) and x₁ is within a range of 6-150. In some embodiments, the product of (n₁+m₁) and x₁ is within a range of 6-110. In some embodiments, the product of (n₁+m₁) and x₁ is within a range of 6-20, 5-40, 9-30, or 15-28. In some embodiments, the product of (n₁+m₁) and x₁ is within a range of 15-25. In some embodiments, the product of (n₁+m₁) and x₁ is 9, 10, 11, 12, 13, 14, 15, 16, 17, 18, 19, 20, 21, 22, 23, 24, 25, 26, 27, 28, 29, or 30.

In some embodiments, the product of n₁ and x₁ is within a range of 5-30, and the product of m₁ and x₁ is within a range of 0-15 or 1-15.

In some embodiments, the product of n₁ and x₁ is within a range of 10-20, and the product of m₁ and x₁ is within a range of 3-7.

In some embodiments, n₁:m₁ is (1-10):1. In some embodiments, n₁:m₁ is (2-4):1. In some embodiments, n₁:m₁ is 2:1, 3:1, or 4:1.

In some embodiments, n₁+m₁ is within a range of 2-10. In some embodiments, n₁+m₁ is 3, 4, 5, or 6.

In some embodiments, x₂ is selected from 1-30. In some embodiments, x₂ is 3, 4, 5, 6, 7, 8, 9, or 10.

In some embodiments, the product of (n₂+m₂) and x₂ plus n₃ and m₃ is not less than 5. In some embodiments, the product of (n₂+m₂) and x₂ plus n₃ and m₃ is not less than 9. In some embodiments, the product of (n₂+m₂) and x₂ plus n₃ and m₃ is not less than 12. In some embodiments, the product of (n₂+m₂) and x₂ plus n₃ and m₃ is not less than 15. In some embodiments, the product of (n₂+m₂) and x₂ plus n₃ and m₃ is within a range of 6-200. In some embodiments, the product of (n₂+m₂) and x₂ plus n₃ and m₃ is within a range of 6-150. In some embodiments, the product of (n₂+m₂) and x₂ plus n₃ and m₃ is within a range of 6-110. In some embodiments, the product of (n₂+m₂) and x₂ plus n₃ and m₃ is within a range of 6-20, 5-40, or 10-30. In some embodiments, the product of (n₂+m₂) and x₂ plus n₃ and m₃ is within a range of 13-21. In some embodiments, the product of (n₂+m₂) and x₂ plus n₃ and m₃ is 11, 12, 13, 14, 15, 16, 17, 18, 19, 20, 21, 22, 23, 24, or 25.

In some embodiments, (the product of n₂ and x₂ plus n₃):(the product of m₂ and x₂ plus m₃)=(1-10):1. In some embodiments, (the product of n₂ and x₂ plus n₃):(the product of m₂ and x₂ plus m₃) is (1-4):1. In some embodiments, (the product of n₂ and x₂ plus n₃):(the product of m₂ and x₂ plus m₃) is (1.1-1.2):1.

In some embodiments, m₃ is 0, n₃ is 1, the product of n₂ and x₂ is within a range of 6-12, and the product of m₂ and x₂ is within a range of 6-12.

In some embodiments, the product of n₂ and x₂ is 6, 8, or 10, and the product of m₂ and x₂ is 6, 8, or 10.

One or more embodiments of the present disclosure provide a membrane disruptive peptide nanoparticle formed by self-assembly of a membrane disruptive peptide in an aqueous medium.

One or more embodiments of the present disclosure provide a method for preparing a membrane disruptive peptide nanoparticle, including following operations: dissolving the membrane disruptive peptide in an organic solvent or a hydrochloric acid solution with a pH of 1.5-2.5 to obtain a solution, then dropwise adding the obtained solution into water under a stirring state, continuing stirring, and dialyzing to remove the solvent, thereby obtaining the membrane disruptive peptide nanoparticle.

In some embodiments, the organic solvent is N,N-dimethylformamide.

In some embodiments, a ratio of the membrane disruptive peptide, the organic solvent or the hydrochloric acid solution, and water is (20 mg to 40 mg):1 mL:(2 mL to 5 mL).

In some embodiments, the method for preparing the membrane disruptive peptide nanoparticles includes following operations: dissolving the membrane disruptive peptide in N,N-dimethylformamide according to a ratio of (10 mg to 30 mg):1 mL to obtain a solution, then adding the obtained solution dropwise to 2 mL to 5 mL of water under a stirring state at a rotation speed of 400 to 800 rpm, continuing to stir at a rotation speed of 200 to 600 rpm for 8 to 20 minutes, and dialyzing in water using a dialysis bag with a molecular weight cutoff of 10000 to 20000 Da to remove the solvent, thereby obtaining the membrane disruptive peptide nanoparticles.

One or more embodiments of the present disclosure provide a use of the membrane disruptive peptide or a stereoisomer thereof or a pharmaceutically acceptable salt thereof in preparation of a medicament for preventing and/or treating a tumor.

One or more embodiments of the present disclosure provide a use of the membrane disruptive peptide nanoparticles in preparation of a medicament for preventing and/or treating a tumor.

In some embodiments, the tumor includes a pancreatic cancer, a melanoma, a colorectal cancer, a colon cancer, a lung cancer, a tongue squamous cell carcinoma, a cervical cancer, an ovarian cancer, an osteosarcoma, a liver cancer, a breast cancer, a bladder cancer, or an epithelial ovarian cancer.

One or more embodiments of the present disclosure provide a medicament for preventing and/or treating a tumor, prepared from an active ingredient and a pharmaceutically acceptable excipient; and the active ingredient includes the membrane disruptive peptide or the stereoisomer thereof or the pharmaceutically acceptable salt thereof, and/or the membrane disruptive peptide nanoparticles.

One or more embodiments of the present disclosure provide a method for preventing and/or treating a tumor; and the method includes: administering a safe and effective amount of the membrane disruptive peptide or the stereoisomer thereof or the pharmaceutically acceptable salt thereof to a patient; and/or administering a safe and effective amount of the membrane disruptive peptide nanoparticles to a patient; and/or administering a safe and effective amount of the medicament to a patient.

Compounds of formula (I) to formula (III) of the present disclosure may be used in combination with other known anti-tumor medicaments. When administered in combination, the compounds of formula (I) to formula (III) and the known medicament may be separate administration units or form a combined administration unit. The compounds of formula (I) to formula (III) may be administered simultaneously with or separately from the other known anti-tumor medicaments. When the compounds of formula (I) to formula (III) are administered simultaneously with one or several other medicaments, a pharmaceutical composition containing the one or several known medicaments and the compounds of formula (I) to formula (III) is preferably used. Drug combination also includes administering the compounds of formula (I) to formula (III) and one or more other known medicaments during an overlapping time period. When the compounds of formula (I) to formula (III) are used in the drug combination with one or more other known medicaments, a dose of the compounds of formula (I) to formula (III) or the known medicament may be the same as a dose when used alone, or may be lower than the dose when used alone.

The medicament or the active ingredient that may be used in drug combination with the compounds of formula (I) to formula (III) include, but are not limited to: an immune checkpoint inhibitor, an estrogen receptor modulator, an androgen receptor modulator, a retinoid receptor modulator, a cytotoxin/cytostatic agent, an antiproliferative agent, a protein transferase inhibitor, an HMG-CoA reductase inhibitor, an HIV protease inhibitor, a reverse transcriptase inhibitor, an angiogenesis inhibitor, an inhibitor of cell proliferation and survival signaling, an agent interfering with cell cycle checkpoints and an apoptosis inducer, a cytotoxic drug, a tyrosine kinase inhibitor, an EGFR inhibitor, a VEGFR inhibitor, a serine/threonine kinase inhibitor, a Bcr-Abl inhibitor, a c-Kit inhibitor, a Met inhibitor, a Raf inhibitor, a MEK inhibitor, an MMP inhibitor, a topoisomerase inhibitor, a histone deacetylase inhibitor, a proteasome inhibitor, a CDK inhibitor, a Bcl-2 family protein inhibitor, an MDM2 family protein inhibitor, an IAP family protein inhibitor, a STAT family protein inhibitor, a PI3K inhibitor, an AKT inhibitor, an integrin blocker, an interferon-α, an interleukin-12, a COX-2 inhibitor, a p53, a p53 activator, a VEGF antibody, an EGF antibody, etc.

In some embodiments, the medicament or the active ingredient that may be used in combination with the compounds of the formula (I) to formula (III) include, but are not limited to: aldesleukin, alendronic acid, interferon, atreleuton, allopurinol, allopurinol sodium, palonosetron hydrochloride, altretamine, aminoglutethimide, amifostine, amrubicin, amsacrine, anastrozole, dolasetron, aranesp, arglabin, arsenic trioxide, exemestane, azacitidine, azathioprine, Bacillus Calmette-Guerin (BCG) or Tice BCG, betadine, betamethasone acetate, betamethasone sodium phosphate preparation, bexarotene, bleomycin sulfate, bromodeoxyuridine, bortezomib, busulfan, calcitonin, alemtuzumab injection, capecitabine, carboplatin, Casodex, cefesone, cimzia (certolizumab pegol), daunorubicin, chlorambucil, cisplatin, cladribine, clodronate, cyclophosphamide, cytarabine, dacarbazine, dactinomycin D, daunorubicin liposome, dexamethasone, dexamethasone phosphate, estradiol valerate, denileukin diftitox, dipivefrin, deslorelin, dlarizine, diethylstilbestrol, Diflucan (fluconazole), docetaxel, doxifluridine, doxorubicin, dronabinol, holmium-166-chitosan complex, Eligard (leuprolide acetate), rasburicase, epirubicin hydrochloride, aprepitant, epirubicin, epoetin alfa, erythropoietin, eptaplatin, levamisole tablets, estradiol preparation, 17-β-estradiol, estramustine phosphate sodium, ethinyl estradiol, amifostine, etidronic acid, Velban (vinblastine), etoposide, fadrozole, tamoxifen preparation, filgrastim, finasteride, flutamide, formestane, 1-β-D-arabinofuranosylcytosine 5′-stearoyl phosphate, fotemustine, fulvestrant, gamma globulin, gemcitabine, gemtuzumab ozogamicin, imatinib mesylate, carmustine wafers, goserelin, granisetron hydrochloride, histrelin, Hycamtin (topotecan), hydrocortisone, erythro-hydroxy-nonyladenine, hydroxyurea, ibritumomab tiuxetan, idarubicin, ifosfamide, interferon α, interferon-α2, interferon α-2A, interferon α-2B, interferon α-n₁, interferon α-n₃, interferon β, interferon γ-la, interleukin-2, Intron A (interferon α-2B), Iressa (gefitinib), irinotecan, Kytril (granisetron), lentinan sulfate, letrozole, leucovorin, leuprolide, leuprolide acetate, levamisole, levoleucovorin calcium, levothyroxine sodium, levothyroxine sodium preparation, lomustine, lonidamine, dronabinol, mechlorethamine, methylcobalamin, medroxyprogesterone acetate, megestrol acetate, melphalan, conjugated estrogens, 6-mercaptopurine, mesna, methotrexate, methyl aminolevulinate, miltefosine, minocycline, mitomycin C, mitotane, mitoxantrone, trilostane, doxorubicin citrate liposome, nedaplatin, pegfilgrastim, oprelvekin, Neupogen (filgrastim), nilutamide, tamoxifen, NSC-631570, recombinant human interleukin 1-0, octreotide, ondansetron hydrochloride, prednisolone oral solution, oxaliplatin, paclitaxel, prednisone sodium phosphate preparation, pegaspargase, Pegasys (peginterferon α-2a), pentostatin, picibanil, pilocarpine hydrochloride, pirarubicin, mithramycin, porfimer sodium, prednimustine, prednisolone, prednisone, Premarin (conjugated estrogens), procarbazine, recombinant human erythropoietin, raltitrexed, Rebif, rhenium-186 etidronate, Rituxan (rituximab), Redoxon-A (ascorbic acid), romurtide, pilocarpine hydrochloride tablets, octreotide, sargramostim, semustine, sizofiran, sobuzoxane, methylprednisolone sodium succinate, pamidronic acid, stem cell therapy, streptozocin, strontium-89 chloride, levothyroxine sodium, tamoxifen, tamsulosin, tasosartan, tastolactone, Taxotere (docetaxel), tiazofurin, temozolomide, teniposide, testosterone propionate, methyltestosterone, thioguanine, thiotepa, thyroid stimulating hormone, tiludronic acid, topotecan, toremifene, tositumomab, trastuzumab, treosulfan, tretinoin, methotrexate tablets, trimelamol, trimetrexate, triptorelin acetate, triptorelin pamoate, tegafur-uracil (UFT), uridine, valrubicin, vesnarinone, vinblastine, vincristine, vindesine, vinorelbine, verrucarin, dexrazoxane, zinostatin stimalamer, Zofran (ondansetron), paclitaxel protein-stabilized formulation, acolbifene, interferon gamma-lb, Affinitak (antisense oligonucleotide), aminopterin, arzoxifene, asoprisnil, atamestane, atrasentan, BAY 43-9006 (sorafenib), Avastin (bevacizumab), CCI-779 (temsirolimus), CDC-501, Celebrex (celecoxib), Erbitux (cetuximab), clinatox, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronate, interferon gamma, Intron-PEG, ixabepilone, keyhole limpet hemocyanin (KLH), L-651582, lanreotide, lasofoxifene, Libra, lonafarnib, miproxifene, minodronic acid, MS-209, liposomal MTP-PE, MX-6, nafarelin, nemorubicin, novastat, nolatrexed, oblimersen, onco-TCS, osidem, paclitaxel polyglutamate, pamidronate sodium, PN-401, QS-21, quazepam, R-154, raloxifene, ranpirnase, 13-cis-retinoic acid, satraplatin, seocalcitol, T-138067, Tarceva (erlotinib), docosahexaenoic acid-paclitaxel, thymosin al, tegafur, tipifarnib, tirapazamine, TLK-286, toremifene, trans-MID-lo7R, vasisoltamab, vapreotide, vatalanib, verteporfin, vinflunine, Z-100, and zoledronic acid, or any combination thereof.

The medicament for the preventing and/or treating the tumor of the present disclosure may be used for non-human mammals or humans.

The medicament or method for the preventing and/or treating the tumor provided by the present disclosure includes (administering) the safe and effective amount of the active ingredient (i.e., the tertiary amine-modified membrane disruptive peptide of the present disclosure or the nanoparticles thereof) and the pharmaceutically acceptable excipient. When administering the medicament, the safe and effective amount of the active ingredient is administered to the mammal (e.g., the human) in need of treatment; and a dose for administration is a pharmaceutically recognized effective administration dose. Of course, a specific dose should also consider factors such as a route of the administration and a health state of a patient, which are within a skill range of a skilled physician.

The term “safe and effective amount” refers to an amount of the active ingredient sufficient to produce a significant improvement in a disease condition without causing a severe adverse side effect. The term “pharmaceutically acceptable excipient” refers to one or more compatible solid or liquid fillers or gel substances that are suitable for human use and possess sufficient purity and sufficiently low toxicity. The term “compatible” herein refers to that components of the composition can be mixed with the active ingredient of the present disclosure (the tertiary amine-modified polypeptide membrane disruptive material as shown in formula (I)) and with each other without significantly reducing a pharmacological efficacy of the active ingredient.

The pharmaceutically acceptable excipients used in the medicament for the preventing and/or treating a tumor of the present disclosure include, but are not limited to, one or more of following materials: a solvent, an excipient, a filler, a bulking agent, a binder, a humectant, a disintegrant, a dissolution retardant, an absorption accelerator, an adsorbent, a diluent, a solubilizer, an emulsifier, a lubricant, a wetting agent, a suspending agent, a flavoring agent, and a perfume.

For example, the pharmaceutically acceptable excipients include: cellulose and derivatives thereof (e.g., sodium carboxymethyl cellulose, sodium ethyl cellulose, cellulose acetate, etc.), gelatin, talc, a solid lubricant (e.g., stearic acid and magnesium stearate), calcium sulfate, a vegetable oil (e.g., a soybean oil, a sesame oil, a peanut oil, an olive oil, etc.), a polyol (e.g., a propylene glycol, a glycerol, a mannitol, a sorbitol, etc.), an emulsifiers (e.g., Tween®), a wetting agent (e.g., sodium lauryl sulfate), a coloring agent, a flavoring agent, a stabilizer, an antioxidant, a preservative, pyrogen-free water, etc.

A manner of an administration of the active ingredient or a pharmaceutical composition of the present disclosure is not particularly limited, and representative manners of the administration include (but are not limited to): oral administration, rectal administration, parenteral administration (intravenous, intramuscular, or subcutaneous), etc.

Solid dosage forms for an oral administration include a capsule, a tablet, a pill, a powder, and a granule.

In these solid dosage forms, the active ingredient is mixed with at least one conventional inert excipient (or carrier), such as sodium citrate or dicalcium phosphate, or mixed with following components:
(a) a filler or a bulking agent, e.g., starch, lactose, sucrose, glucose, mannitol, and silicic acid;
(b) a binder, e.g., hydroxymethyl cellulose, alginate, gelatin, polyvinylpyrrolidone, sucrose, and gum arabic;
(c) a humectant, e.g., glycerol;
(d) a disintegrant, e.g., agar, calcium carbonate, potato starch or tapioca starch, alginic acid, a certain complex silicate, and sodium carbonate;
(e) a retardant agent, e.g., paraffin;
(f) an absorption accelerator, e.g., a quaternary ammonium compound;
(g) a wetting agent, e.g., cetyl alcohol and glyceryl monostearate;
(h) an adsorbent, e.g., kaolin; and
(i) a lubricant, e.g., talc, calcium stearate, magnesium stearate, solid polyethylene glycol, sodium lauryl sulfate, or a mixture thereof. In the capsule, the tablets, and the pills, dosage forms may also contain a buffering agent.

The aforementioned solid dosage form may also be prepared with coatings and shells, such as enteric coatings and other materials well known in the art. The solid dosage form may contain an opacifying agent, and release of the active ingredient in such compositions may be controlled in a delayed manner to occur in a specific portion of a gastrointestinal tract. Examples of embedding components that may be used are polymeric substances and waxy substances.

A liquid dosage form for the oral administration includes a pharmaceutically acceptable emulsion, solution, suspension, syrup, or tincture. In addition to the active ingredient, the liquid dosage form may contain inert diluents conventionally used in the art, such as water or other solvents, solubilizers, and emulsifiers, e.g., ethanol, isopropyl alcohol, ethyl carbonate, ethyl acetate, propylene glycol, 1,3-butanediol, dimethylformamide, and oil, particularly cottonseed oil, peanut oil, corn germ oil, olive oil, castor oil, and sesame oil, or a mixture thereof. In addition to these inert diluents, the composition may also include an adjuvant such as a wetting agent, an emulsifying agent, a suspending agent, sweetener, flavoring agent, and fragrance.

In addition to the active ingredient, the suspension may contain a suspending agent, e.g., ethoxylated isooctadecanol, polyoxyethylene sorbitol and dehydrated sorbitol ester, microcrystalline cellulose, methanolic aluminum, and agar, or a mixture thereof.

Compositions for parenteral injection may include a physiologically acceptable sterile aqueous or anhydrous solution, dispersion, suspension, or emulsion, and a sterile powder for reconstitution into a sterile injectable solution or dispersion. A suitable aqueous and non-aqueous carrier, diluent, solvent, or excipient include water, ethanol, polyol, and a suitable mixture thereof.

Some embodiments of the present disclosure provide a tertiary amine-modified membrane disruptive peptide material. This macromolecular material is hydrophobic and electrically neutral at a normal physiological pH, and polypeptide fragments are hydrophobic and have a weak interaction with the cell membrane, thereby having an advantage of low toxicity to a normal tissue during in vivo circulation. Under a slightly acidic pH condition of a tumor tissue, a tertiary amine portion of the macromolecular material becomes protonated, thereby enabling the polypeptide fragments to form an amphiphilic structure composed of a hydrophobic domain and a cationic domain. As a result, the tertiary amine-modified membrane disruptive peptide material exhibits a strong interaction with a cell membrane and a potent membrane-disruptive activity, thereby efficiently and selectively killing a tumor cell and a bacterium. The membrane disruptive peptide macromolecular material of the present disclosure can be used to prepare an anti-tumor medicament, and has advantages of a good anti-tumor effect, a high selectivity, and a low toxicity. Moreover, since the polymer polypeptide has advantages of degradability and non-toxic degradation products, the polymer polypeptide has a broader biomedical application.

The following provides specific examples.

In the following examples, a polypeptide containing a mPEG group is synthesized by reacting mPEGy-COOH with an N-terminal primary amine of a polypeptide. A tertiary amine is subsequently introduced through a primary amine on a lysine side chain, and a series of membrane disruptive peptide macromolecular materials are obtained. A reaction scheme and corresponding abbreviations are as follows:

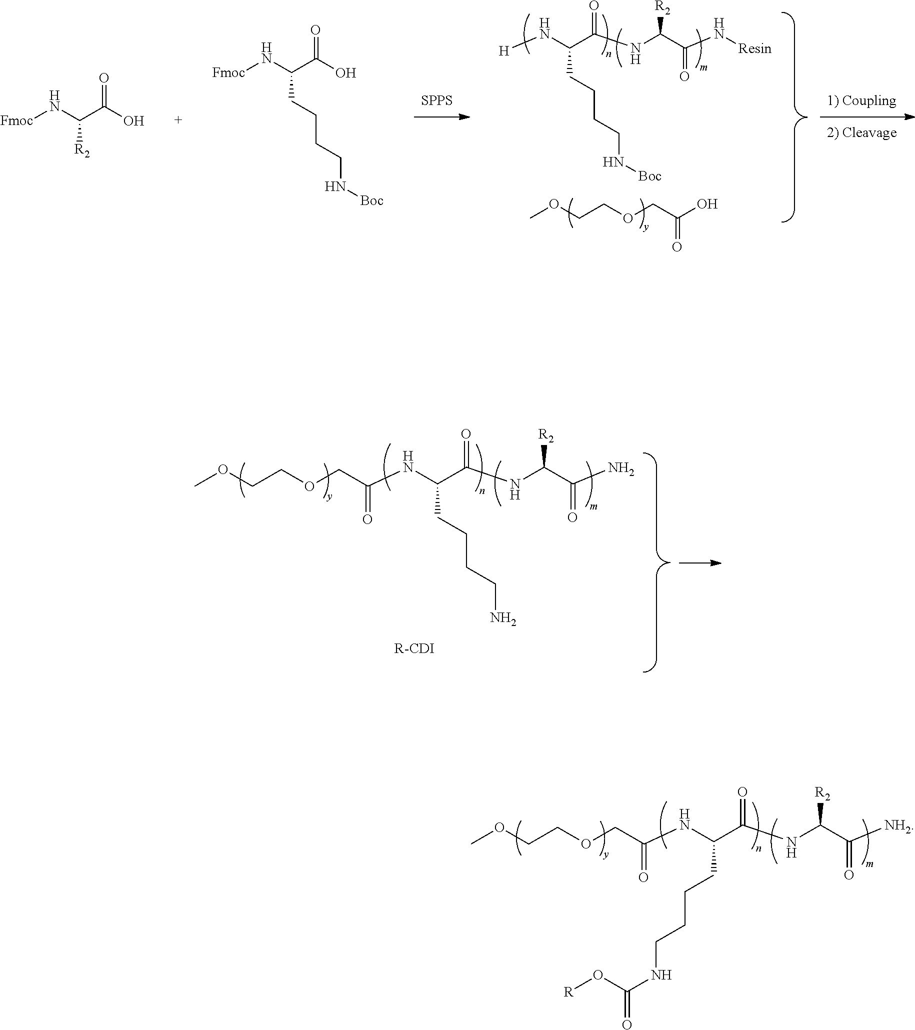

In a general reaction formula of the present disclosure, n lysines or tertiary amine-modified lysines, and m hydrophobic amino acids may be arranged in any determined sequence. For example, in the present disclosure and the following embodiments, the abbreviation (K)ₙ(W)ₘrefers to a polypeptide formed by n lysines (or tertiary amine-modified lysines) and m tryptophans arranged in a determined sequence via peptide bonds. The abbreviation (K)ₙ(F)ₘ refers to a polypeptide formed by n lysines (or tertiary amine-modified lysines) and m phenylalanines arranged in the determined sequence via the peptide bonds. Specific sequences are described in detail in the following embodiments.

A structural formula of the mPEGy-COOH is shown as follows:

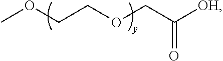

wherein y is 22, 45, or 113.

R-CDI is a product of a reaction between an alcohol having a tertiary amine structure (R—OH) and N,N′-carbonyldiimidazole (CDI), where R contains a tertiary amine structure that can be protonated with changes in pH.

The alcohol having the tertiary amine structure (R—OH) used for a side chain modification is the commercially available product. The R—OH first reacts with the CDI to prepare the R-CDI, which is then used for the side chain modification.

The R-CDI is prepared by reacting the R—OH with the CDI in dichloromethane. After the reaction is complete, deionized water is added to remove unreacted CDI. The product is separated by extraction with dichloromethane, dried with anhydrous magnesium sulfate, and a dichloromethane solution of R-CDI is obtained. The solvent is then removed under a reduced pressure.

A manner for the side chain modification is as follows: dissolving the polypeptide in N,N-dimethylformamide (DMF), adding the R-CDI (5-fold excess) and triethylamine to obtain a mixture, stirring the mixture for reaction for 12 h, removing the solvent under the reduced pressure, dissolving the residue in dimethyl sulfoxide (DMSO), dialyzing in the deionized water using a dialysis bag with a molecular weight cutoff of 3500 Da for 24 h and replacing the deionized water every 2 h, and lyophilizing the product to obtain the membrane disruptive peptide.

### Example 1: Sequence-Defined Polypeptides of 2-(dibutylamino)ethanol-Modified methoxy poly(ethylene glycol)-lysine and tryptophan

R—OH was 2-(dibutylamino)ethanol (DB), R₂ was a tryptophan residue, and y was 45. mPEG₄₅-COOH was reacted with an amino group in a sequence-defined polypeptide (K)ₙ(W)ₘ to obtain mPEG₄₅-(K)ₙ(W)ₘ. A tertiary amine was then modified onto a side chain of lysine via CDI to obtain a tertiary amine-modified sequence-defined polypeptide. Effects of different sequences and different molecular weights on pKa and pH-selective killing of the sequence-defined polypeptide were studied.

### (I) Preparation of 2-(dibutylamino)ethanol-Modified Sequence-Defined Polypeptides

A reaction scheme is as follows:

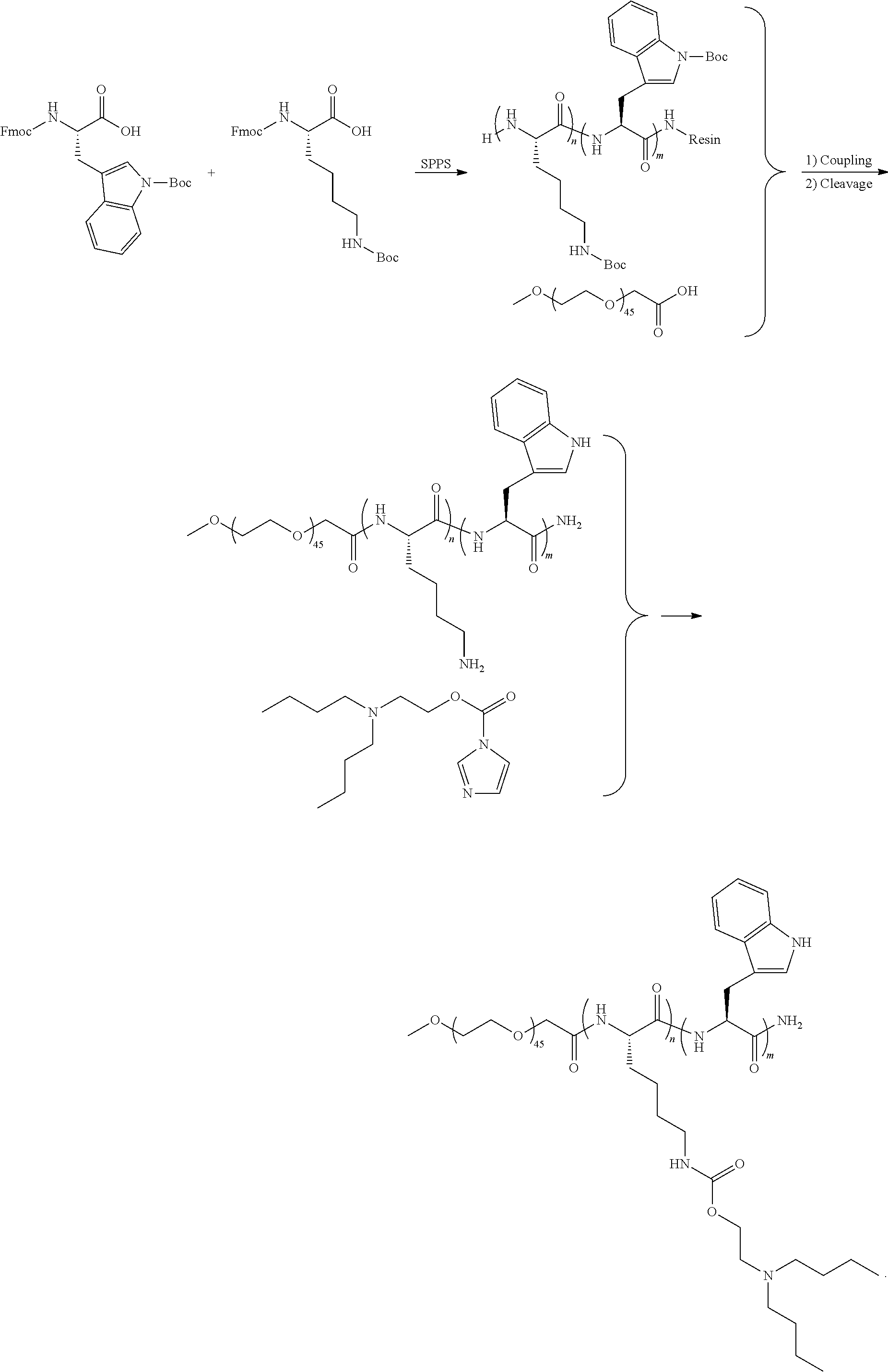

Specific steps were as follows:

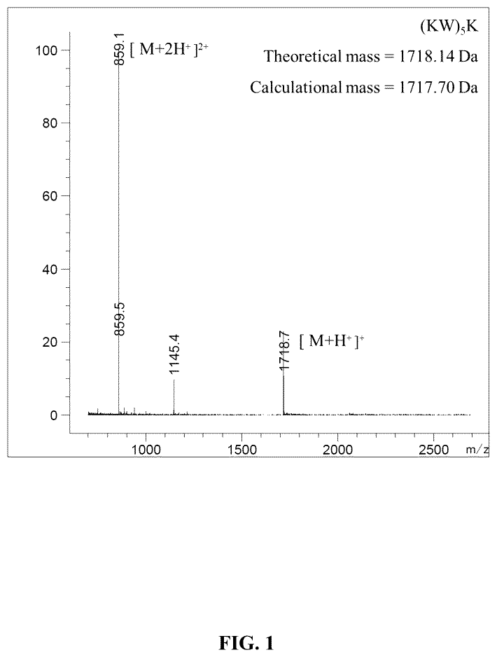

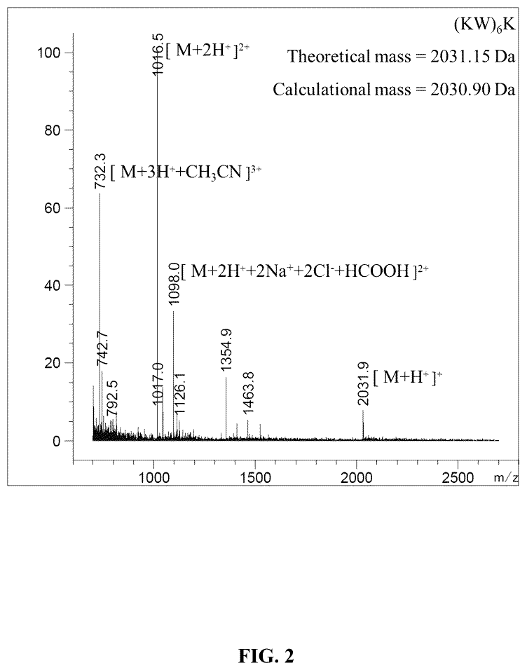

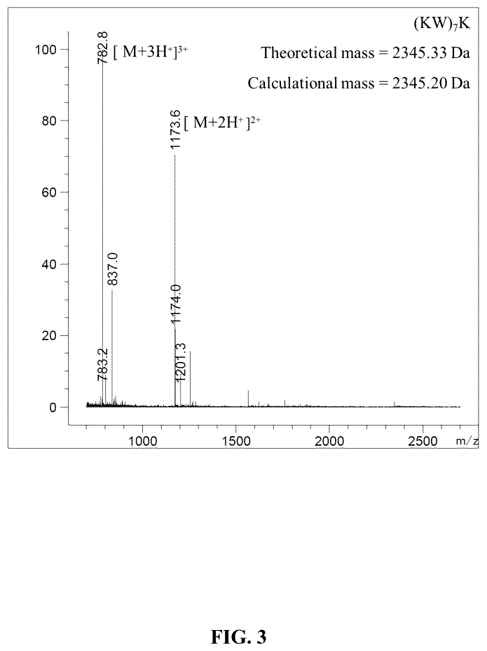

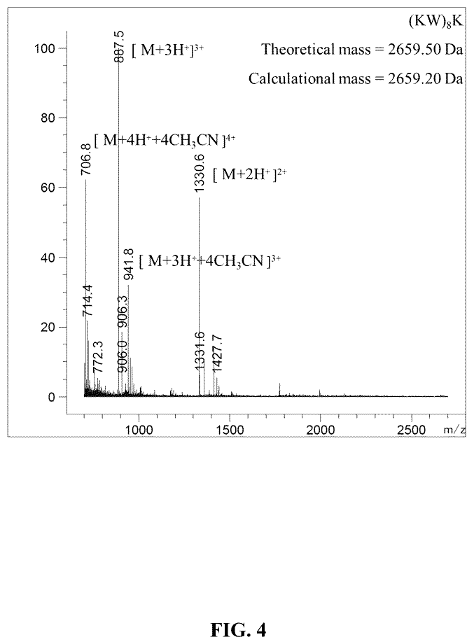

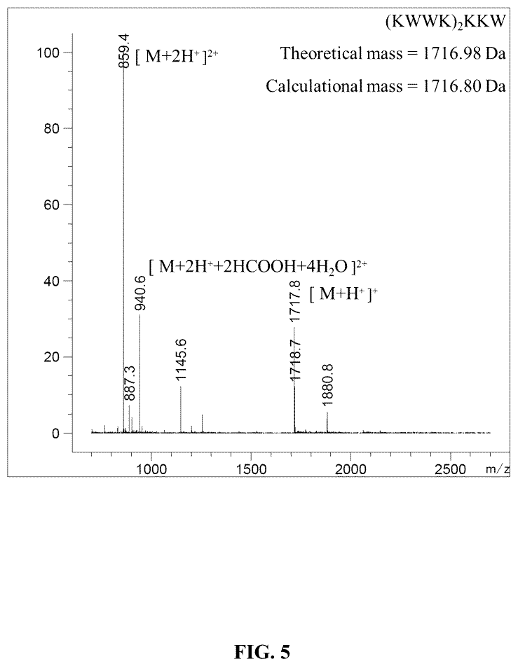

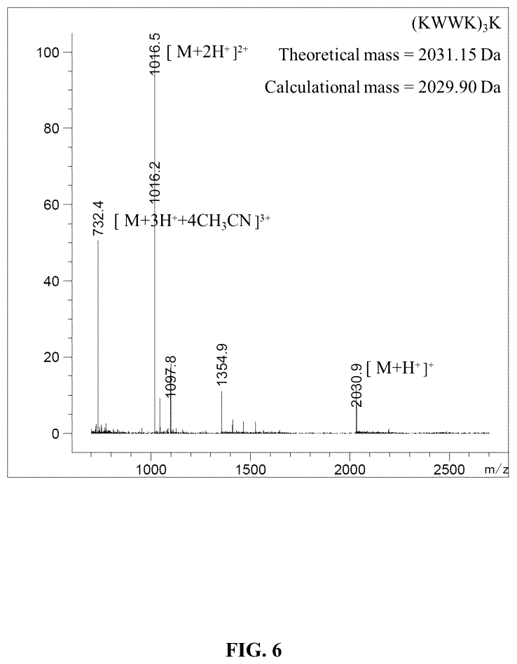

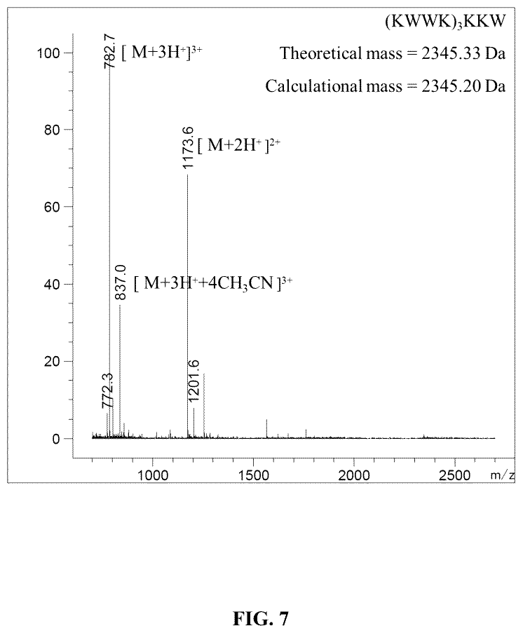

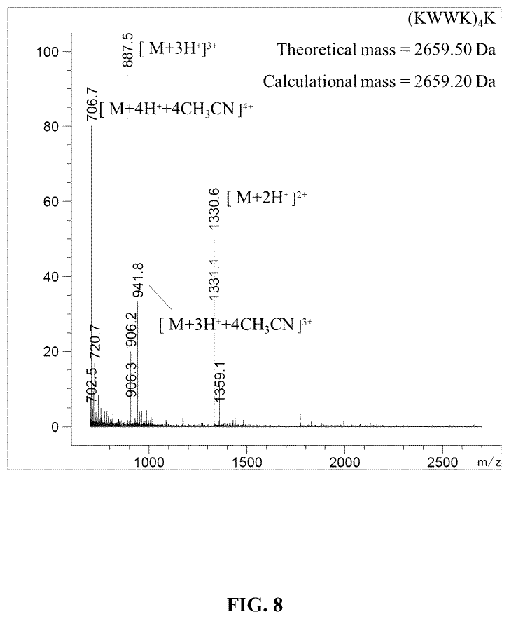

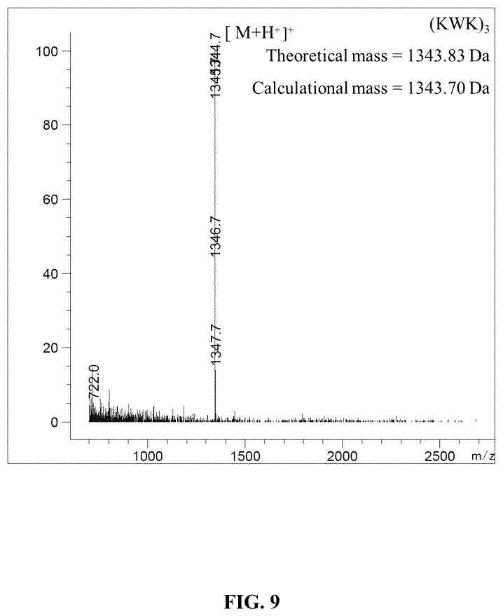

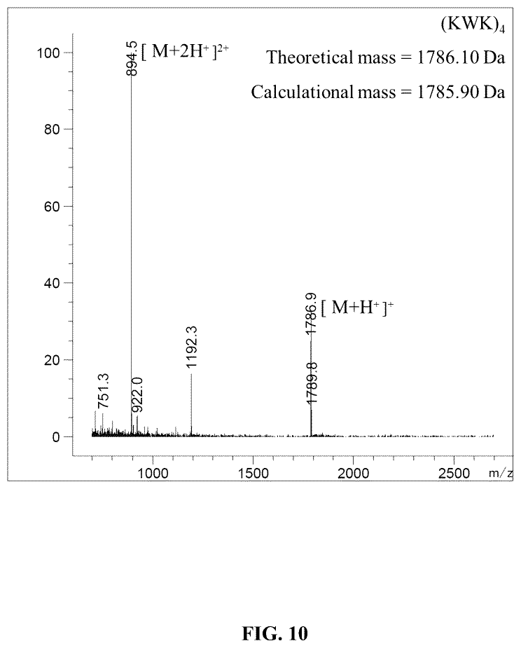

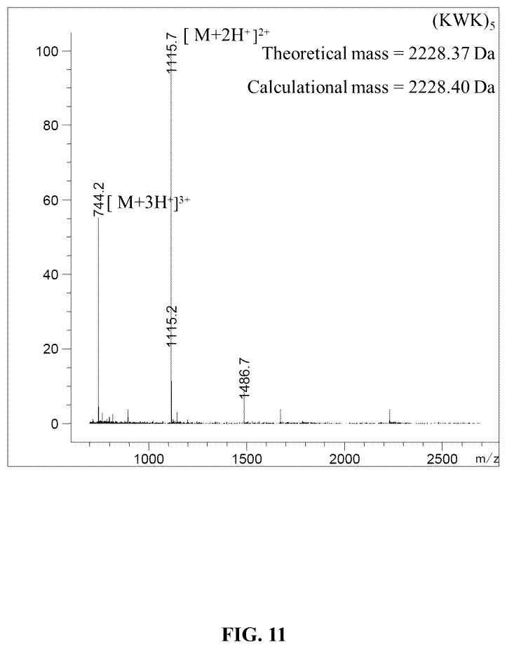

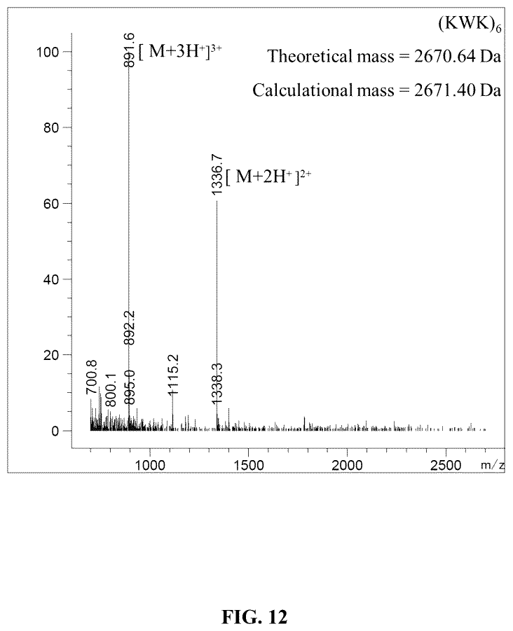

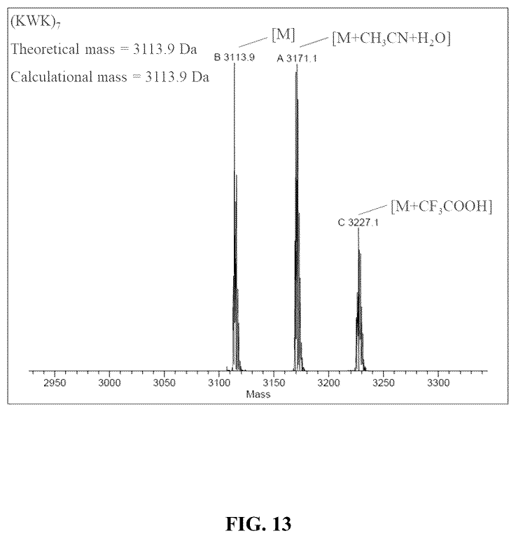

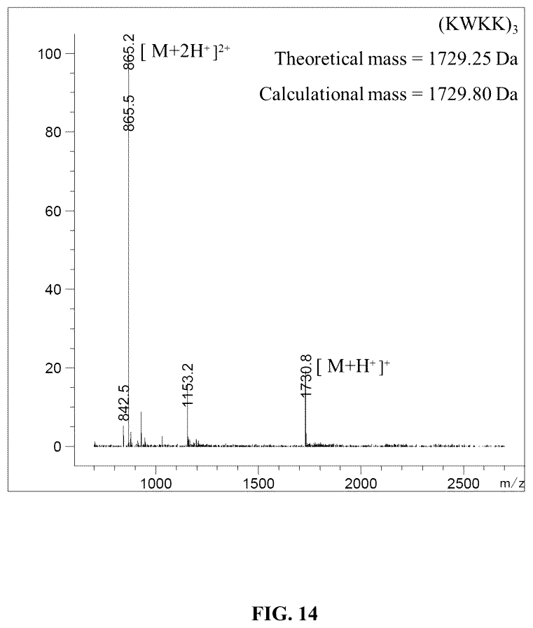

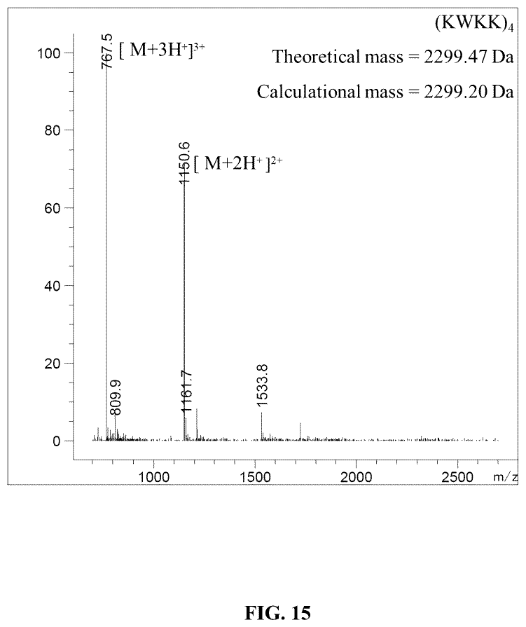

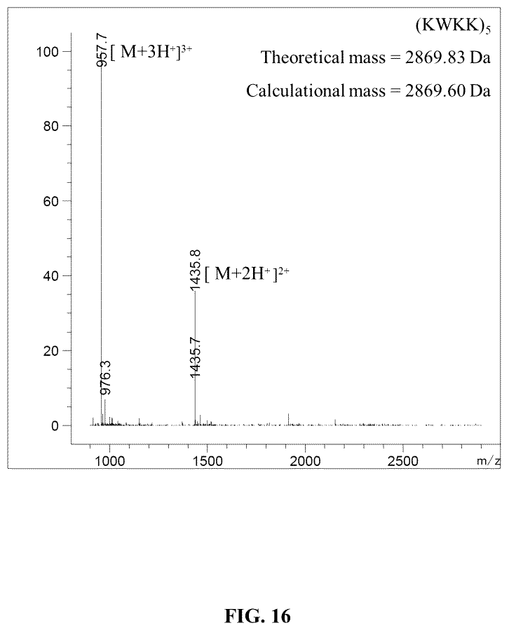

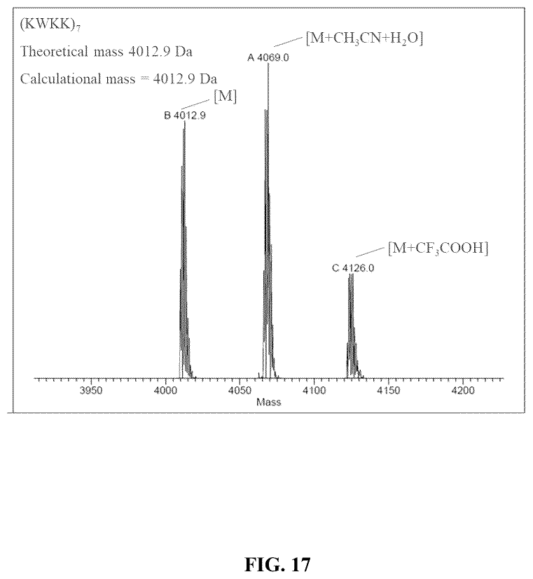

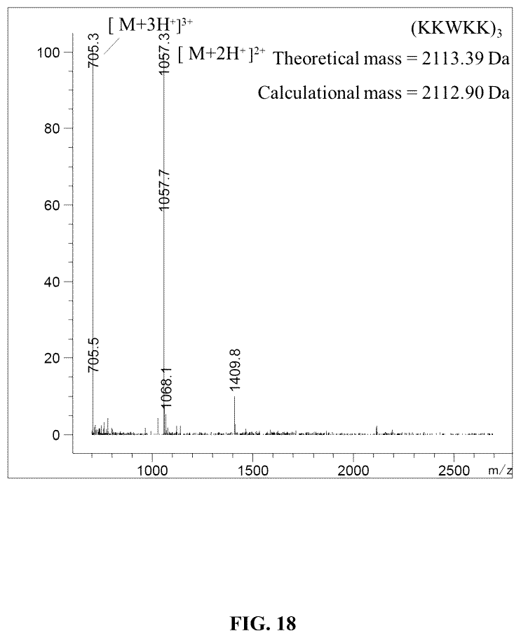

(1) Different sequences of H-[Lys(Boc)]4[Trp(Boc)]ₘ-Resin were synthesized by solid-phase peptide synthesis (SPPS). After each amino acid coupling, a completeness of an amino group reaction was tested using a ninhydrin reagent. A portion of the resin was cleaved with a cleavage solution composed of 95% trifluoroacetic acid (TFA), 2.5% H₂O, and 2.5% triisopropylsilane (TIS). The product was characterized by mass spectrometry to confirm a related structure, and the results are shown in FIG. 1 to FIG. 19, indicating that a series of polypeptide materials is successfully obtained.

(2) Different sequences of mPEG₄₅-[Lys(Boc)]4[Trp(Boc)]m-Resin were synthesized by the SPPS. After the reaction was complete, the completeness of the amino group reaction was tested using the ninhydrin reagent.

(3) The mPEG₄₅-[Lys(Boc)]i[Trp(Boc)]ₘ sequence-defined polypeptide was cleaved with a cleavage solution composed of 95% TFA, 2.5% H₂O, and 2.5% TIS for 3 h. The cleavage solution was concentrated with nitrogen gas and precipitated into ice-cold ether. A precipitate was obtained by centrifugation, then dissolved in deionized water, loaded into a 3500 Da dialysis bag, and dialyzed in deionized water for 24 h, and the deionized water was replaced every 2 h. After lyophilization, a white powder of mPEG₄₅-(K)ₙ(W)ₘ sequence-defined polypeptide was obtained. Specific sequences are as follows: mPEG₄₅-KWKWKWKWKWK, mPEG₄₅-KWKWKWKWKWKWK, mPEG₄₅-KWKWKWKWKWKWKWK, mPEG₄₅-KWKWKWKWKWKWKWKWK, mPEG₄₅-KWKWKWKWKWKWKWKWKWKWK, mPEG₄₅-KWWKKWWKKKW, mPEG₄₅-KWWKKWWKKWWKK, mPEG₄₅-KWWKKWWKKWWKKKW, mPEG₄₅-KWWKKWWKKWWKKWWKK, mPEG₄₅-KWWKKWWKKWWKKWWKKWWKK, mPEG₄₅-KWKKWKKWK, mPEG₄₅-KWKKWKKWKKWK, mPEG₄₅-KWKKWKKWKKWKKWK, mPEG₄₅-KWKKWKKWKKWKKWKKWK, mPEG₄₅-KWKKWKKWKKWKKWKKWKKWK, mPEG₄₅-KWKKKWKKKWKK, mPEG₄₅-KWKKKWKKKWKKKWKK, mPEG₄₅-KWKKKWKKKWKKKWKKKWKK, mPEG₄₅-KWKKKWKKKWKKKWKKKWKKKWKK, mPEG₄₅-KWKKKWKKKWKKKWKKKWKKKWKKKWKK, mPEG₄₅-KKWKKKKWKKKKWKK, mPEG₄₅-KKWKKKKWKKKKWKKKKWKK, mPEG₄₅-KKWKKKKWKKKKWKKKKWKKKKWKK, and mPEG₄₅-KKWKKKKWKKKKWKKKKWKKKKWKKKKWKK, which are respectively referred to as mPEG₄₅-(KW)₅K, mPEG₄₅-(KW)₆K, mPEG₄₅-(KW)₇K, mPEG₄₅-(KW)₈K, mPEG₄₅-(KW)₁₀K, mPEG₄₅-(KWWK)₂KKW, mPEG₄₅-(KWWK)₃K, mPEG₄₅-(KWWK)₃KKW, mPEG₄₅-(KWWK)₄K, mPEG₄₅-(KWWK)₅K, mPEG₄₅-(KWK)₃, mPEG₄₅-(KWK)₄, mPEG₄₅-(KWK)₅, mPEG₄₅-(KWK)₆, mPEG₄₅-(KWK)₇, mPEG₄₅-(KWKK)₃, mPEG₄₅-(KWKK)₄, mPEG₄₅-(KWKK)₅, mPEG₄₅-(KWKK)₆, mPEG₄₅-(KWKK)₇, mPEG₄₅-(KKWKK)₃, mPEG₄₅-(KKWKK)₄, mPEG₄₅-(KKWKK)₅, and mPEG₄₅-(KKWKK)₆.

(4) The CDI was placed in a round-bottom flask, and anhydrous dichloromethane was added to disperse the CDI with stirring (5 mL dichloromethane for 1.0 g CDI). The round-bottom flask was sealed with a rubber stopper, and 2-(dibutylamino)ethanol (3-fold excess relative to the CDI) was slowly added using a syringe. The solution gradually became clear. After reacting for 12 h, an equal volume of the deionized water relative to the dichloromethane was added. The stirring was continued until no bubbles emerged. A lower dichloromethane phase was collected using a separatory funnel. Anhydrous magnesium sulfate was added for drying for 2 h. The solid was removed by filtration. The solution was concentrated under a reduced pressure to obtain 2-(dibutylamino)ethanol-CDI (DB-CDI).

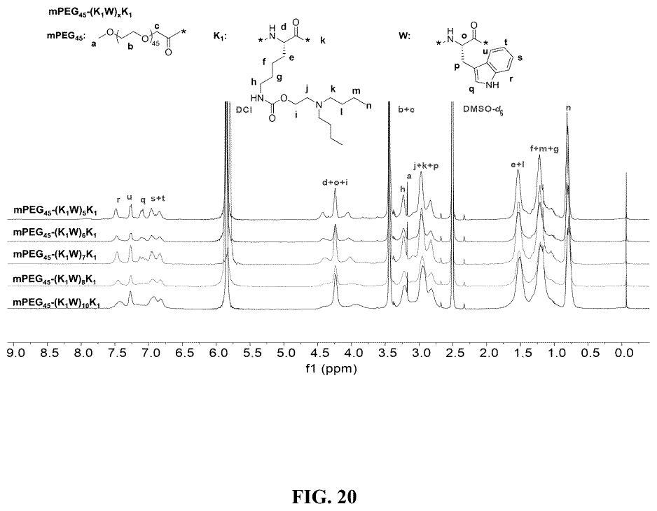

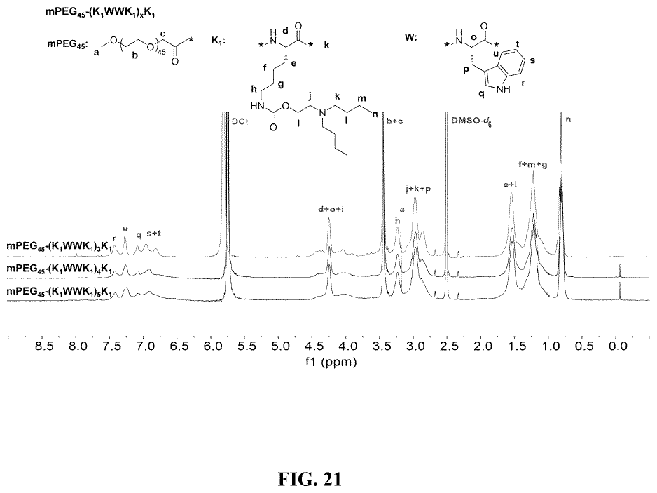

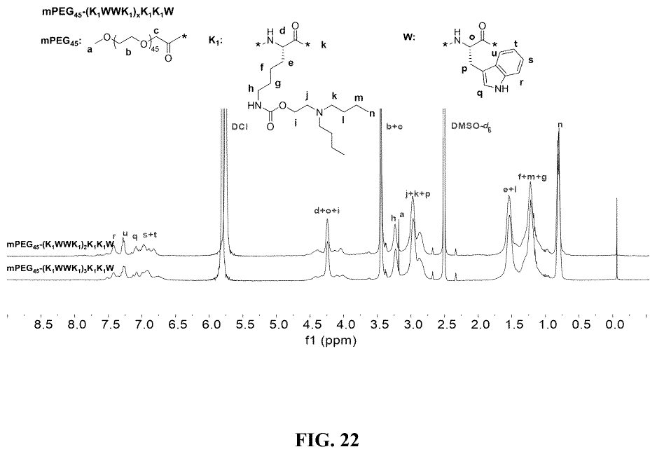

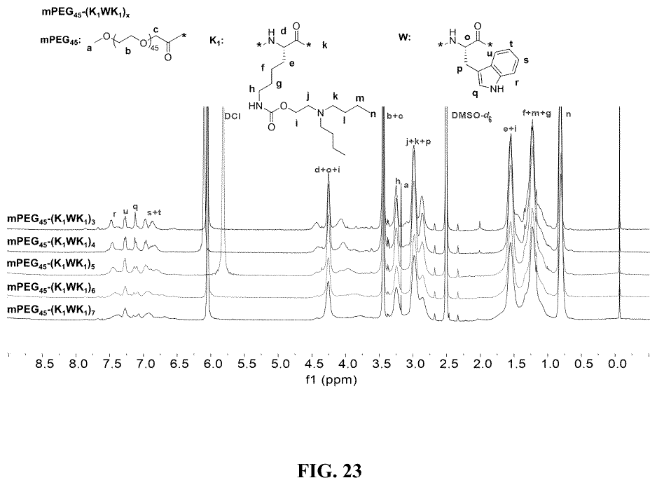

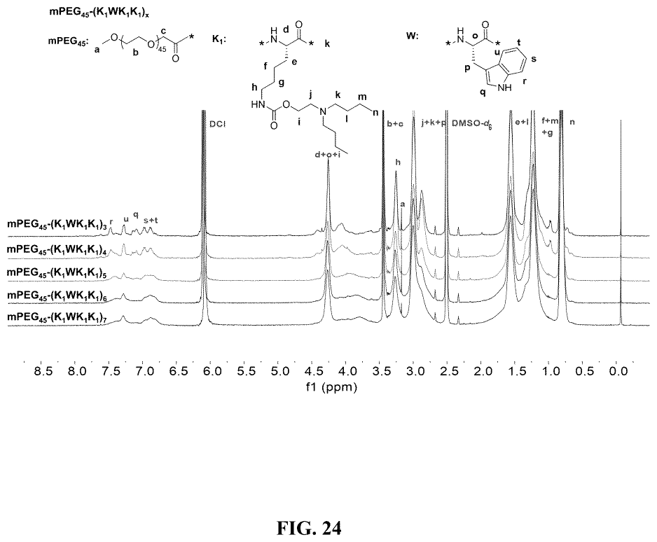

(5) The mPEG₄₅-(K)ₙ(W)ₘ sequence-defined polypeptide was dissolved in DMF and DB-CDI (5-fold excess) and triethylamine (an equimolar to lysine side chain amino groups) were added. The reaction proceeded for 12 h. The solvent was removed under the reduced pressure using an oil pump and the crude product was dissolved in DMSO. The solution was loaded into a 3.5 kDa dialysis bag and dialyzed in the deionized water for 24 h, and the deionized water was replaced every 2 h. After lyophilization, a DB-modified sequence-defined polypeptide was obtained. A DB-modified lysine is denoted as K₁. The obtained DB-modified sequence-defined polypeptides are referred to as: mPEG₄₅-(K₁W)₅K₁, mPEG₄₅-(K₁W)₆K₁, mPEG₄₅-(K₁W)₇K₁, mPEG₄₅-(K₁W)₈K₁, mPEG₄₅-(K₁W)₁₀K₁, mPEG₄₅-(K₁WWK₁)₂K₁K₁W, mPEG₄₅-(K₁WWK₁)₃K₁, mPEG₄₅-(K₁WWK₁)₃K₁K₁W, mPEG₄₅-(K₁WWK₁)₄K₁, mPEG₄₅-(K₁WWK₁)₅K₁, mPEG₄₅-(K₁WK₁)₃, mPEG₄₅-(K₁WK₁)₄, mPEG₄₅-(K₁WK₁)₅, mPEG₄₅-(K₁WK₁)₆, mPEG₄₅-(K₁WK₁)₇, mPEG₄₅-(K₁WK₁K₁)₃, mPEG₄₅-(K₁WK₁K₁)₄, mPEG₄₅-(K₁WK₁K₁)₅, mPEG₄₅-(K₁WK₁K₁)₆, mPEG₄₅-(K₁WK₁K₁)₇, mPEG₄₅-(K₁K₁WK₁K₁)₆, mPEG₄₅-(K₁K₁WK₁K₁)₄, mPEG₄₅-(K₁K₁WK₁K₁)₅, and mPEG₄₅-(K₁K₁WK₁K₁)₆. Structures of the DB-modified sequence-defined polypeptides were characterized by proton nuclear magnetic resonance (¹H NMR) spectra (as shown in FIG. 20 to FIG. 25). It can be seen from FIG. 20 to FIG. 25 that a peak corresponding to a methylene group of a lysine side chain linked to a primary amine group disappears, indicating that a coupling reaction is complete.

Structural formulas of the DB-modified sequence-defined polypeptides prepared in this example are as follows:

| | |
|---|---|
| mPEG₄₅-(K₁W)₅K₁ | mPEG₄₅-(K₁W)₆K₁ |
| 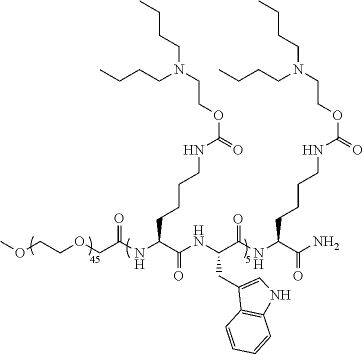 | 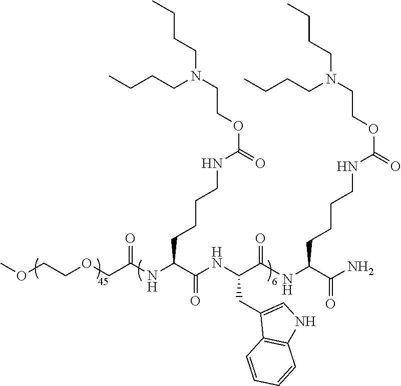 |
| mPEG₄₅-(K₁W)₇K₁ | mPEG₄₅-(K₁W)₈K₁ |
| 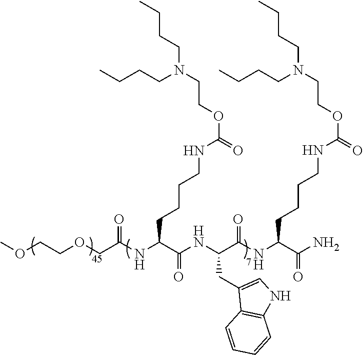 | 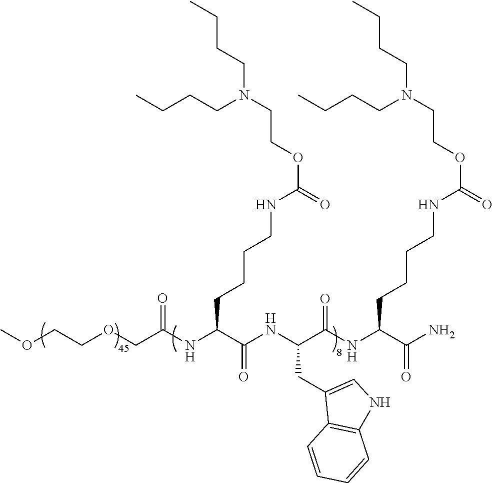 |
| mPEG₄₅-(K₁W)₁₀K₁ | mPEG₄₅-(K₁WWK₁)₂K₁K₁W |
| 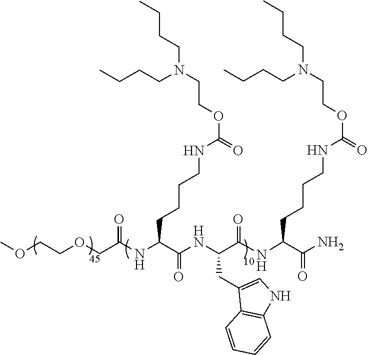 | 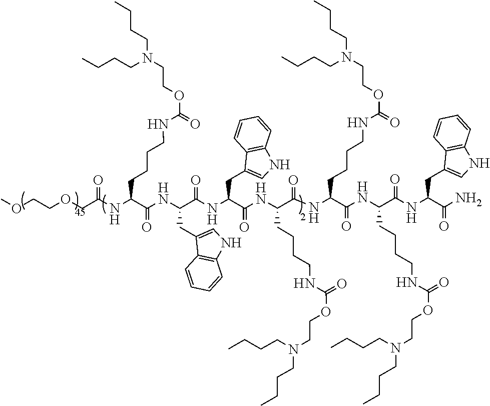 |
| mPEG₄₅-(K₁WWK₁)₃K₁ | mPEG₄₅-(K₁WWK₁)₃K₁K₁W |
| 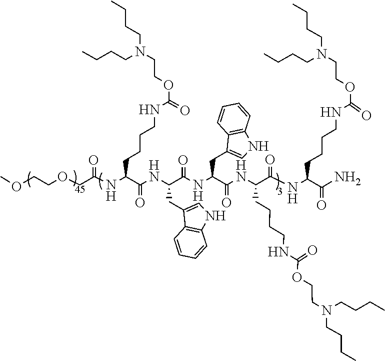 | 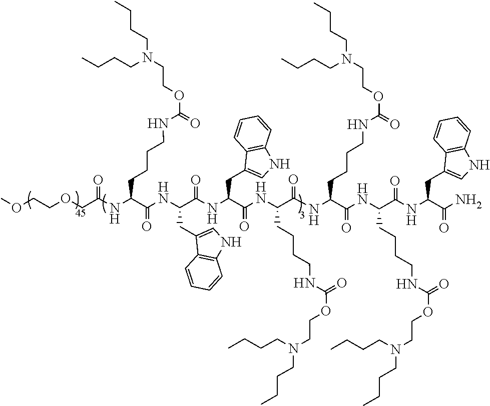 |
| mPEG₄₅-(K₁WWK₁)₄K₁ | mPEG₄₅-(K₁WWK₁)₅K₁ |
| 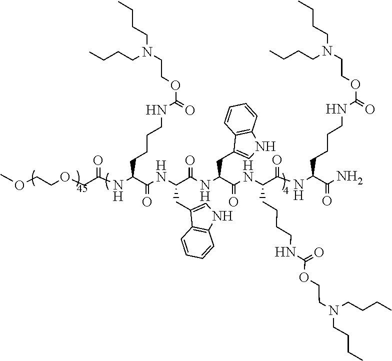 | 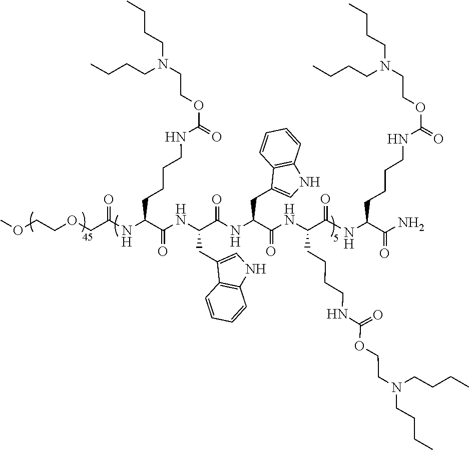 |
| mPEG₄₅-(K₁WK₁)₃ | mPEG₄₅-(K₁WK₁)₄ |
| 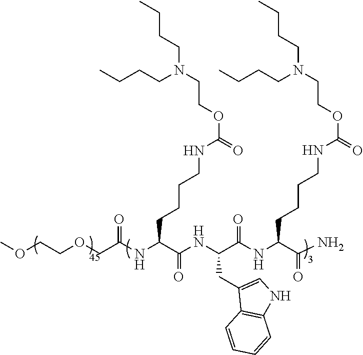 | 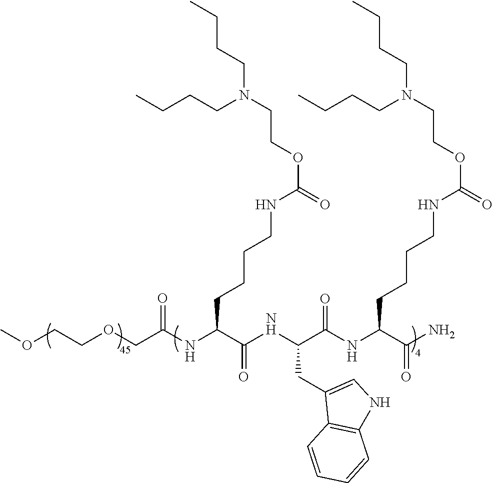 |
| mPEG₄₅-(K₁WK₁)₅ | mPEG₄₅-(K₁WK₁)₆ |
| 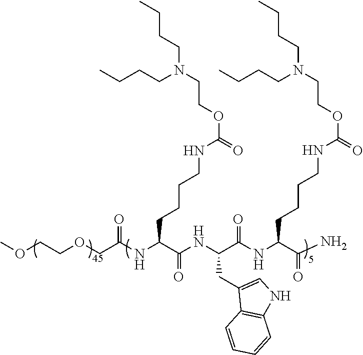 | 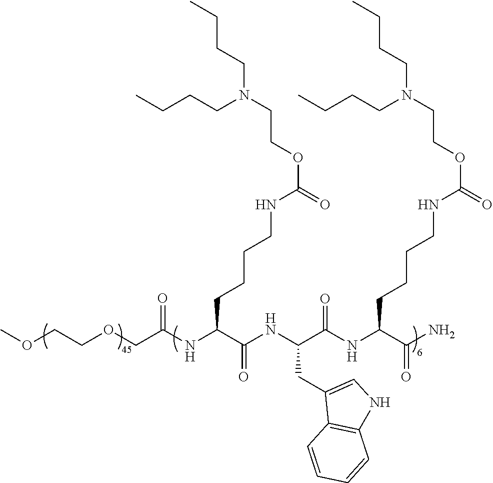 |
| mPEG₄₅-(K₁WK₁)₇ | MPEG₄₅-(K₁WK₁K₁)₃ |
| 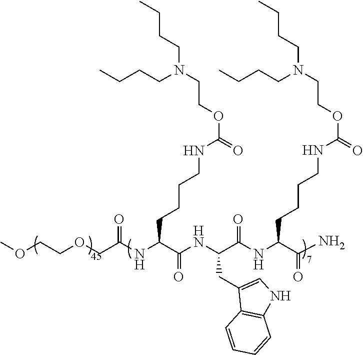 | 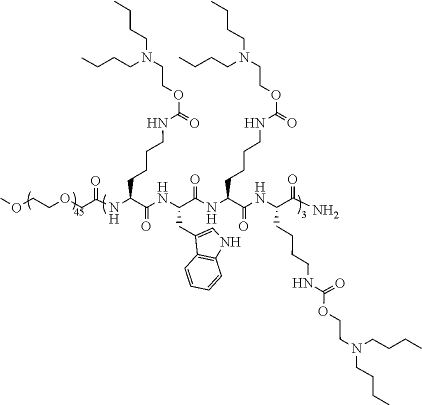 |
| mPEG₄₅-(K₁WK₁K₁)₄ | mPEG₄₅-(K₁WK₁K₁)₅ |
| 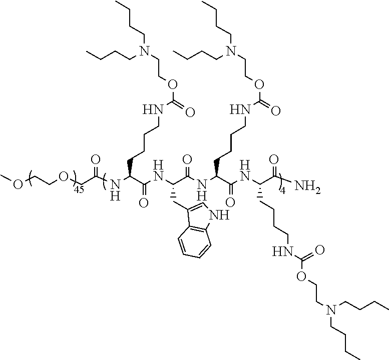 | 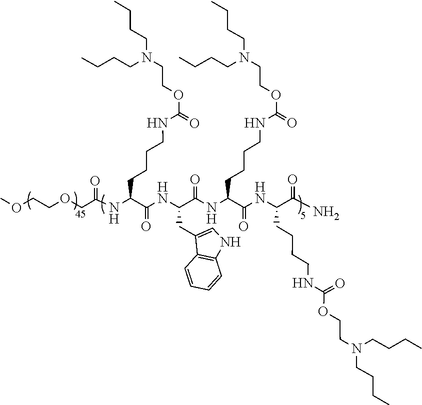 |
| mPEG₄₅-(K₁WK₁K₁)₆ | mPEG₄₅-(K₁WK₁K₁)₇ |
| 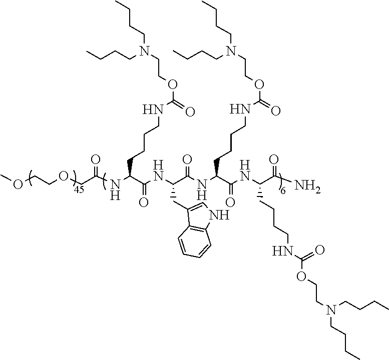 | 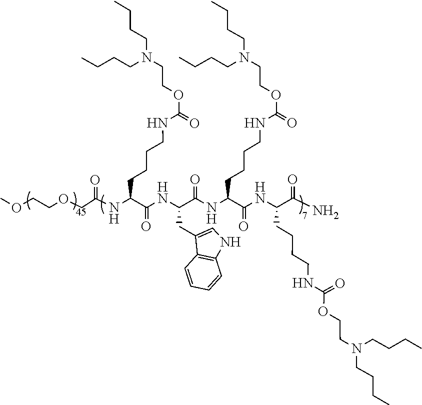 |
| mPEG₄₅-(K₁K₁WK₁K₁)₃ | mPEG₄₅(K₁K₁WK₁K₁)₄ |
| 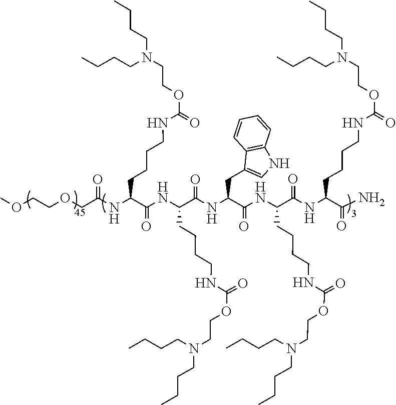 | 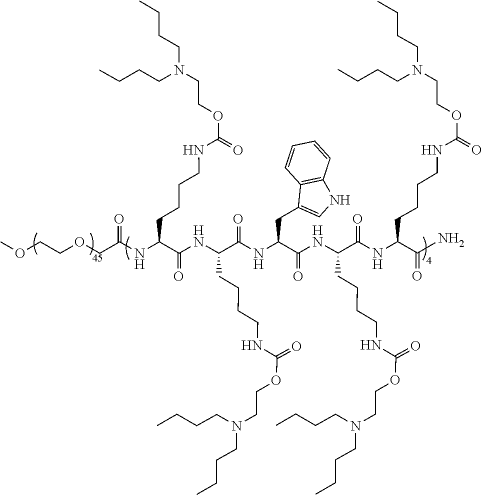 |
| mPEG₄₅-(K₁K₁WK₁K₁)₅ | mPEG₄₅-(K₁K₁WK₁K₁)₆ |
| 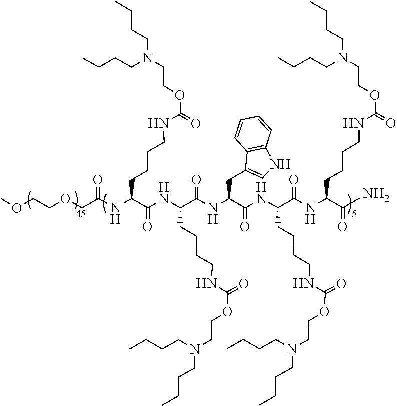 | 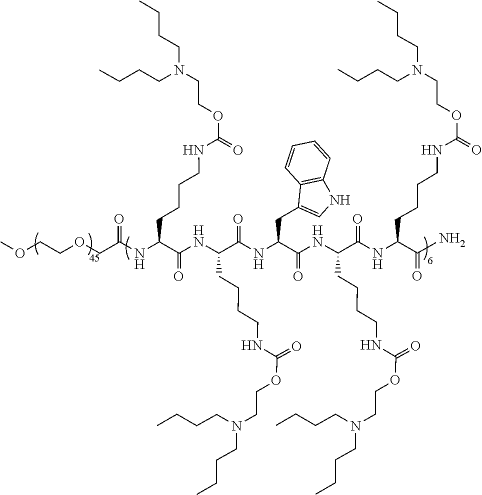 |

### (II) Determination of pKa Value of Polypeptide

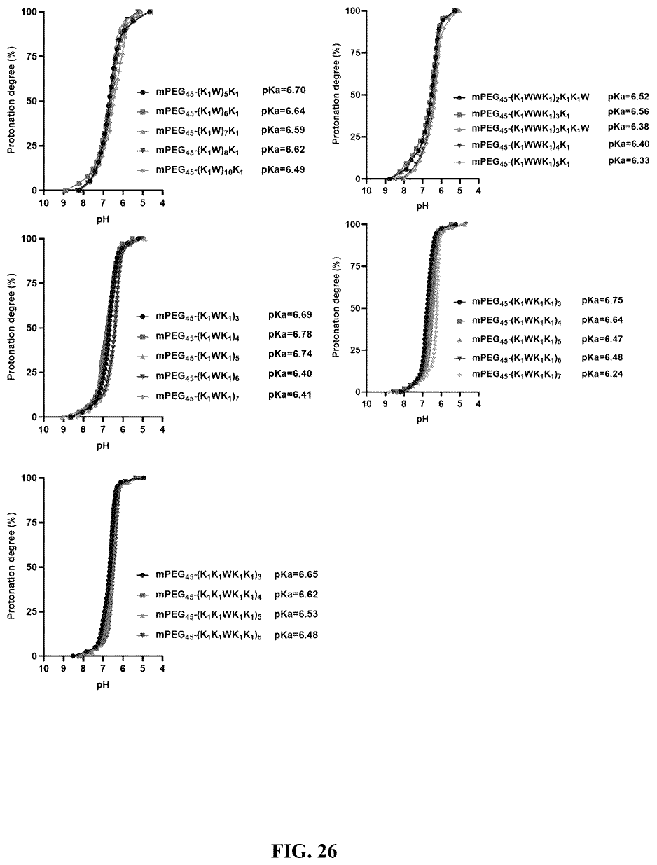

100 μL of concentrated hydrochloric acid (37%, 12 mol) was added to 100 mL of the deionized water and fully mixed to obtain a clear and transparent solution. 10 mg of the sequence-defined polypeptide material prepared in step (5) was dissolved in 10 mL of the hydrochloric acid solution. A pH probe was immersed below a liquid surface. Under a stirring condition (a stirring speed Mot of 3), a titration was performed using a 0.5 M sodium hydroxide titrant. After a pH meter stabilized, a reading was recorded. The titration continued until the reading reached pH=11. A derivative of a titration curve was taken to obtain extremum points, which were defined as points of protonation degrees of 1 and 0, respectively, thereby establishing a pH-protonation degree curve. According to a definition of a dissociation equilibrium constant (pKa), a pH corresponding to a protonation degree of 0.5 for the polypeptide was defined as a pKa of the material. The results are shown in FIG. 26. The protonation degree of these polypeptides exhibits a sharp transition with changing pH. As the molecular weight increases, the pKa of the polypeptides shows a decreasing trend.

### (III) Cytotoxicity of Sequence-Defined Polypeptides at Different pH Values

10 mg of the polypeptide prepared in step (5) was placed in a 5 mL sample vial. 0.3 mL of DMF was added for fully dissolution for later use. Separately, 0.9 mL of sterile water was added into a pre-sterilized 25 mL round-bottom flask on a magnetic stirrer. The solution was stirred at a speed of RPM=600 r/min. A DMF solution of the polypeptide was added dropwise to the round-bottom flask using a pipette. Stirring was continued at RPM=400 r/min for 15 min. Dialysis was then performed using a dialysis bag with a molecular weight cut-off of 14000 Da in 4 μL of ultrapure water at 4° C. for 24 h. The ultrapure water was replaced every 1 h during first 6 h and every 6 h for subsequent 18 h, yielding polypeptide nanoparticles. After the dialysis, the solution was taken out using the pipette, quantified, and stored in a 4° C. refrigerator.

A killing effect of the polypeptide nanoparticles on tumor cells under pH 7.4 and pH 6.8 conditions was evaluated by a Cell Counting Kit-8 (CCK-8) assay. A Dulbecco's Modified Eagle Medium (DMEM) was adjusted to pH=6.8 with a 6 mol/L hydrochloric acid solution and reserved for use. Mouse colorectal cancer cells MC38 (purchased from American Type Culture Collection (ATCC)) were cultured in the DMEM containing 10% (v/v) fetal bovine serum. Under pH 7.4 and pH 6.8 conditions, cells (at a concentration of 1×10⁵ cells/mL) were incubated with different concentrations of the polypeptide nanoparticles prepared in this example. After incubation for 24 h in a 37° C. incubator, the original medium was discarded. 100 μL of a CCK-8 solution was added, and incubation continued in a 37° C., CO₂ incubator for 2 h. An absorbance at 450 nm was measured using a microplate reader.

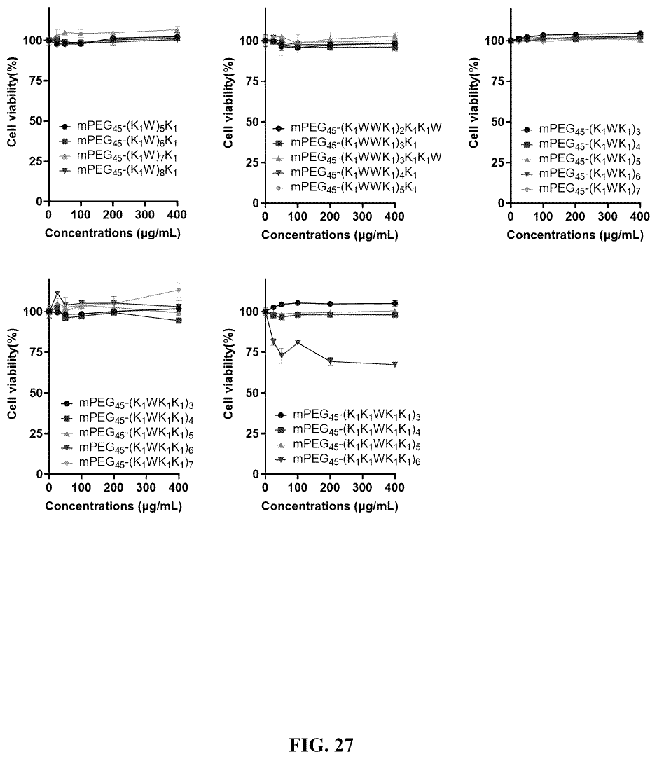

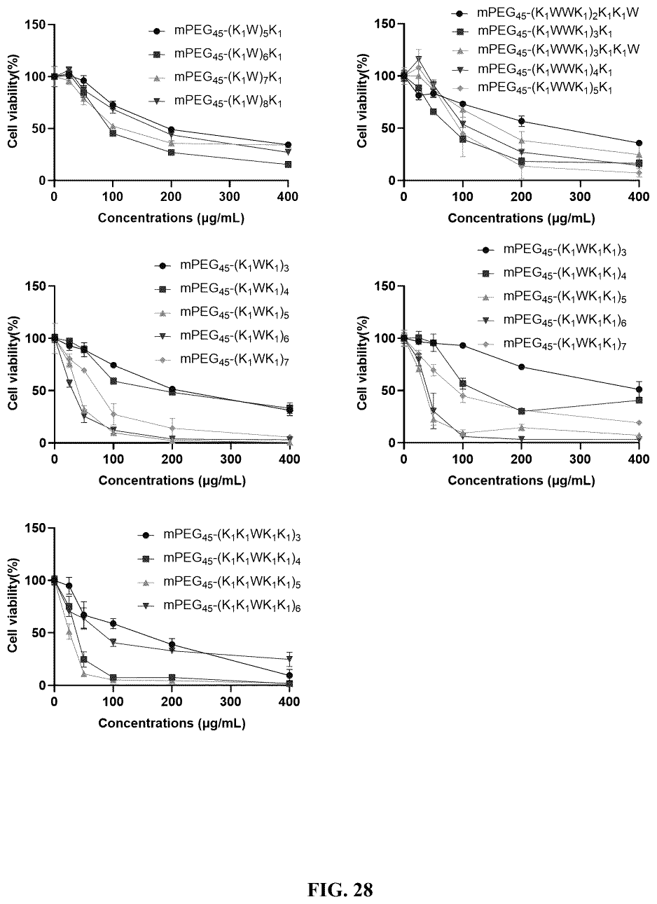

The test results are shown in FIG. 27 and FIG. 28. The sequence-defined polypeptides of DB-modified tryptophan and lysine are basically non-toxic to the tumor cells at pH 7.4, except that mPEG₄₅-(K₁K₁WK₁K₁)₆ exhibits certain toxicity. In contrast, the sequence-defined polypeptides of DB-modified tryptophan and lysine exhibit strong cytotoxicity at pH 6.8. This series of polypeptide materials exhibits an excellent pH-selective killing ability.

### (IV) In Vivo Toxicity of Sequence-Defined Polypeptides

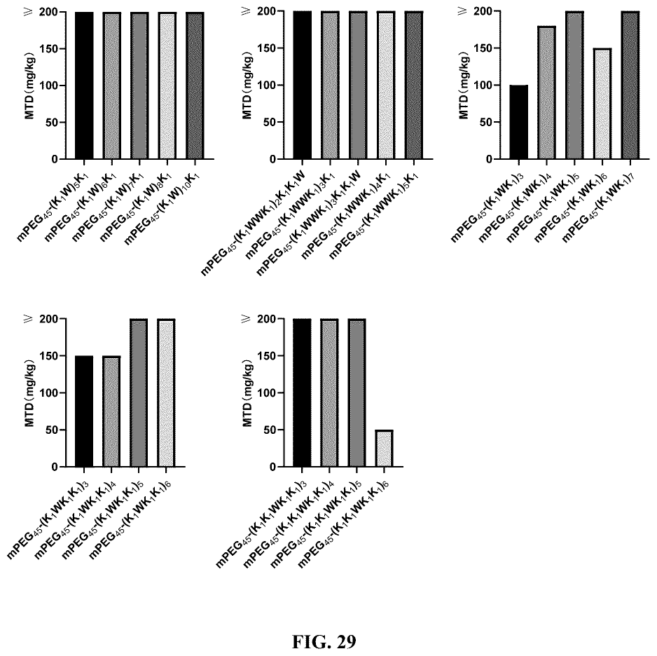

The toxicity of the polypeptides was tested in mice by determining the maximum tolerated dose (MTD). Female ICR mice aged six weeks (purchased from Hunan Shilaike Jingda Experimental Animal Co., Ltd.) were divided into ten major groups with similar average body weights, with five mice per group. Each polypeptide material was administered starting at a dose of 200 mg/kg body weight, with one female ICR mouse receiving a same dose. If no experimental animals died within 24 h, administration continued. If a death occurred, the dose was reduced by 10 mg/kg and administered again. This process continued until no mice died at a given dose. A maximum dose at which no mice died was recorded as a maximum tolerated dose for ICR mice, and the results were plotted in FIG. 29. As shown in FIG. 29, the sequence-defined polypeptide materials of this example all exhibit low in vivo toxicity, with most of the polypeptides showing MTD≥200 mg/kg.

(V) pH-Sensitive Membrane Disruptive Effect of Sequence-Defined Polypeptides by High-Content Observation

Panc02 cells expressing green fluorescent protein (GFP) on cell membrane and mCherry in cytoplasm were seeded into a 96-well plate at a density of (1.5-1.8)×10⁵ cells/mL and cultured overnight in an constant-temperature incubator for later use.

DMEM with different pH values were prepared. These media were used to prepare drug solutions with a concentration of 400 g/mL at the different pH values.

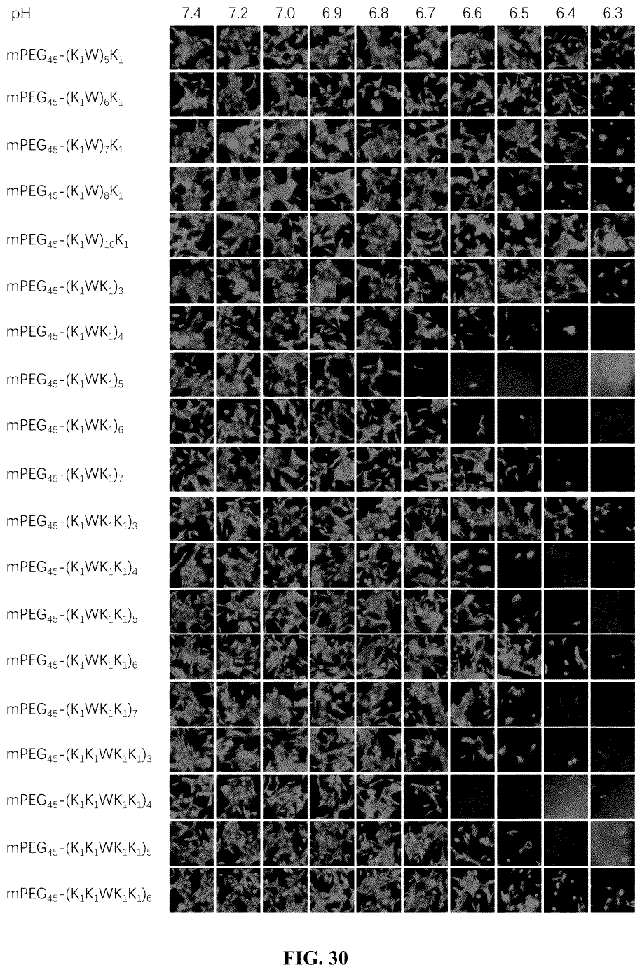

The prepared drug solutions described above were used to replace the original culture medium. After incubation for 4 h, imaging was performed. The results are shown in FIG. 30. These polypeptide materials exhibit pH-hypersensitive membrane disruptive effect, resulting in complete leakage of intracellular mCherry fluorescence within a narrow pH range.

This example indicates that the DB-modified lysine and tryptophan polypeptides possess excellent pH-selective cytotoxicity, low in vivo toxicity, and pH-hypersensitive membrane disruptive effect, enabling the complete leakage of intracellular mCherry fluorescence within a narrow pH range.

### (VI) Therapeutic Experiment of Sequence-Defined Polypeptides on MC38 Subcutaneous Colorectal Cancer Model

1) Establishment of MC38 subcutaneous colorectal cancer model in mice: MC38 cells were resuspended in serum-free medium and adjusted to a concentration of 1.0×10⁷ cells/mL. After removing hairs from right backs of female C₅₇BL/6 mice, 100 μL of cell suspension was subcutaneously injected at a depilated site.

2) When a tumor volume reached approximately 50 mm³, tumor-bearing mice were randomly divided into two groups, with three mice per group. The mice were subjected to tail vein injections according to following groups: PBS group and mPEG₄₅-(K₁WK₁)₅ (60 mg/kg) group. Administration was performed every other day for a total of six treatments.

3) Major and minor diameters of the tumors were measured and recorded to calculate the tumor volume. A body weight of the mice was measured and recorded.

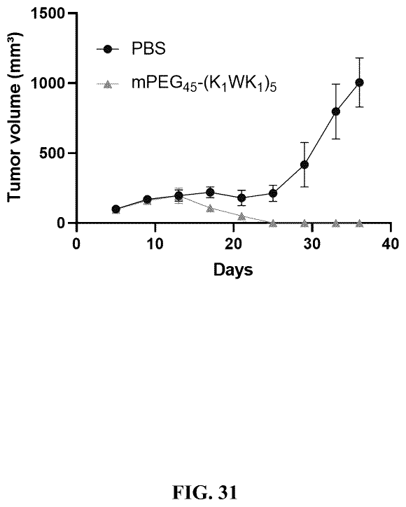

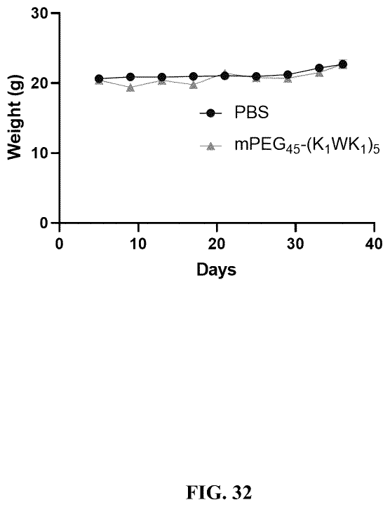

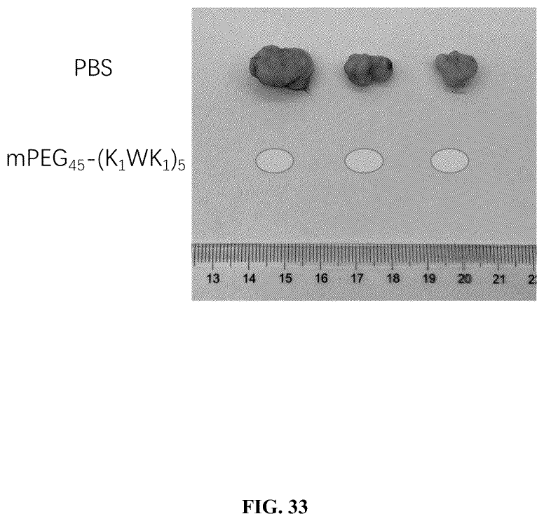

The results are shown in FIG. 31 to FIG. 33. mPEG₄₅-(K₁WK₁)₅ exhibits excellent anti-tumor effect, with complete tumor regression observed in all three mice. Furthermore, no reduction in the body weight is noted during a treatment period.

(VII) Membrane Disruptive Killing Mechanism of mPEG₄₅-(K₁WK₁)₅

1) Killing of mPEG₄₅-(K₁WK₁)₅ on the Panc02 cells expressing GFP on the cell membrane and mCherry in the cytoplasm: the cells were seeded into a confocal dish at a density of 100,000 cells per well and cultured overnight in a 37° C., CO₂ incubator. After adjusting parameters on the instrument, a drug solution containing 400 g/mL of mPEG₄₅-(K₁WK₁)₅ at pH 6.8 was added to the confocal dish. Imaging was performed after incubation for a certain period.

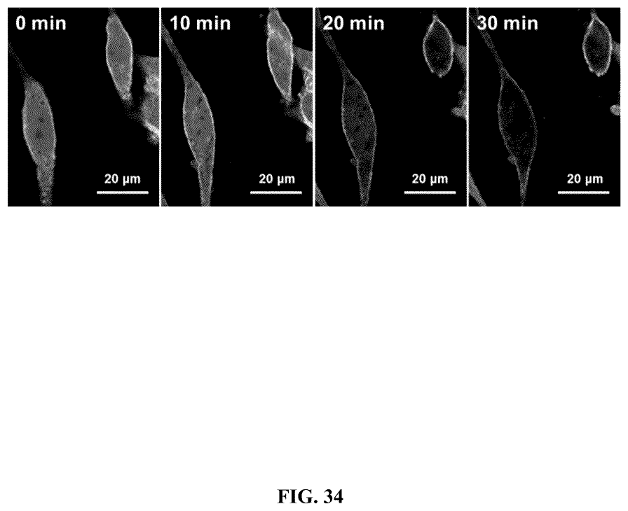

As shown in FIG. 34, red represents a mCherry protein, green represents a GFP protein, and purple represents Cy5-labeled mPEG₄₅-(K₁WK₁)₅. At pH 6.8, mPEG₄₅-(K₁WK₁)₅ causes leakage of the cytoplasmic mCherry protein.

2) Killing of mPEG₄₅-(K₁WK₁)₅ on MC38 cells: the MC38 cells were seeded into a confocal dish at a density of 100,000 cells per well. The cells were cultured for 24 h in the 37° C., CO₂ incubator. 1 mL of a drug solution containing 400 g/mL mPEG₄₅-(K₁WK₁)₅ at pH 6.8 was prepared. 5 μL of Annexin V-FITC and 1 μL of propidium iodide (PI) were added to the drug solution and mixed well. After adjusting the parameters on the instrument, the supernatant in the confocal dish was replaced with the solution described above. Imaging was performed after incubation for a certain period.

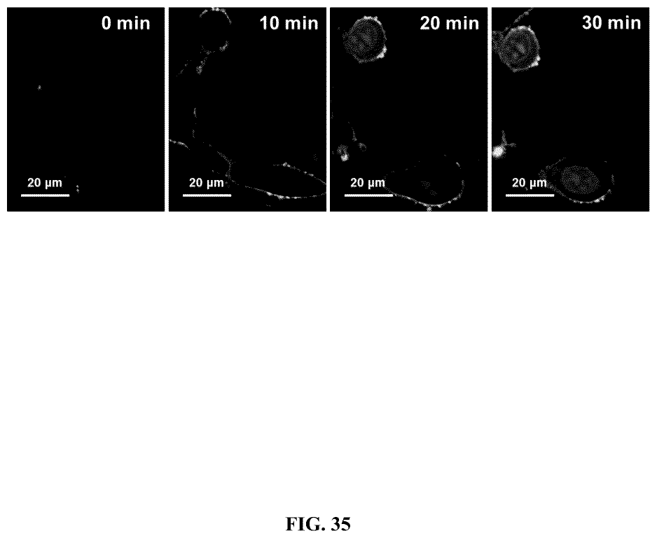

As shown in FIG. 35, green represents Annexin V-FITC, which binds to negatively charged phosphatidylserine (PS) on the cell membrane and is used to label the cell membrane; red represents Cy5-labeled mPEG₄₅-(K₁WK₁)₅; and blue represents PI, a membrane-impermeable dye used to stain nuclei of dead cells. As shown in FIG. 35, it is observed that the mPEG₄₅-(K₁WK₁)₅ material is bound to the cell membrane, and the cells quickly undergo bubbling and died. At this time, PI enters the cells, indicating cell death.

These data indicate that mPEG₄₅-(K₁WK₁)₅ causes cell membrane disruptive death by disrupting the cell membrane.

### Example 2: Sequence-Defined Polypeptides of Different Tertiary Amines-Modified methoxy poly(ethylene glycol)-lysine and tryptophan

R—OH was diethylaminoethanol (DE), N-hydroxyethylpiperidine (C₆), 2-(hexamethyleneimino)ethanol (C₇), N-benzyl-4-hydroxypiperidine (C₆P), N-methyl-N-hydroxyethylbenzylamine (MP), N-benzyl-L-prolinol (C₅P), or (S)-3-hydroxy-1-benzylpyrrolidine (C₅P₂). R₂ was a tryptophan residue, and y was 45. mPEG₄₅-COOH was reacted with an amino group in the (K)ₙ(W)ₘ sequence-defined polypeptide to obtain mPEG₄₅-(K)ₙ(W)ₘ. The tertiary amines were modified onto side chains of lysine via CDI, yielding tertiary amine-modified sequence-defined polypeptides. The effects of different tertiary amine modifications on pKa and pH-selective killing of the polypeptides were tested.

### (I) Preparation of R—OH Modified Sequence-Defined Polypeptides

A reaction scheme is as follows:

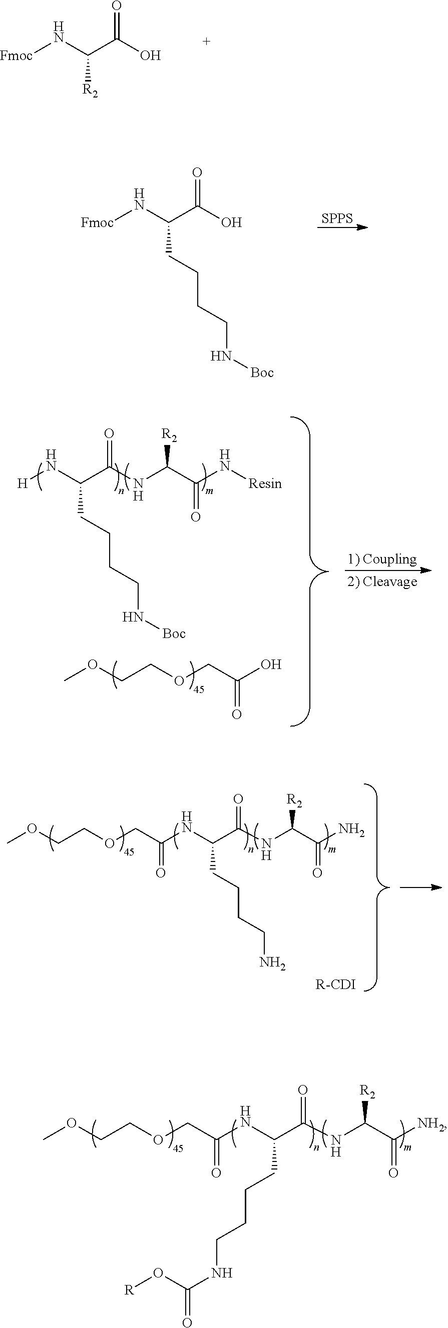

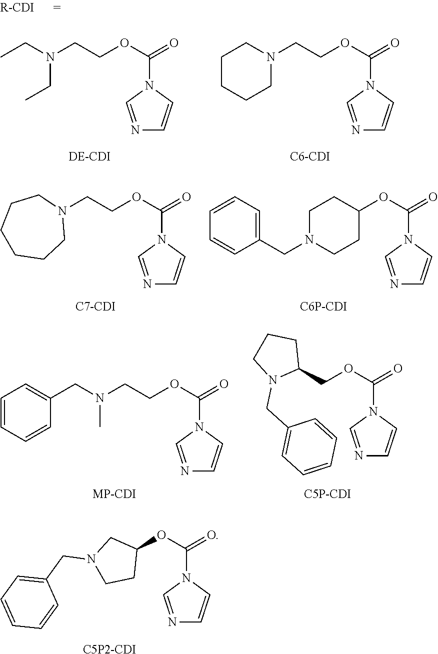

Specific steps were as follows:

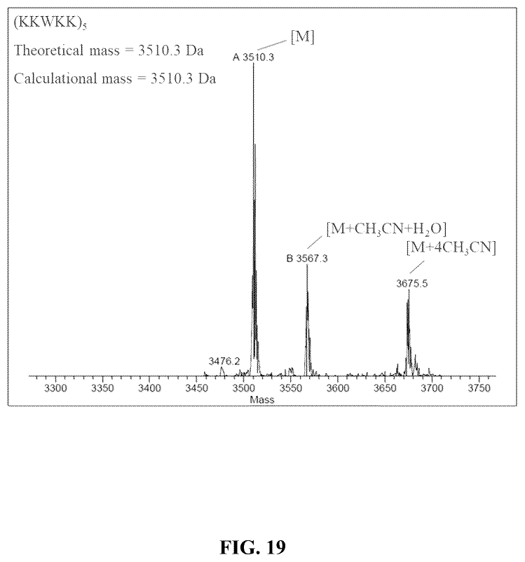

(1) H-[Lys(Boc)]i[Trp(Boc)]ₘ-Resin was synthesized using the SPPS. After each amino acid coupling, a ninhydrin reagent was used to detect whether an amino group reaction was complete. A portion of the resin was cleaved using a cleavage solution composed of 95% TFA, 2.5% H₂O, and 2.5% TIS. The product was characterized by mass spectrometry to confirm a related structure. As shown in FIG. 19, the results indicates that a polypeptide with a sequence (KKWKK)₅ is obtained.

(2) mPEG₄₅-[Lys(Boc)]4[Trp(Boc)]ₘ-Resin was synthesized using the SPPS. After the reaction was complete, a completeness of an amino group reaction was tested using a ninhydrin reagent.

(3) mPEG₄₅-[Lys(Boc)]i[Trp(Boc)]ₘ sequence-defined polypeptide was cleaved for 3 h using the cleavage solution composed of 95% TFA, 2.5% H₂O, and 2.5% TIS. The cleavage solution was concentrated with nitrogen gas and then precipitated into ice-cold ether. The precipitate was obtained by centrifugation, dissolved in deionized water, loaded into a 3500 Da dialysis bag, and dialyzed in the deionized water for 24 h, and the deionized water was replaced every 2 h. After lyophilization, a white powder of mPEG₄₅-(K)ₙ(W)ₘ sequence-defined polypeptide was obtained. A specific sequence is mPEG₄₅-KKWKKKKWKKKKWKKKKWKKKKWKK, which is referred to as mPEG₄₅-(KKWKK)₅.

(4) The CDI was placed in a round-bottom flask. Anhydrous dichloromethane was added for stirring and dispersion (5 mL dichloromethane for 1.0 g CDI). The round-bottom flask was sealed with a rubber stopper. The R—OH (3-fold excess relative to CDI) was slowly added using a syringe. The solution gradually became clear. After reacting for 12 h, an equal volume of the deionized water relative to the dichloromethane was added. The stirring was continued until no bubbles emerged. A lower dichloromethane phase was collected using a separatory funnel. Anhydrous magnesium sulfate was added for drying for 2 h. The solid was removed by filtration. The obtained solution was concentrated under reduced pressure and dried, yielding R-CDI.

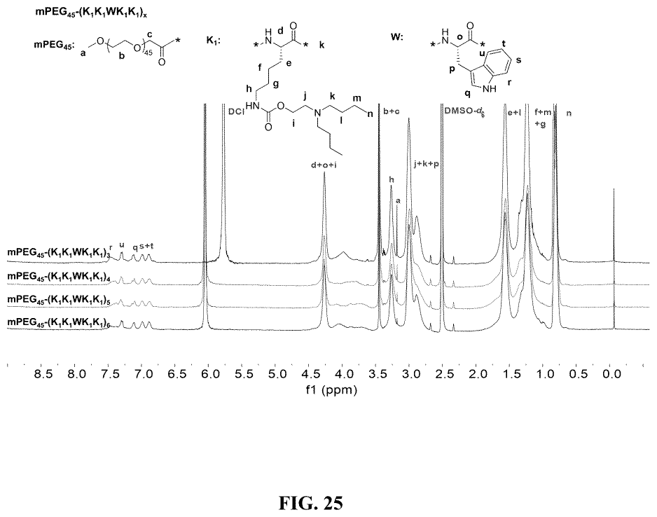

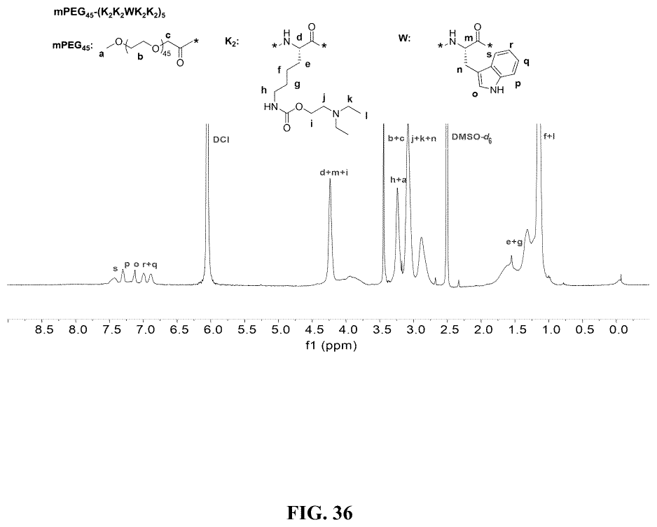

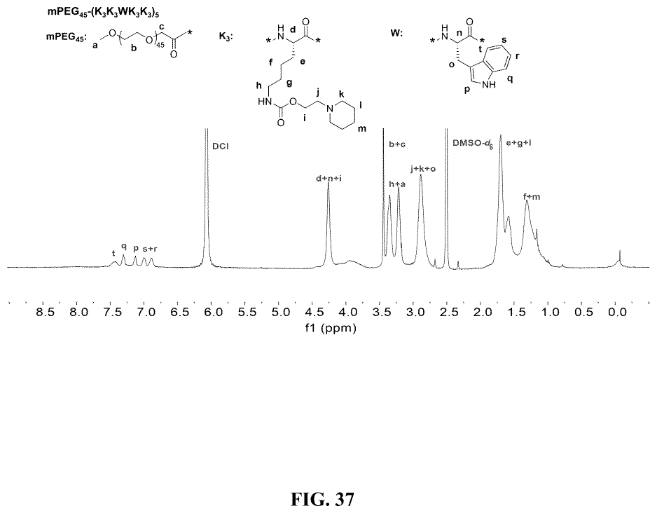

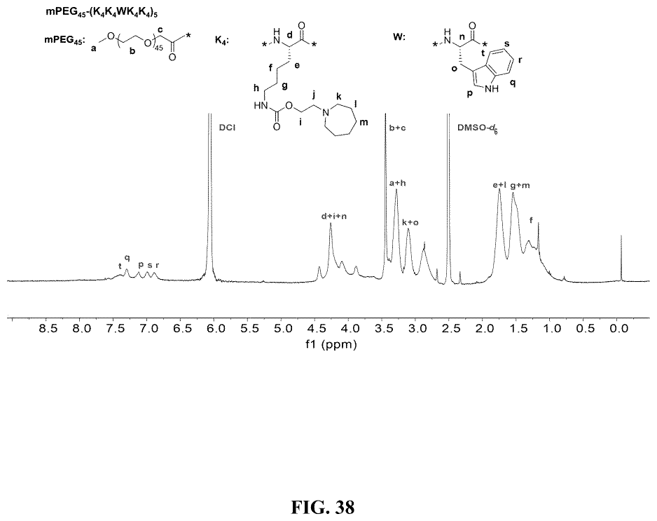

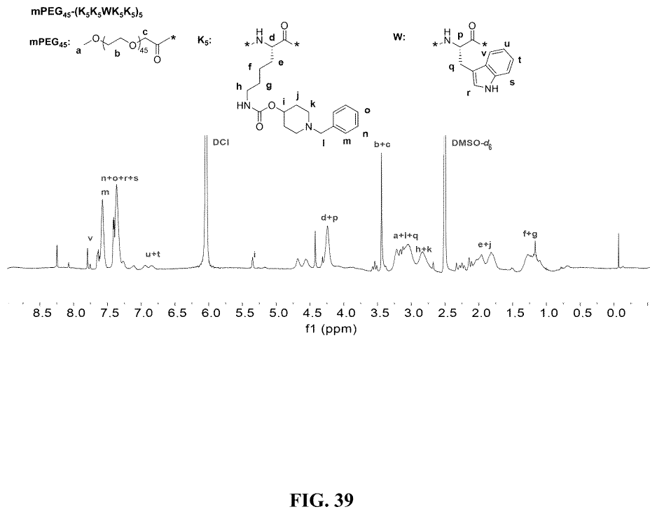

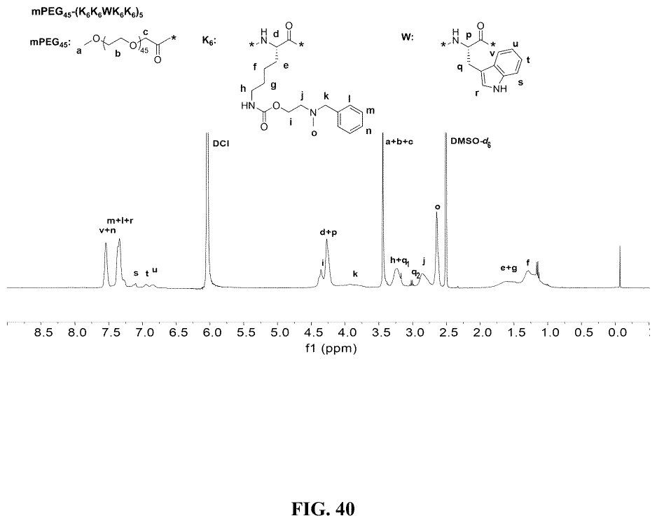

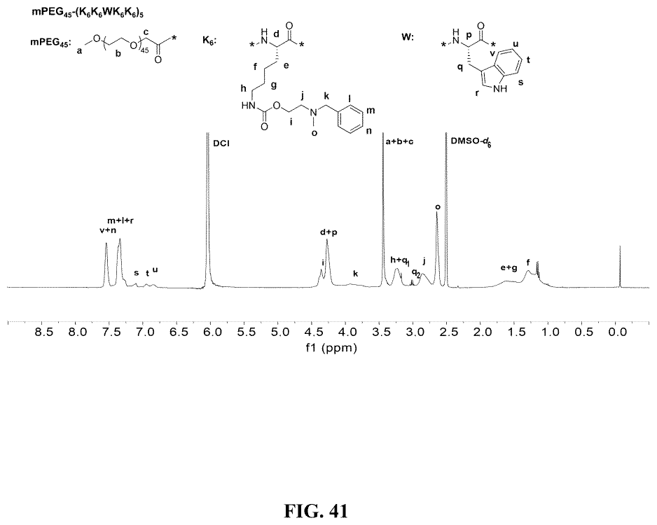

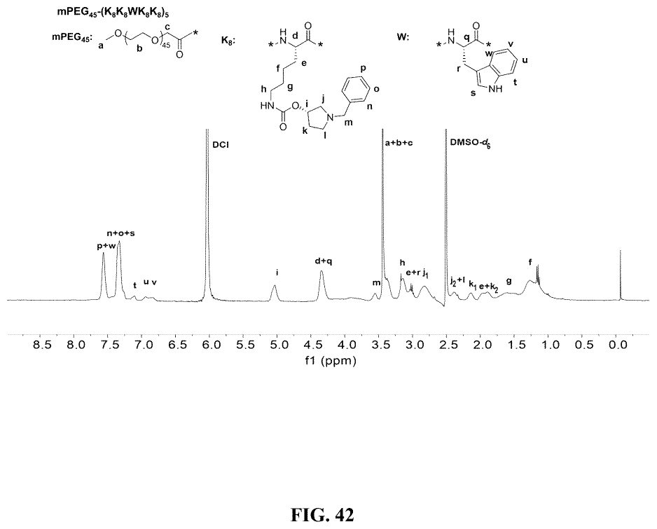

(5) The mPEG₄₅-(KKWKK)₅ sequence-defined polypeptide was dissolved in DMF, and the R-CDI (5-fold excess) and triethylamine (an equimolar to lysine side chain amino groups) were added. The reaction proceeded for 12 h. The solvent was removed using an oil pump. The crude product was dissolved in DMSO. The solution was loaded into a 3.5 kDa dialysis bag and dialyzed in the deionized water for 24 h, and the deionized water was replaced every 2 h. After lyophilization, sequence-defined polypeptides modified with the different tertiary amines were obtained. The DB-modified lysine is denoted as K₁, the DE-modified lysine is denoted as K₂, the C6-modified lysine is denoted as K₃, the C7-modified lysine is denoted as K₄, the C6P-modified lysine is denoted as K₅, the MP-modified lysine is denoted as K₆, the C₅P-modified lysine is denoted as K₇, and the C₅P₂-modified lysine is denoted as K₅. Accordingly, the tertiary amine-modified sequence-defined polypeptides obtained in this example are referred to as mPEG₄₅-(K₁K₁WK₁K₁)₅, mPEG₄₅-(K₂K₂WK₂K₂)₅, mPEG₄₅-(K₃K₃WK₃K₃)₅, mPEG₄₅-(K₄K₄WK₄K₄)₅, mPEG₄₅-(K₅K₅WK₅K₅)₅, mPEG₄₅-(K₆K₆WK₆K₆)₅, mPEG₄₅-(K₇K₇WK₇K₇)₅, and mPEG₄₅-(K₈K₈WK₈K₈)₅. Structures were characterized by proton nuclear magnetic resonance (¹H NMR) spectra (FIG. 25, FIG. 36 to FIG. 42). It can be seen that a peak corresponding to a methylene group of a lysine side chain connected to a primary amine disappears, proving complete coupling. For an MP-modified polypeptide, i.e., mPEG₄₅-(K₆K₆WK₆K₆)₅, an integral ratio of a signal peak for hydrogen atoms of the methylene group at h-position on the lysine side chain to a signal peak for the hydrogen atoms of a methyl group at o-position of tertiary amine MP is approximately 2:3, indicating that mPEG₄₅-(KKWKK)₅ is completely modified by the tertiary amine MP, i.e., mPEG₄₅-(K₆K₆WK₆K₆)₅ is successfully synthesized.

Structural formulas of the sequence-defined polypeptides prepared in this example are as follows:

| | |
|---|---|
| mPEG₄₅-(K₁K₁WK₁K₁)₅ | mPEG₄₅-(K₂K₂WK₂K₂)₅ |
| 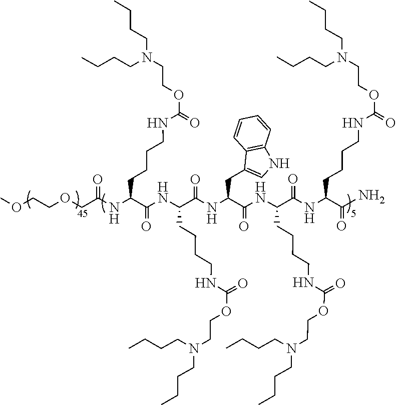 | 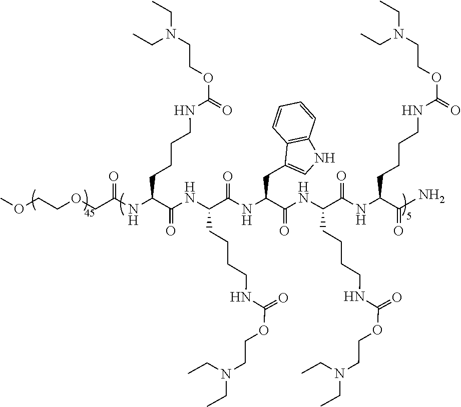 |
| mPEG₄₅-(K₃K₃WK₃K₃)₅ | mPEG₄₅-(K₄K₄WK₄K₄)₅ |
| 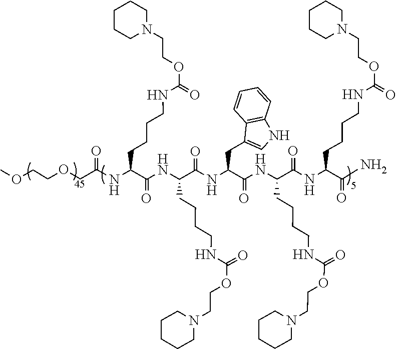 | 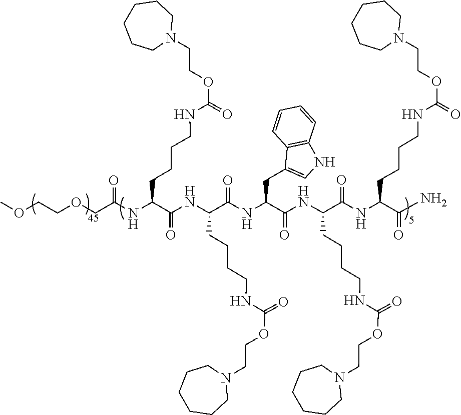 |
| mPEG₄₅-(K₅K₅WK₅K₅)₅ | mPEG₄₅-(K₆K₆WK₆K₆)₅ |
| 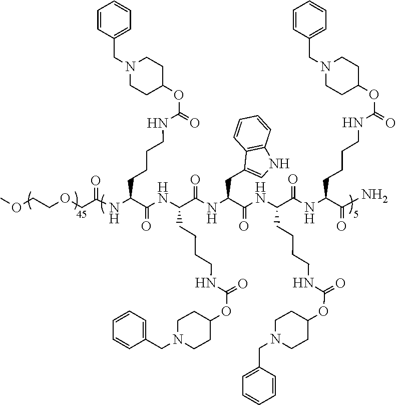 | 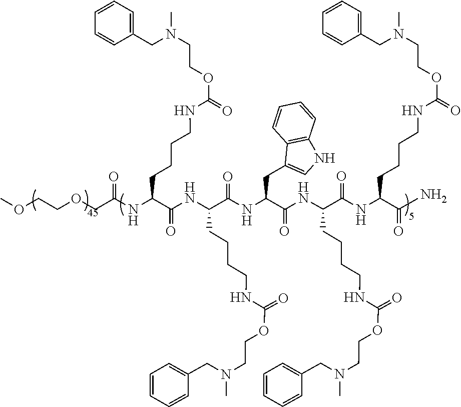 |
| mPEG₄₅-(K₇K₇WK₇K₇)₅ | mPEG₄₅-(K₈K₈WK₈K₈)₅ |
| 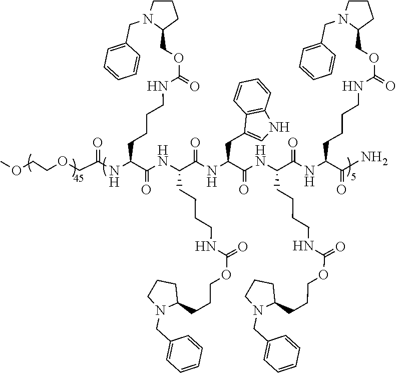 | 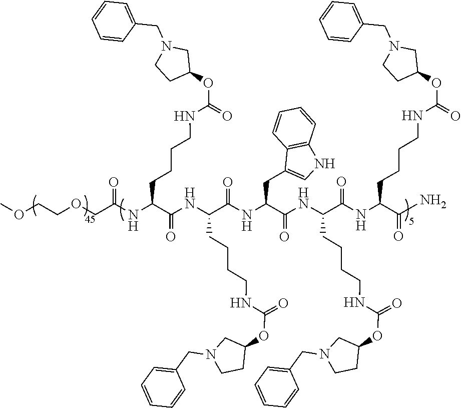 |

### (II) Effects of Different Tertiary Amine Modifications on Lysine Side Chains on pKa of Polypeptide

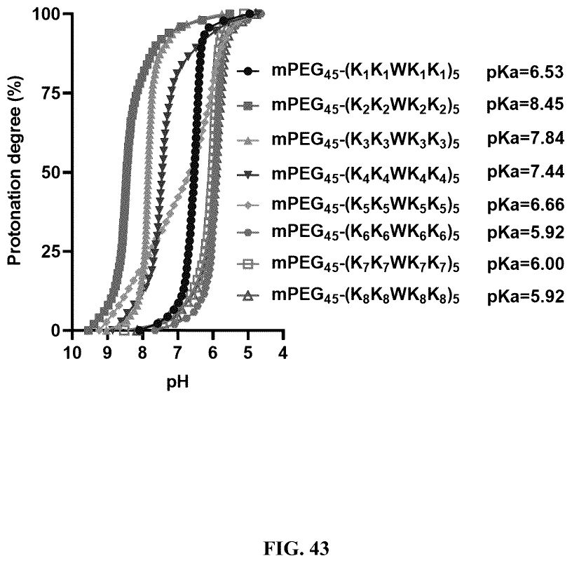

100 μL of concentrated hydrochloric acid (37%, 12 mol) was added to 100 mL of the deionized water and fully mixed to obtain a clear and transparent solution. 10 mg of the sequence-defined polypeptide material prepared in step (5) was dissolved in 10 mL of the hydrochloric acid solution. A pH probe was immersed below a liquid surface. Under a stirring condition (a stirring speed Mot of 3), a titration was performed using a 0.5 M sodium hydroxide titrant. After a pH meter stabilized, a reading was recorded. The titration continued until the reading reached pH=11. A derivative of a titration curve was taken to obtain extremum points, which were defined as points of protonation degrees of 1 and 0, respectively, thereby establishing a pH-protonation degree curve. According to a definition of a dissociation equilibrium constant (pKa), a pH corresponding to a protonation degree of 0.5 for the polypeptide was defined as a pKa of the material. The results are shown in FIG. 43. The protonation degree of this type of polypeptides exhibits a sharp transition with changing pH. As hydrophobicity of the tertiary amine decreases, the pKa of the polypeptides shows an increasing trend.

### (III) Cytotoxicity of Sequence-Defined Polypeptides at Different pH Values

10 mg of the polypeptide prepared in step (5) was placed in a 5 mL sample vial. 0.3 mL of DMF was added for fully dissolution for later use. 0.9 mL of sterile water was added into a pre-sterilized 25 mL round-bottom flask on a magnetic stirrer. The solution was stirred at a speed of RPM=600 r/min. A DMF solution of the polypeptide was added dropwise to the round-bottom flask using a pipette. Stirring was continued at RPM=400 r/min for 15 min. Dialysis was then performed using a dialysis bag with a molecular weight cut-off of 14000 Da in 4 μL of ultrapure water at 4° C. for 24 h. The ultrapure water was replaced every 1 h during first 6 h and every 6 h for subsequent 18 h, yielding polypeptide nanoparticles. After the dialysis, the solution was taken out using the pipette, quantified, and stored in a 4° C. refrigerator.

A killing effect of the polypeptide nanoparticles on tumor cells under pH 7.4 and pH 6.8 conditions was evaluated by a CCK-8 assay. A DMEM was adjusted to pH=6.8 with a 6 mol/L hydrochloric acid solution and reserved for later use. Mouse colorectal cancer cells MC38 (purchased from ATCC) was cultured in the DMEM containing 10% (v/v) fetal bovine serum. Under pH 7.4 and pH 6.8 conditions, cells (at a concentration of 1×10⁵ cells/mL) were incubated with different concentrations of the polypeptide nanoparticles prepared in this example. After incubation for 24 h in a 37° C. incubator, the original medium was discarded. 100 μL of a CCK-8 solution was added, and incubation continued in a 37° C., CO₂ incubator for 2 h. An absorbance at 450 nm was measured using a microplate reader.

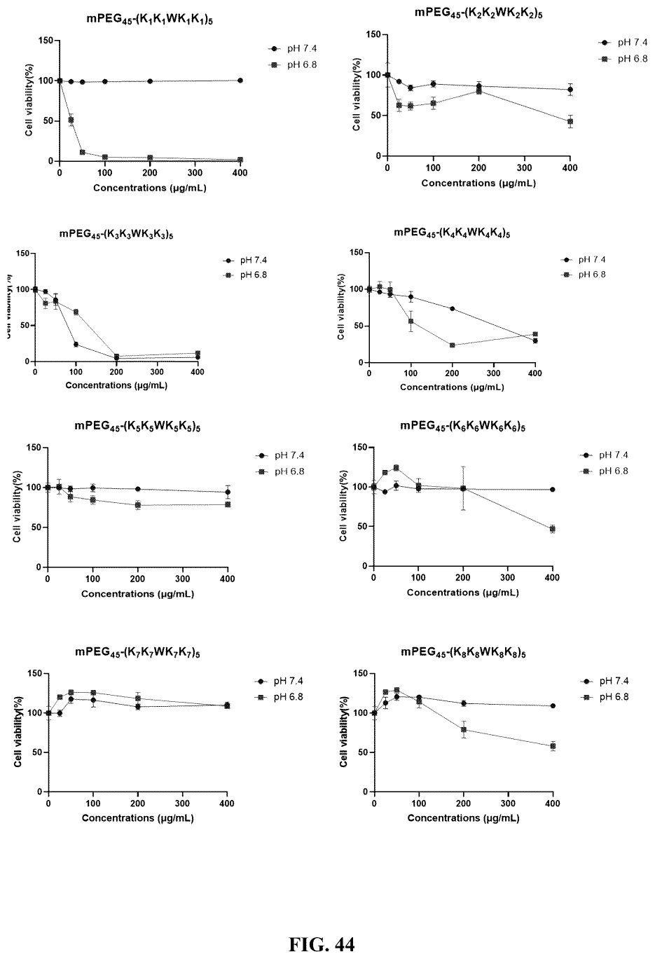

The test results are shown in FIG. 44. The sequence-defined polypeptide of DE-modified tryptophan and lysine, i.e., mPEG₄₅-(K₂K₂WK₂K₂)₅, exhibits low toxicity to the tumor cells at pH 7.4, and shows weak killing toxicity at pH 6.8, lacking pH selectivity. The sequence-defined polypeptide of C6-modified tryptophan and lysine, i.e., mPEG₄₅-(K₃K₃WK₃K₃)₅, exhibits strong killing toxicity to the tumor cells at both pH 7.4 and pH 6.8, also lacking pH selectivity. The sequence-defined polypeptide of C₆P-modified tryptophan and lysine, i.e., mPEG₄₅-(K₅K₅WK₅K₅)₅, does not cause the tumor cell death at pH 7.4, and exhibits weak killing toxicity at pH 6.8, lacking pH selectivity. The sequence-defined polypeptide of MP-modified tryptophan and lysine, i.e., mPEG₄₅-(K₆K₆WK₆K₆)₅, does not cause the tumor cell death at pH 7.4, and exhibits weak killing toxicity at pH 6.8, lacking pH selectivity. The sequence-defined polypeptide of C₅P-modified tryptophan and lysine, i.e., mPEG₄₅-(K₇K₇WK₇K₇)₅, does not cause the tumor cell death at either pH 7.4 or 6.8, lacking pH selectivity. The sequence-defined polypeptide of C₅P₂-modified tryptophan and lysine, i.e., mPEG₄₅-(K₈K₈WK₈K₈)₅, does not cause the tumor cell death at pH 7.4, but exhibits certain killing toxicity at pH 6.8, showing certain pH-selective killing toxicity. The sequence-defined polypeptide of C7-modified tryptophan and lysine, i.e., mPEG₂ₖ-(K₄K₄WK₄K₄)₅, exhibits better tumor cell cytotoxicity at pH 6.8 than that at pH 7.4, showing certain pH-selective killing toxicity. However, both mPEG₄₅-(K₈K₈WK₈K₈)₅ and mPEG₂ₖ-(K₄K₄WK₄K₄)₅ exhibit lower selectivity compared to the sequence-defined polypeptide of DB-modified tryptophan and lysine (mPEG₄₅-(K₁K₁WK₁K₁)₅). Thus, for the sequence-defined polypeptides of a same sequence, the different tertiary amine modifications exhibit different tumor cell killing effects.

### Example 3: Sequence-Defined Polypeptides of PEG of Different Lengths-Modified methoxy poly(ethylene glycol)-lysine and tryptophan

R—OH was 2-(dibutylamino)ethanol (DB), R₂ was a tryptophan residue, and y was selected from 22, 45, and 113. mPEG₂₂-COOH, mPEG₄₅-COOH, and mPEG₁₁₃-COOH were reacted with an amino group in the (K)ₙ(W)ₘ sequence-defined polypeptide to obtain mPEG₂₂-(K)ₙ(W)ₘ, mPEG₄₅-(K)ₙ(W)ₘ, and mPEG₁₁₃-(K)ₙ(W)ₘ. Tertiary amines were modified onto lysine side chains via CDI, yielding tertiary amine-modified sequence-defined polypeptides. Effects of PEG with different lengths on pKa and pH-selective killing of the sequence-defined polypeptide were tested.

### (I) Preparation of 2-(dibutylamino)ethanol-Modified Sequence-Defined Polypeptides

A reaction scheme is as follows:

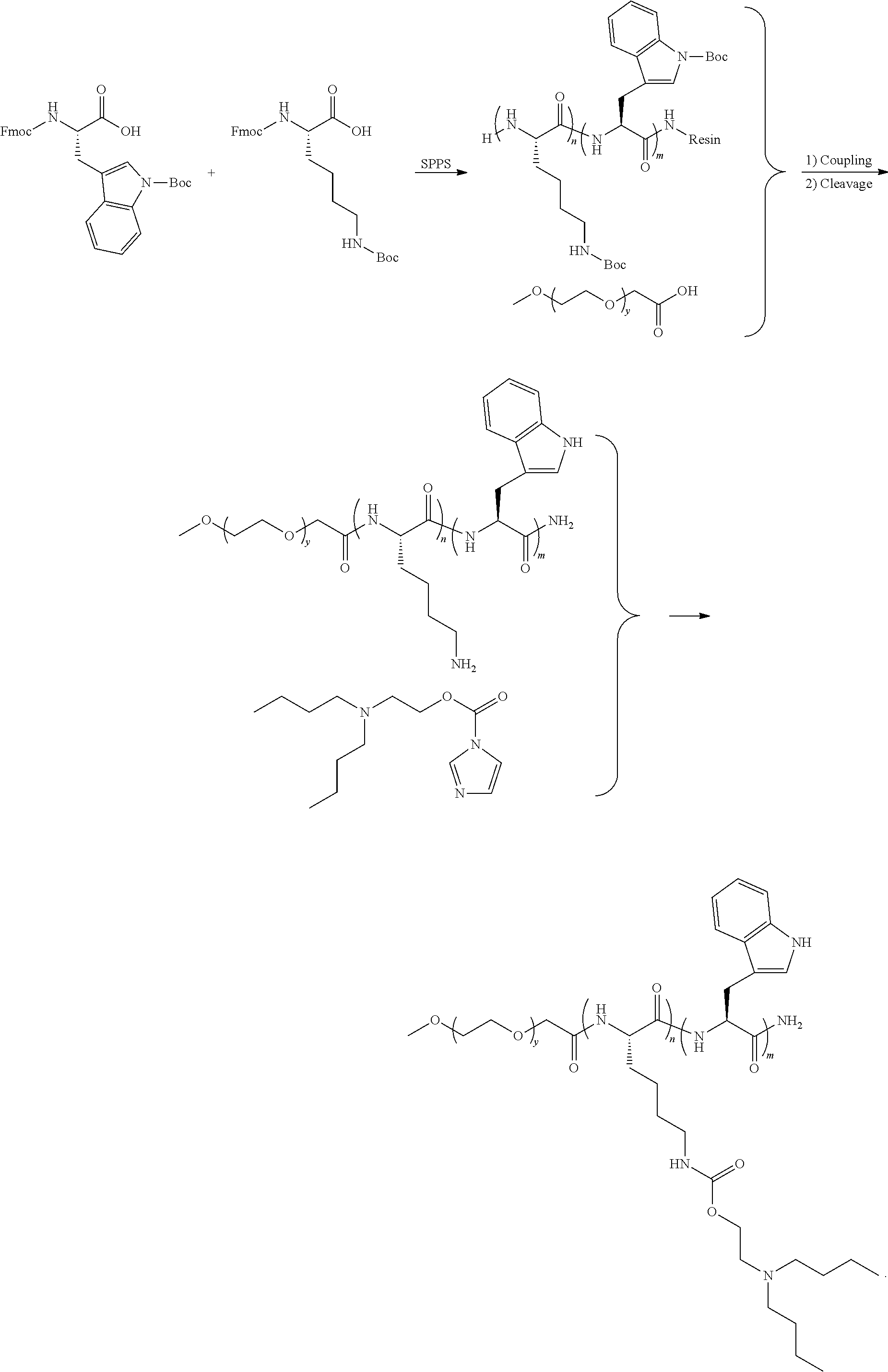

Specific steps were as follows:

(1) H-[Lys(Boc)]4[Trp(Boc)]m-Resin was synthesized by SPPS. After each amino acid coupling, a completeness of an amino group reaction was tested using a ninhydrin reagent. A portion of the resin was cleaved using a cleavage solution composed of 95% TFA, 2.5% H₂O, and 2.5% TIS. The product was characterized by mass spectrometry to confirm a related structure. The results are shown in FIG. 19, indicating that a polypeptide with a sequence (KKWKK)₅ is obtained.

(2) mPEG₂₂-[Lys(Boc)]i[Trp(Boc)]ₘ-Resin, mPEG₄₅-[Lys(Boc)]4[Trp(Boc)]ₘ-Resin, and mPEG₁₁₃-[Lys(Boc)]4[Trp(Boc)]ₘ-Resin were synthesized by the SPPS. After the reaction was complete, a completeness of an amino group reaction was tested using a ninhydrin reagent.

(3) mPEG₂₂-[Lys(Boc)]n[Trp(Boc)]ₘ-Resin, mPEG₄₅-_{[}[Lys(Boc)]4[Trp(Boc)]ₘ-Resin, and mPEG113-[Lys(Boc)]4[Trp(Boc)]ₘ-Resin sequence-defined polypeptides were cleaved for 3 h using the cleavage solution composed of 95% TFA, 2.5% H₂O, and 2.5% TIS. The cleavage solution was concentrated with nitrogen gas and precipitated into ice-cold ether. The precipitate was obtained by centrifugation, dissolved in deionized water, loaded into a 3500 Da dialysis bag, and dialyzed in deionized water for 24 h, and the deionized water was replaced every 2 h. After lyophilization, white powders of mPEG₂₂-(K)ₙ(W)ₘ, mPEG₄₅-(K)ₙ(W)ₘ, and mPEG₁₁₃-(K)ₙ(W)ₘ sequence-defined polypeptides were obtained. Specific sequences are mPEG₂₂-KKWKKKKWKKKKWKKKKWKKKKWKK, mPEG₄₅-KKWKKKKWKKKKWKKKKWKKKKWKK, and mPEG₁₁₃-KKWKKKKWKKKKWKKKKWKKKKWKK, which are referred to as mPEG₂₂-(KKWKK)₅, mPEG₄₅-(KKWKK)₅, and mPEG₁₁₃-(KKWKK)₅.

(4) The CDI was placed in a round-bottom flask. Anhydrous dichloromethane was added for stirring and dispersion (5 mL dichloromethane for 1.0 g CDI). The round-bottom flask was sealed with a rubber stopper. DB-OH (3-fold excess relative to the CDI) was slowly added using a syringe. The solution gradually became clear. After reacting for 12 h, an equal volume of the deionized water relative to the dichloromethane was added. Stirring was continued until no bubbles emerged. A lower dichloromethane phase was collected using a separatory funnel. Anhydrous magnesium sulfate was added for drying for 2 h. The solid was removed by filtration. The obtained solution was concentrated under reduced pressure and dried, yielding DB-CDI.

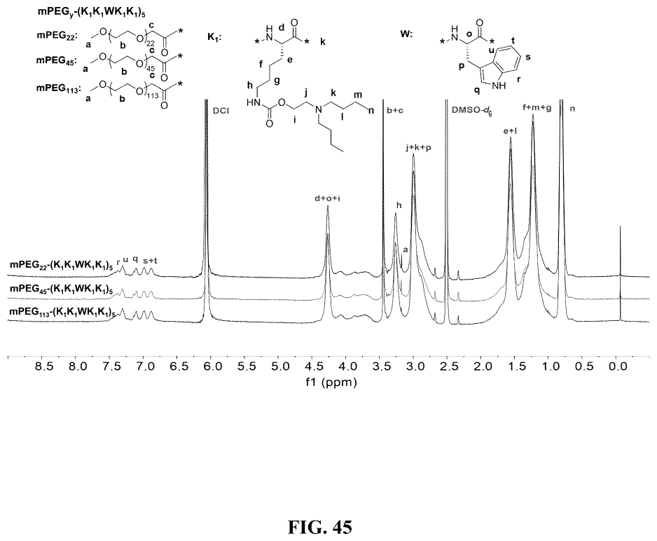

(5) The mPEG₂₂-(KKWKK)₅, mPEG₄₅-(KKWKK)₅, and mPEG₁₁₃-(KKWKK)₅ sequence-defined polypeptides were dissolved in DMF, and R-CDI (5-fold excess) and triethylamine (an equimolar to lysine side chain amino groups) were added. The reaction proceeded for 12 h. The solvent was removed using an oil pump. The crude product was dissolved in DMSO. Dialysis was performed using a 3.5 kDa dialysis bag in the deionized water for 24 h, and the deionized water was replaced every 2 h. After lyophilization, sequence-defined polypeptides modified with the PEG of different lengths were obtained. The DB-modified lysine is denoted as K₁, accordingly, the sequence-defined polypeptides modified with PEG of different lengths obtained in this example are referred to as mPEG₂₂-(K₁K₁WK₁K₁)₅, mPEG₄₅-(K₁K₁WK₁K₁)₅, and mPEG₁₁₃-(K₁K₁WK₁K₁)₅. Structures were characterized by proton nuclear magnetic resonance (¹H NMR) spectra (as shown in FIG. 45). It can be seen that a peak corresponding to a methylene group of the lysine side chain connected to a primary amine group disappears, proving complete bonding.

Structural formulas of the sequence-defined polypeptides prepared in this example are as follows:

| | |
|---|---|
| mPEG₂₂-(K₁K₁WK₁K₁)₅ | mPEG₄₅-(K₁K₁WK₁K₁)₅ |
| 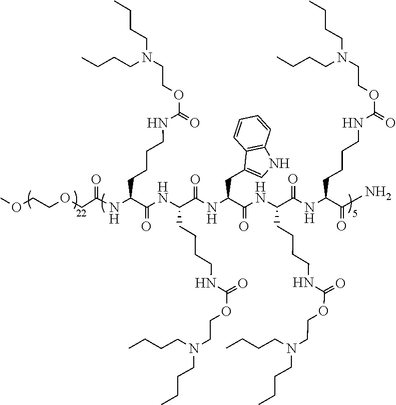 | 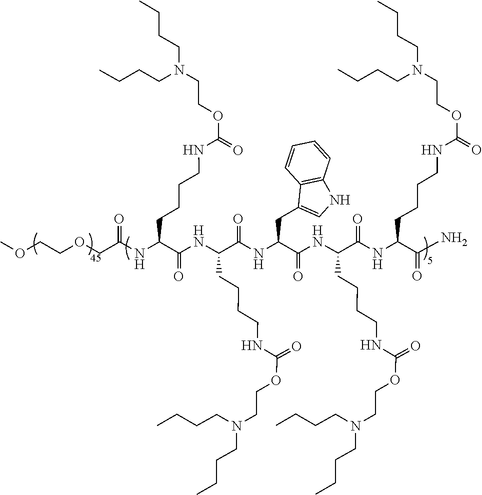 |
| mPEG₁₁₃-(K₁K₁WK₁K₁)₅ | |

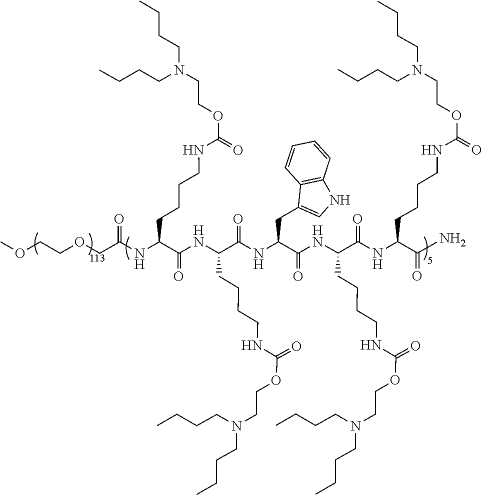

(II) Effects of PEG with Different Lengths on pKa of Polypeptides

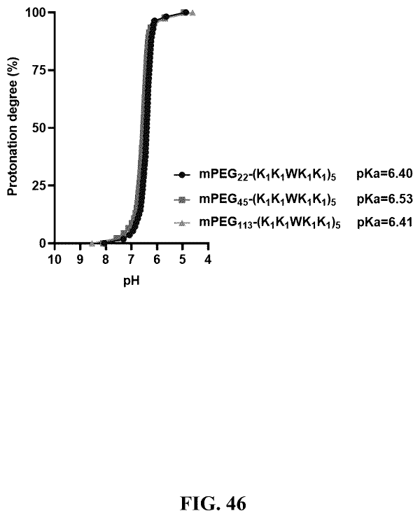

100 μL of concentrated hydrochloric acid (3700, 12 mol) was added to 100 mL of the deionized water and fully mixed to obtain a clear and transparent solution. 10 mg of the sequence-defined polypeptide material prepared in step (5) was dissolved in 10 mL of the hydrochloric acid solution, and a pH probe was immersed below a liquid surface. Under a stirring condition (a stirring speed Mot of 3), a titration was performed using a 0.5 M sodium hydroxide titrant. After a pH meter stabilized, a reading was recorded. The titration continued until the reading reached pH=11. A derivative of a titration curve was taken to obtain extremum points, which were defined as points of protonation degrees of 1 and 0, respectively, thereby establishing a pH-protonation degree curve. According to a definition of a dissociation equilibrium constant (pKa), a pH corresponding to a protonation degree of 0.5 for the polypeptide was defined as a pKa of the material. The results are shown in FIG. 46. The protonation degree of this type of the polypeptides exhibits a sharp transition with changing pH, and the length of the PEG have little effect on the pKa of the material.

### (III) Cytotoxicity of Sequence-Defined Polypeptides at Different pH Values

10 mg of the polypeptide prepared in step (5) was placed in a 5 mL sample vial. 0.3 mL of DMF was added for fully dissolution for later use. 0.9 mL of sterile water was added into a pre-sterilized 25 mL round-bottom flask on a magnetic stirrer. The solution was stirred at a speed of RPM=600 r/min. A DMF solution of the polypeptide was added dropwise to the round-bottom flask using a pipette. Stirring was continued at RPM=400 r/min for 15 min. Dialysis was then performed using a dialysis bag with a molecular weight cut-off of 14000 Da in 4 μL of ultrapure water at 4° C. for 24 h. The ultrapure water was replaced every 1 h during first 6 h and every 6 h for subsequent 18 h, yielding polypeptide nanoparticles. After the dialysis, the solution was taken out using the pipette, quantified, and stored in a 4° C. refrigerator.

A killing effect of the polypeptide nanoparticles on tumor cells under pH 7.4 and pH 6.8 conditions was evaluated by a CCK-8 assay. A DMEM was adjusted to pH=6.8 with a 6 mol/L hydrochloric acid solution and reserved for later use. Mouse colorectal cancer cells MC38 (purchased from ATCC) were cultured in a DMEM containing 10% (v/v) fetal bovine serum. Under pH 7.4 and pH 6.8, cells (at a concentration of 1×10⁵ cells/mL) were incubated with different concentrations of the polypeptide nanoparticles prepared in this example. After incubation for 24 h in a 37° C. incubator, the original medium was discarded. 100 μL of a CCK-8 solution was added, and incubation continued in a 37° C., CO₂ incubator for 2 h. An absorbance at 450 nm was measured using a microplate reader.

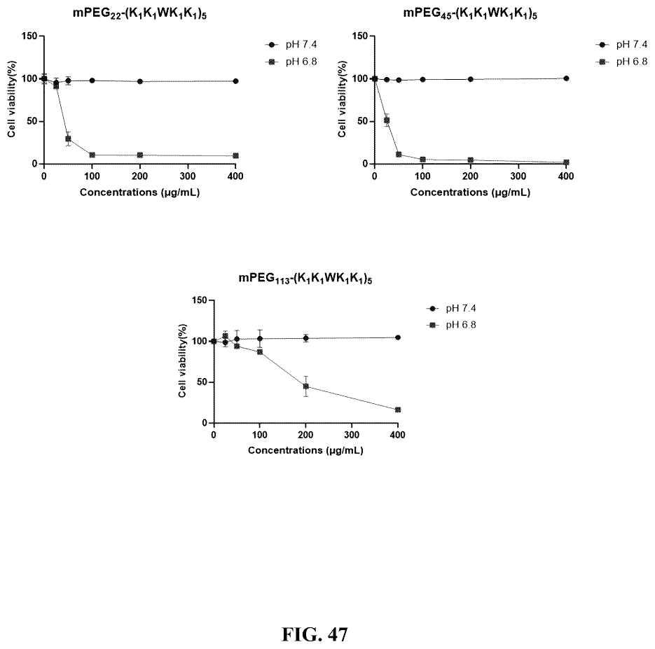

The test results are shown in FIG. 47. The sequence-defined polypeptides of DB-modified PEG of different lengths all exhibit low cytotoxicity to the tumor cells at pH 7.4, but show strong tumor cell cytotoxicity at pH 6.8, indicating a good selective killing effect on the tumor cells. When a PEG molecular weight is about 5000, i.e., y is 113, the tumor cell cytotoxicity of the polypeptide at pH 6.8 is attenuated.

### Example 4: Sequence-Defined Polypeptide of 2-(dibutylamino)ethanol-Modified methoxy poly(ethylene glycol)-lysine and tryptophan

R—OH was 2-(dibutylamino)ethanol (DB), R₂ was a tryptophan residue, and y was 45. mPEG₄₅-COOH was reacted with an amino group in (K)ₙ(W)ₘ sequence-defined polypeptide to obtain mPEG₄₅-(K)ₙ(W)ₘ. A tertiary amine was modified onto a side chain of lysine via CDI to obtain a tertiary amine-modified sequence-defined polypeptide. Effects of different arrangements of amino acids on pKa and pH-selective killing of the sequence-defined polypeptide was tested.

### (I) Preparation of 2-(dibutylamino)ethanol-Modified Sequence-Defined Polypeptides

A reaction scheme is as follows:

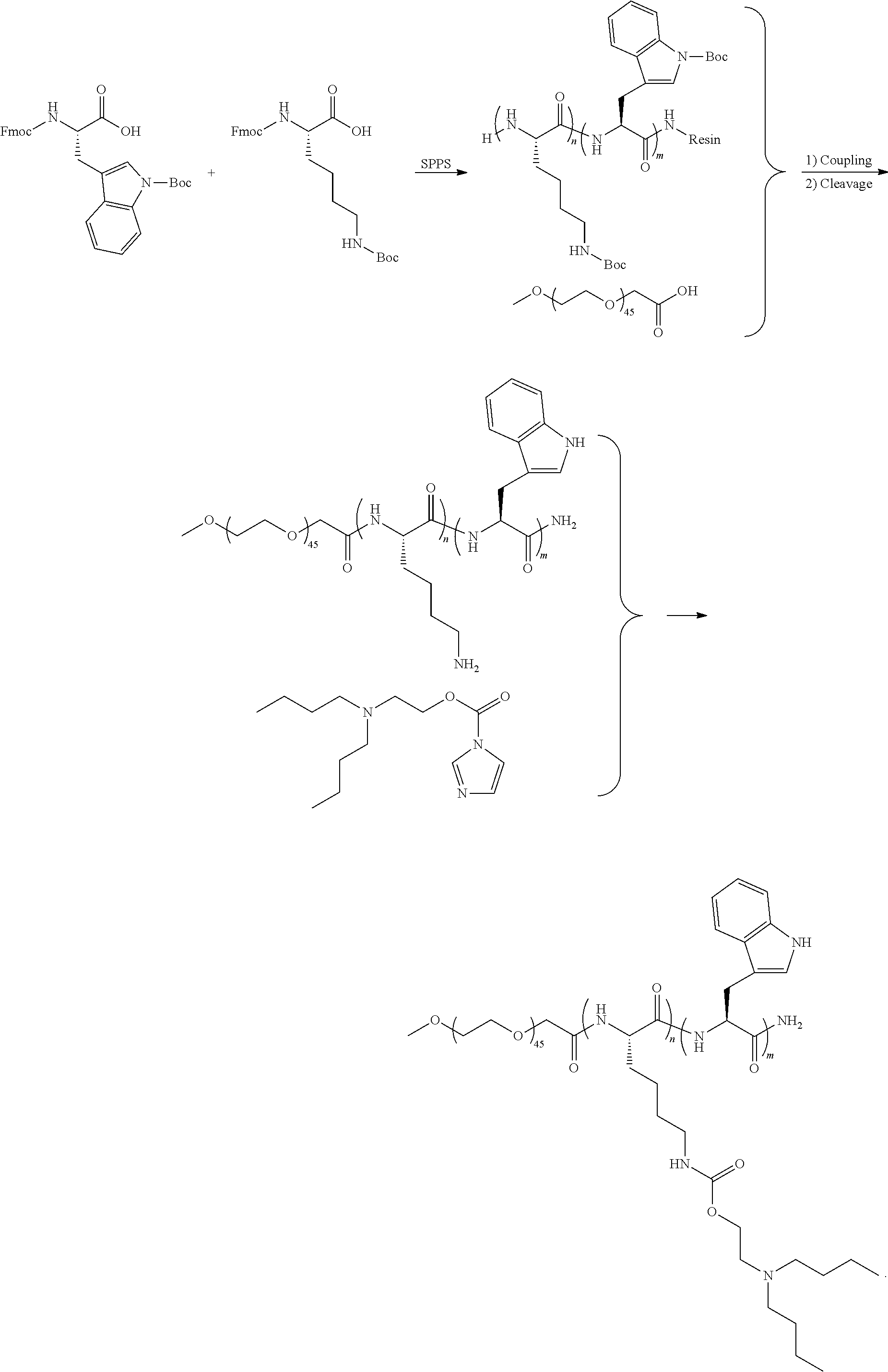

Specific steps were as follows:

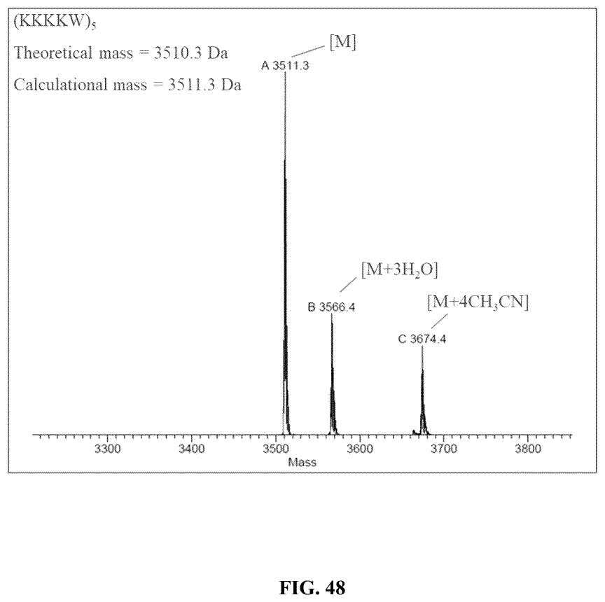

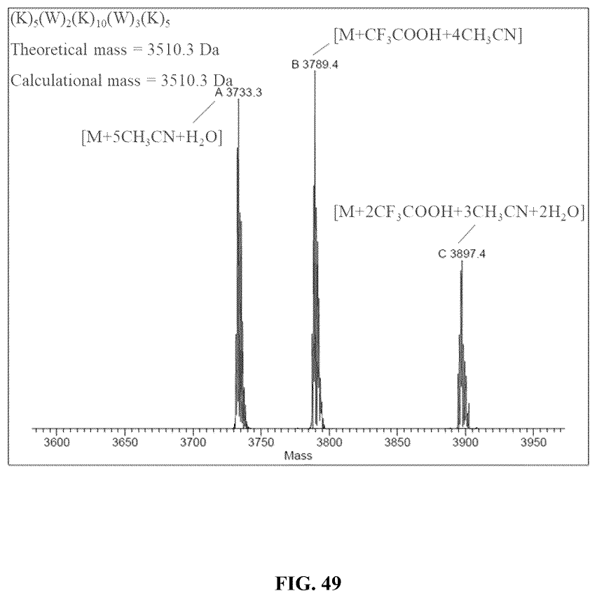

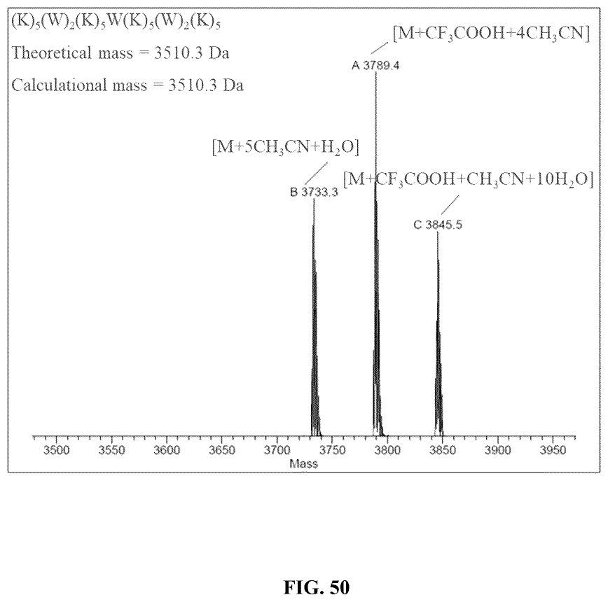

(1) H-[Lys(Boc)]4[Trp(Boc)]ₘ-Resin was synthesized by SPPS. After each amino acid coupling, a completeness of an amino group reaction was tested using a ninhydrin reagent. A portion of the resin was cleaved using a cleavage solution composed of 95% TFA, 2.5% H₂O, and 2.5% TIS. The product was characterized by mass spectrometry to confirm a related structure. The results are shown in FIG. 19 and FIGS. 48-50, indicating that polypeptides with sequences of KKWKKKKWKKKKWKKKKWKKKKWKK, KKKKWKKKKWKKKKWKKKKWKKKKW, KKKKKWWKKKKKKKKKKWWWKKKKK, and KKKKKWWKKKKKWKKKKKWWKKKKK are obtained.

(2) mPEG₄₅-[Lys(Boc)]_{n[}[Trp(Boc)]ₘ-Resin was synthesized by the SPPS, and after the reaction was complete, the ninhydrin reagent was used to detect whether the amino group reaction was complete.

(3) mPEG₄₅-[Lys(Boc)]4[Trp(Boc)]ₘ sequence-defined polypeptide was cleaved for 3 h using the cleavage solution composed of 95% TFA, 2.5% H₂O, and 2.5% TIS. The cleavage solution was concentrated with nitrogen gas and precipitated into ice-cold ether. The precipitate was obtained by centrifugation, dissolved in deionized water, loaded into a 3500 Da dialysis bag, and dialyzed in the deionized water for 24 h, and the deionized water was replaced every 2 h. After lyophilization, a white powder of mPEG₄₅-(K)ₙ(W)ₘ sequence-defined polypeptides was obtained. Specific sequences are mPEG₄₅-KKWKKKKWKKKKWKKKKWKKKKWKK, mPEG₄₅-KKKKWKKKKWKKKKWKKKKWKKKKW, mPEG₄₅-KKKKKWWKKKKKKKKKKWWWKKKKK, and mPEG₄₅-KKKKKWWKKKKKWKKKKKWWKKKKK, which are referred to as mPEG₄₅-(KKWKK)₅, mPEG₄₅-(KKKKW)₅, mPEG₄₅-(K)₅(W)₂(K)₁₀(W)₃(K)₅, and mPEG₄₅-(K)₅(W)₂(K)₅W(K)₅(W)₂(K)₅, respectively.

(4) The CDI was placed in a round-bottom flask. Anhydrous dichloromethane was added for stirring and dispersion (5 mL dichloromethane for 1.0 g CDI). The round-bottom flask was sealed with a rubber stopper. 2-(dibutylamino)ethanol (3-fold excess relative to the CDI) was slowly added using a syringe. The solution gradually became clear. After reacting for 12 h, an equal volume of the deionized water relative to the dichloromethane was added. Stirring was continued until no bubbles emerged. A lower dichloromethane phase was collected using a separatory funnel. Anhydrous magnesium sulfate was added for drying for 2 h. The solid was removed by filtration. The obtained solution was concentrated under reduced pressure and dried, yielding 2-(dibutylamino)ethanol-CDI (DB-CDI).

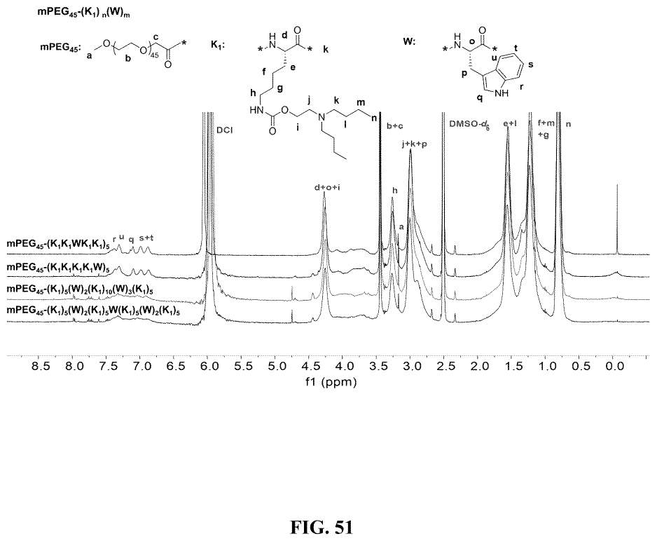

(5) The mPEG₄₅-(K)ₙ(W)ₘ sequence-defined polypeptide was dissolved in DMF, and DB-CDI (5-fold excess) and triethylamine (an equimolar to the lysine side chain amino groups) were added. The reaction proceeded for 12 h. The solvent was removed using an oil pump. The crude product was dissolved in DMSO. Dialysis was performed using a 3.5 kDa dialysis bag in the deionized water for 24 h, and the deionized water was replaced every 2 h. After lyophilization, DB-modified sequence-defined polypeptide was obtained. The DB-modified lysine is denoted as K₁, accordingly, the DB-modified sequence-defined polypeptides obtained in this example are referred to as mPEG₄₅-(K₁K₁WK₁K₁)₅, mPEG₄₅-(K₁K₁K₁K₁W)₅, mPEG₄₅-(K₁)₅(W)₂(K₁)₁₀(W)₃(K₁)₅, and mPEG₄₅-(K₁)₅(W)₂(K₁)₅W(K₁)₅(W)₂(K₁)₅. Structures were characterized by proton nuclear magnetic resonance (¹H NMR) spectra (as shown in FIG. 51). It can be seen that a peak corresponding to a methylene group of the lysine side chain connected to a primary amine group disappears, proving complete bonding.

Structural formulas of the sequence-defined polypeptides prepared in this example are as follows.

| | |
|---|---|
| mPEG₄₅-(K₁K₁WK₁K₁)₅ | mPEG₄₅-(K₁K₁K₁K₁W)₅ |
| 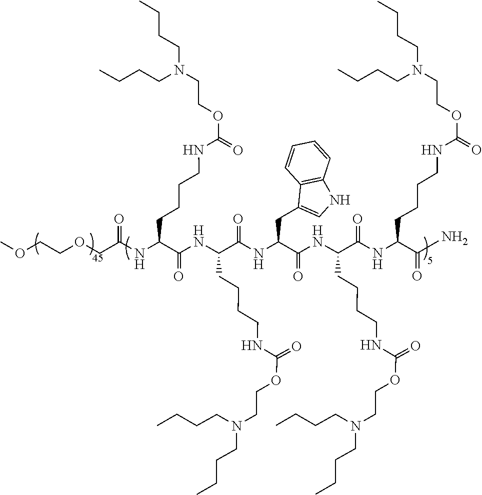 | 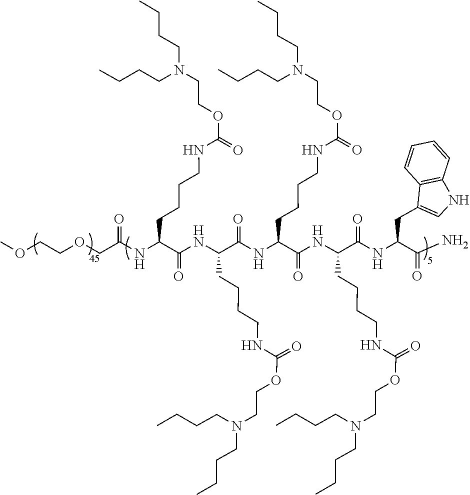 |
| mPEG₄₅-(K₁)₅(W)₂(K₁)₁₀(W)₃(K₁)₅ | mPEG₄₅-(K₁)₅(W)₂(K₁)₅W(K₁)₅(W)₂(K₁)₅ |
| 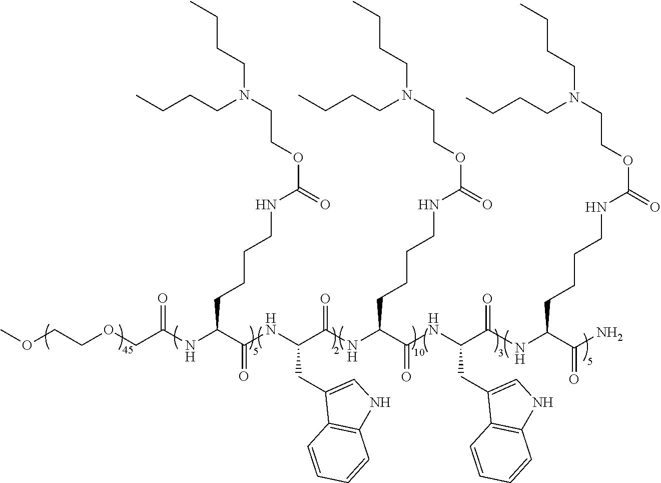 | 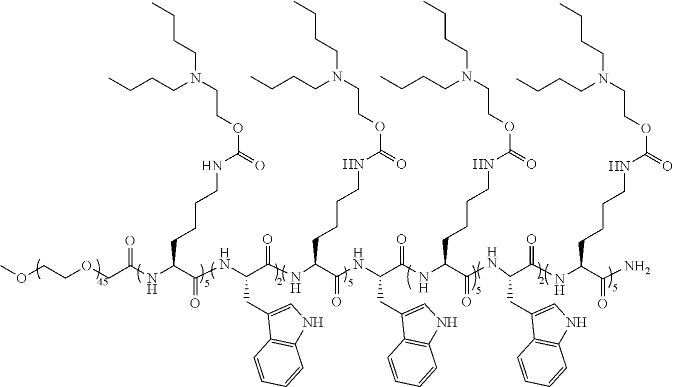 |

### (II) Effects of Different Arrangements of Amino Acids on pKa of Polypeptides

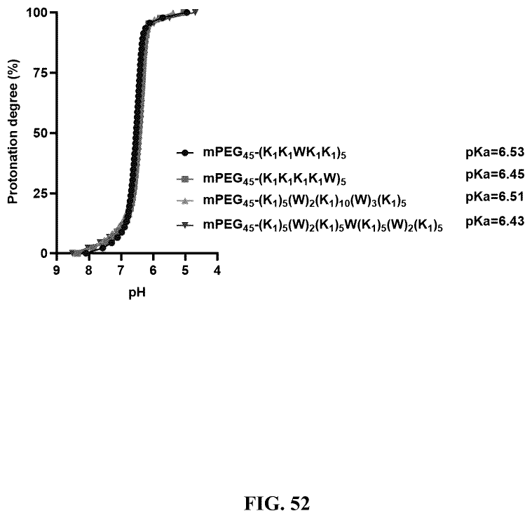

100 μL of concentrated hydrochloric acid (3700, 12 mol) was added to 100 mL of the deionized water and fully mixed to obtain a clear and transparent solution. 10 mg of the sequence-defined polypeptide material prepared in step (5) was dissolved in 10 mL of the hydrochloric acid solution, and a pH probe was inserted immersed below a liquid surface. Under a stirring condition (a stirring speed Mot of 3), titration was performed using a 0.5 M sodium hydroxide titrant. After a pH meter stabilized, a reading was recorded. The titration continued until the reading reached pH=11. A derivative of a titration curve was taken to obtain extremum points, which were defined as points of protonation degrees of 1 and 0, respectively, thereby establishing a pH-protonation degree curve. According to a definition of a dissociation equilibrium constant (pKa), a pH corresponding to a protonation degree of 0.5 for the polypeptide was defined as a pKa of the material. The results are shown in FIG. 52, indicating that different arrangements of the sequences have little effect on protonation degree of the polypeptides.

### (III) Cytotoxicity of Sequence-Defined Polypeptides at Different pH Values

10 mg of the polypeptide prepared in step (5) was placed in a 5 mL sample vial, and 0.3 mL of DMF was added for fully dissolution for later use. 0.9 mL of sterile water was added in a pre-sterilized 25 mL round-bottom flask on a magnetic stirrer and stirred at a speed of RPM=600 r/min. A DMF solution of the polypeptide was added dropwise to the round-bottom flask using a pipette. Stirring was continued at a speed of RPM=400 r/min for 15 min. Dialysis was then performed using a dialysis bag with a molecular weight cut-off of 14000 Da in 4 μL of ultrapure water at 4° C. for 24 h. The ultrapure water was replaced every 1 h during first 6 h and every 6 h for subsequent 18 h, yielding polypeptide nanoparticles. After the dialysis, the solution was taken out using the pipette, quantified, and stored in a 4° C. refrigerator.

A killing effect of the polypeptide nanoparticles on tumor cells under pH 7.4 and pH 6.8 conditions was evaluated by a CCK-8 assay. A DMEM was adjusted to pH=6.8 with a 6 mol/L hydrochloric acid solution and reserved for later use. Mouse colorectal cancer cells MC38 (purchased from ATCC) were cultured in a DMEM containing 10% (v/v) fetal bovine serum. Under pH 7.4 and pH 6.8 conditions, cells (at a concentration of 1×10⁵ cells/mL) were incubated with different concentrations of the polypeptide nanoparticles prepared in this example. After incubation for 24 h in a 37° C. incubator, the original medium was discarded. 100 μL of a CCK-8 solution was added, and incubation continued in a 37° C., CO₂ incubator for 2 h. An absorbance at 450 nm was measured using a microplate reader.

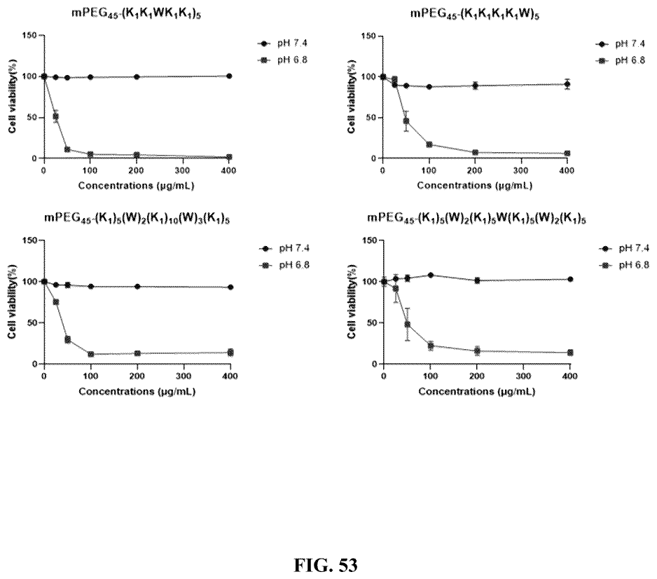

The test results are shown in FIG. 53. The results indicate that different arrangements (sequences) of lysine and tryptophan have little effect on cytotoxicity and selectivity of the polypeptides.

### Example 5: Sequence-Defined Polypeptide Of Tertiary Amine-Modified methoxy poly(ethylene glycol)-lysine and phenylalanine

R—OH was 2-(dibutylamino)ethanol (DB) or (S)-3-dimethylamino-3-phenylpropanol (DMP2), R₂ was a phenylalanine residue, and y was 45. mPEG₄₅-COOH was reacted with an amino group in (K)ₙ(F)ₘ sequence-defined polypeptide to obtain mPEG₄₅-(K)ₙ(F)ₘ. A tertiary amine was modified onto a side chain of lysine via CDI to obtain a tertiary amine-modified sequence-defined polypeptide. An effect of replacing a hydrophobic amino acid with phenylalanine on a pKa and pH-selective killing of the sequence-defined polypeptide was tested.

### (I) Preparation of Tertiary Amine-Modified Sequence-Defined Polypeptides

A reaction scheme is as follows:

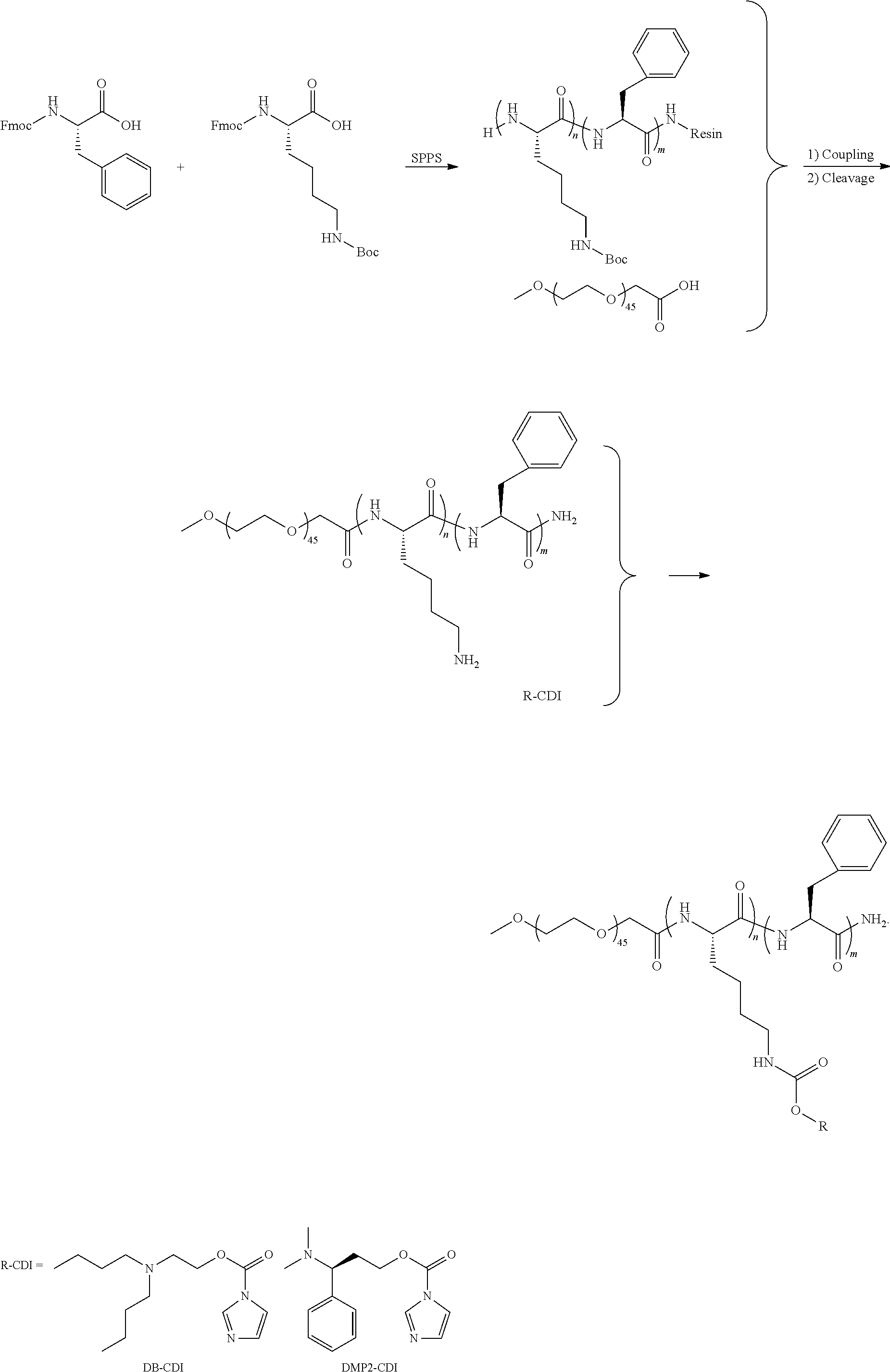

Specific steps were as follows:

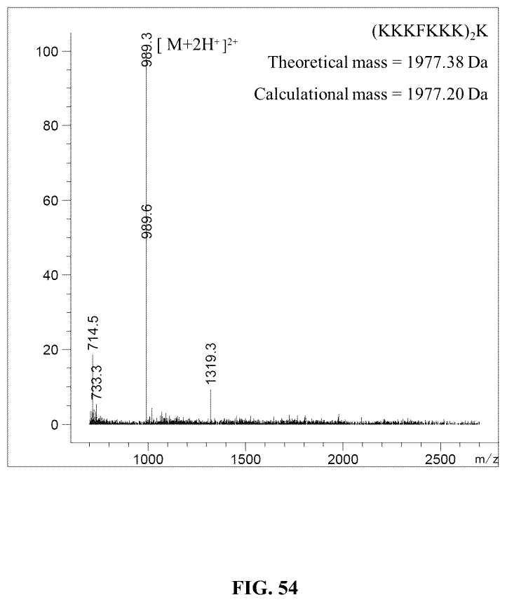

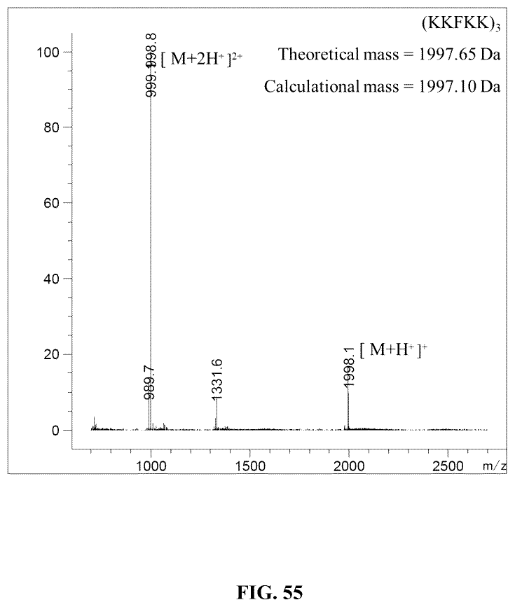

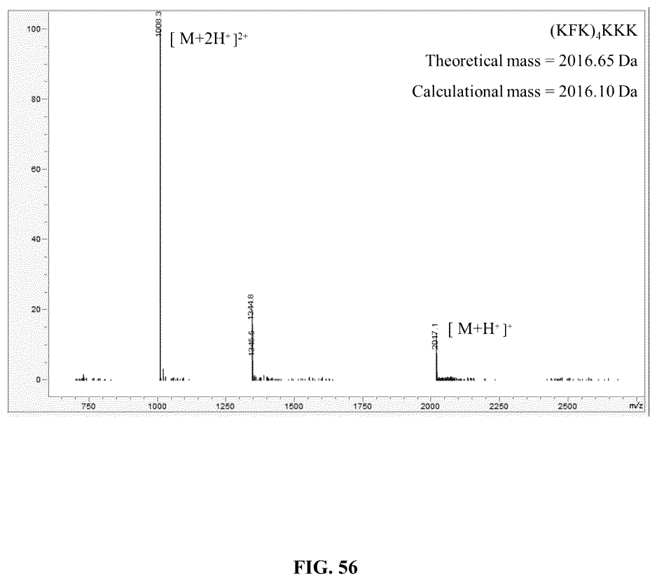

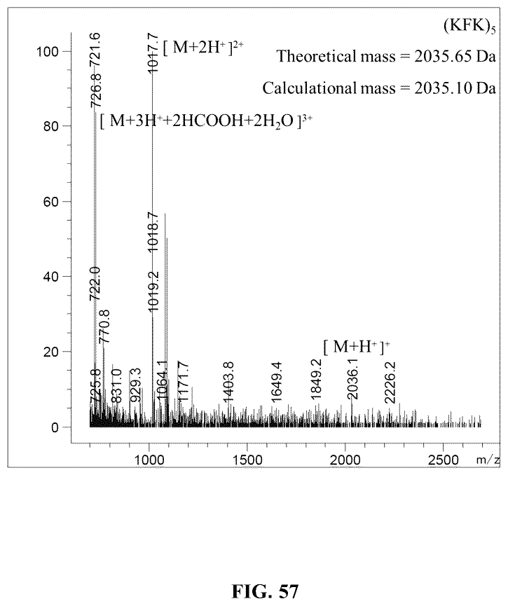

(1) H-[Lys(Boc)]ₙFₘ-Resin was synthesized by SPPS. After each amino acid coupling, a completeness of an amino group reaction was tested using a ninhydrin reagent. A portion of the resin was cleaved using a cleavage solution prepared from 95% TFA, 2.5% H₂O, and 2.5% TIS. After the cleavage, the product was characterized by mass spectrometry. The results are shown in FIG. 54-FIG. 57, indicating that polypeptides with sequences of KKKFKKKKKKFKKKK, KKFKKKKFKKKKFKK, KFKKFKKFKKFKKKK, KFKKFKKFKKFKKFK, KFKFKFKFKFKFKFK, and KFFKKFFKKFFKKKF are obtained.

(2) mPEG₄₅-[Lys(Boc)]ₙFₘ-Resin was synthesized by the SPPS. After the reaction was complete, the ninhydrin reagent was used to detect whether the amino group reaction was complete.

(3) mPEG₄₅-[Lys(Boc)]ₙFₘ sequence-defined polypeptide was cleaved for 3 h using the cleavage solution prepared from 95% TFA, 2.5% H₂O, and 2.5% TIS. The cleavage solution was concentrated with nitrogen gas and then precipitated into ice-cold ether. The precipitate was obtained by centrifugation, dissolved in deionized water, loaded into a 3500 Da dialysis bag, and dialyzed in the deionized water for 24 h, and the deionized water was replaced every 2 h. After lyophilization, a white powder of mPEG₄₅-(K)ₙ(F)ₘ sequence-defined polypeptide was obtained. Specific sequences are mPEG₄₅-KKKFKKKKKKFKKKK, mPEG₄₅-KKFKKKKFKKKKFKK, mPEG₄₅-KFKKFKKFKKFKKKK, mPEG₄₅-KFKKFKKFKKFKKFK, mPEG₄₅-KFKFKFKFKFKFKFK, mPEG₄₅-KFFKKFFKKFFKKKF, which are referred to as mPEG₄₅-(KKKFKKK)₂K, mPEG₄₅-(KKFKK)₃, mPEG₄₅-(KFK)₄KKK, mPEG₄₅-(KFK)₅, mPEG₄₅-(KF)₇K, and mPEG₄₅-(KFFK)₃KKF, respectively.

(4) The CDI was placed in a round-bottom flask, and anhydrous dichloromethane was added for stirring and dispersion (5 mL dichloromethane for 1.0 g CDI). The round-bottom flask was sealed with a rubber stopper. R—OH (3-fold excess relative to the CDI) was slowly added using a syringe. The solution gradually became clear. After reacting for 12 h, an equal volume of the deionized water relative to the dichloromethane was added. Stirring was continued until no bubbles emerged. A lower dichloromethane phase was collected using a separatory funnel. Anhydrous magnesium sulfate was added for drying for 2 h. The solid was removed by filtration to obtain a solution, which was then concentrated under reduced pressure and dried to obtain R-CDI.

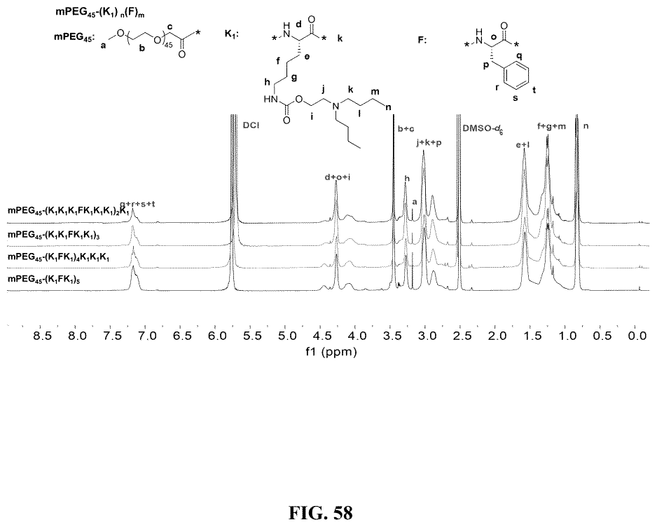

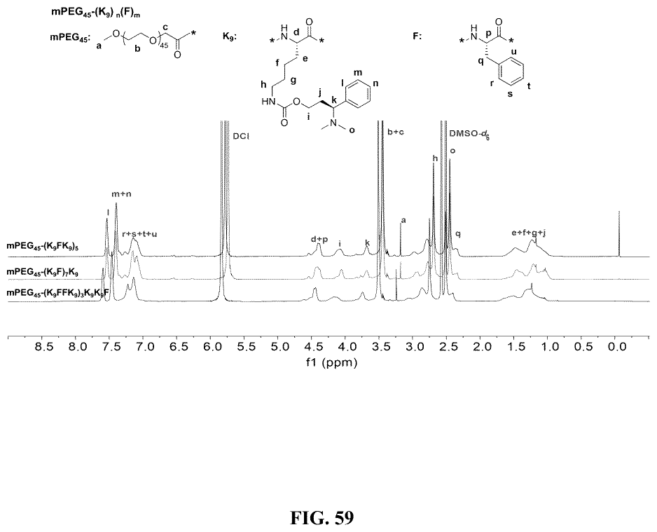

(5) The mPEG₂ₖ-(K)ₙ(F)ₘ sequence-defined polypeptide was dissolved in DMF. The R-CDI (5-fold excess) and triethylamine (an equimolar to the lysine side chain amino groups). The reaction proceeded for 12 h. The solvent was removed using an oil pump. The crude product was dissolved in DMSO, loaded into a 3.5 kDa dialysis bag, and dialyzed in the deionized water for 24 h, and the deionized water was replaced every 2 h. After lyophilization, sequence-defined polypeptide modified with DB or DMP2 was obtained. The DB-modified lysine is denoted as K₁, and the DMP2-modified lysine is denoted as K₉. Accordingly, the sequence-defined polypeptides obtained in this example are referred to as mPEG₄₅-(K₁K₁K₁FK₁K₁K₁)₂K₁, mPEG₄₅-(K₁K₁FK₁K₁)₃, mPEG₄₅-(K₁FK₁)₄K₁K₁K₁, mPEG₄₅-(K₁FK₁)₅, mPEG₄₅-(K₉FK₉)₅, mPEG₄₅-(K₉F)₇K₉, and mPEG₄₅-(K₉FFK₉)₃K₉K₉F. Structures were characterized by proton nuclear magnetic resonance (¹H NMR) spectra (as shown in FIG. 58-FIG. 59). For the DB-modified lysine, a peak of a methylene of the lysine side chain connected to a primary amine group disappears, proving complete bonding. For the DMP2-modified lysine, 1+m+n:q=K:F, proving complete bonding.

Structural formulas of the sequence-defined polypeptides prepared in this example are as follows:

| | |
|---|---|
| mPEG₄₅-(K₁K₁K₁FK₁K₁K₁)₂K₁ | mPEG₄₅-(K₁K₁FK₁K₁)₃ |
| 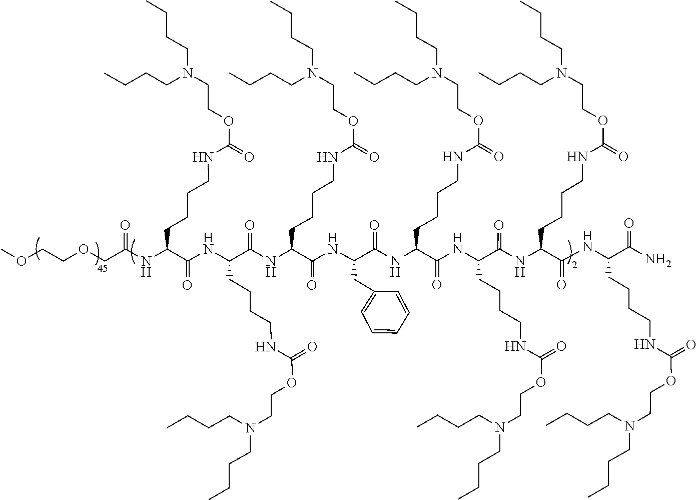 | 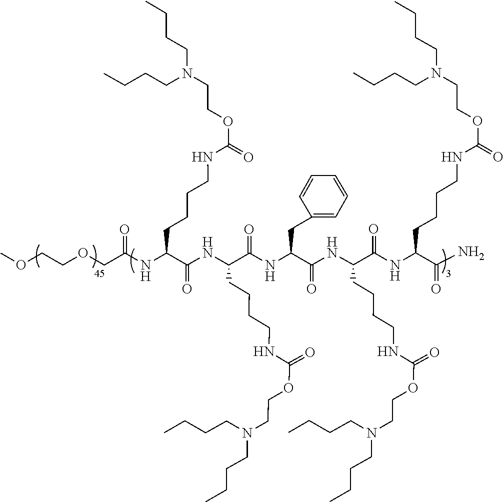 |
| mPEG₄₅-(K₁FK₁)₄K₁K₁K₁ | mPEG₄₅-(K₁FK₁)₅ |
| 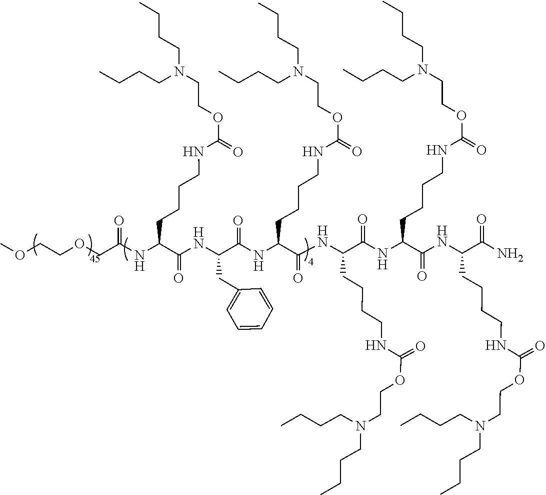 | 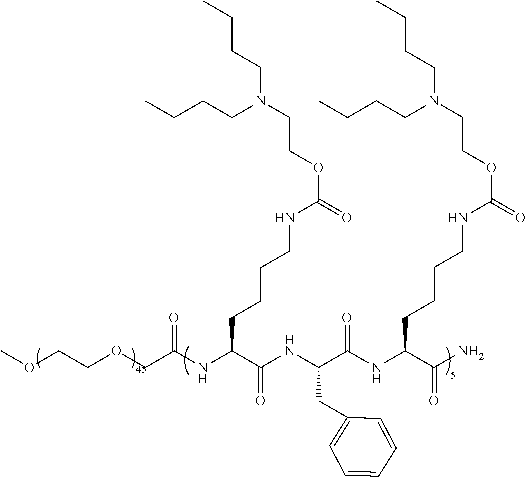 |
| mPEG₄₅-(K₉FK₉)₅ | mPEG₄₅-(K₉F)₇K₉ |
| 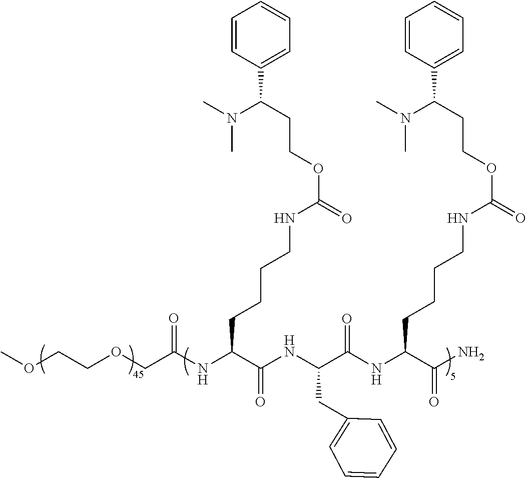 | 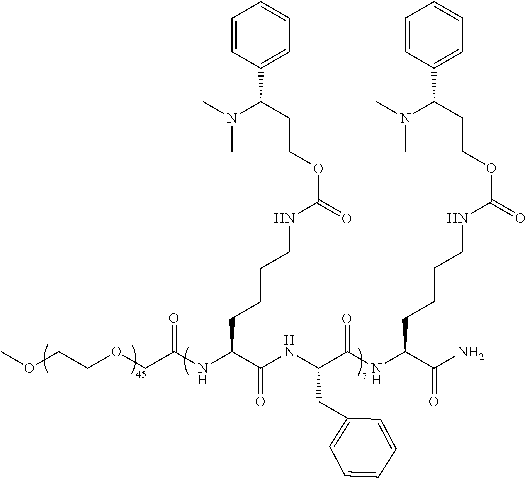 |
| mPEG₄₅-(K₉FFK₉)₃K₉K₉F | |
| 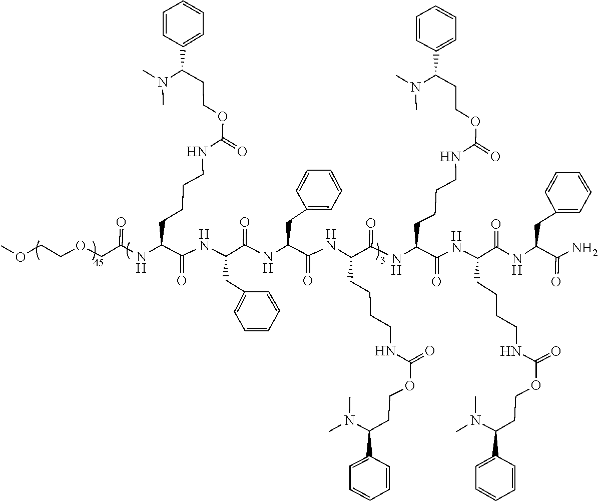 | |

### (II) Effects of Tertiary Amine Modification on Lysine Side Chains and Different Hydrophobic Amino Acids on pKa of Polypeptides

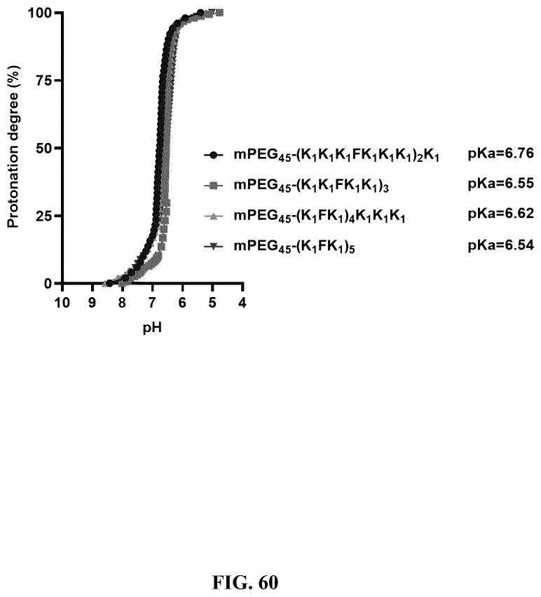

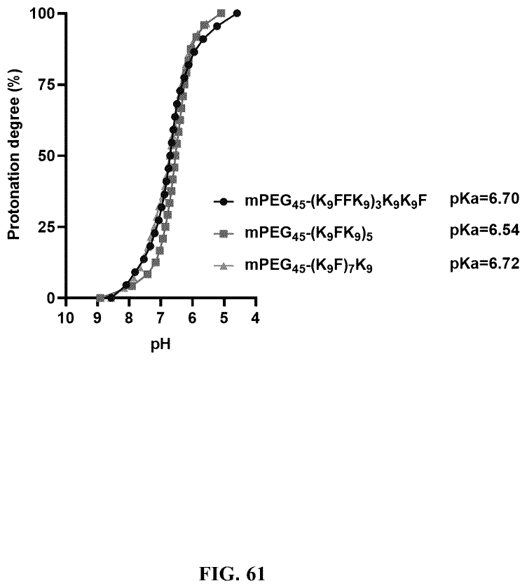

100 μL of concentrated hydrochloric acid (37%, 12 mol) was added to 100 mL of the deionized water and fully mixed to obtain a clear and transparent solution. 10 mg of the sequence-defined polypeptide material prepared in step (5) was dissolved in 10 mL of the hydrochloric acid solution. A pH probe was immersed below a liquid surface. Under a stirring condition (a stirring speed Mot of 3), a titration was performed using a 0.5 M sodium hydroxide titrant. After a pH meter stabilized, a reading was recorded. The titration continued until the reading reached pH=11. A derivative of a titration curve was taken to obtain extremum points, which were defined as points of protonation degrees of 1 and 0, respectively, thereby establishing a pH-protonation degree curve. According to a definition of a dissociation equilibrium constant (pKa), a pH corresponding to a protonation degree of 0.5 for the polypeptide was defined as a pKa of the material. The results are shown in FIG. 60-FIG. 61, indicating the protonation degree of this type of polypeptides exhibits a sharp transition with changing pH.

### (3) Cytotoxicity of Sequence-Defined Polypeptides at Different pH Values

10 mg of the polypeptide prepared in step (5) was placed in a 5 mL sample vial, and 0.3 mL of DMF was added for fully dissolution for later use. 0.9 mL of sterile water was added into a pre-sterilized 25 mL round-bottom flask on a magnetic stirrer. The solution was stirred at a speed of RPM=600 r/min. A DMF solution of the polypeptide was added dropwise to the round-bottom flask using a pipette. Stirring was continued at RPM=400 r/min for 15 min. Dialysis was then performed using a dialysis bag with a molecular weight cut-off of 14000 Da in 4 μL of ultrapure water at 4° C. for 24 h. The ultrapure water was replaced every 1 h during first 6 h and every 6 h for subsequent 18 h, yielding polypeptide nanoparticles. After the dialysis, the solution was taken out using the pipette, quantified, and stored in a 4° C. refrigerator.

A killing effect of the polypeptide nanoparticles on tumor cells under pH 7.4 and pH 6.8 conditions was evaluated by a CCK-8 assay. A DMEM was adjusted to pH=6.8 with a 6 mol/L hydrochloric acid solution and reserved for later use. Mouse colorectal cancer cells MC38 (purchased from ATCC) were cultured in a DMEM containing 10% (v/v) fetal bovine serum. Under pH 7.4 and pH 6.8 conditions, cells (at a concentration of 1×10′ cells/mL) were incubated with different concentrations of the polypeptide nanoparticles prepared in this example. After incubation for 24 h in a 37° C. incubator, the original medium was discarded. 100 μL of a CCK-8 solution was added, and incubation continued in a 37° C., CO₂ incubator for 2 h. An absorbance at 450 nm was measured using a microplate reader.

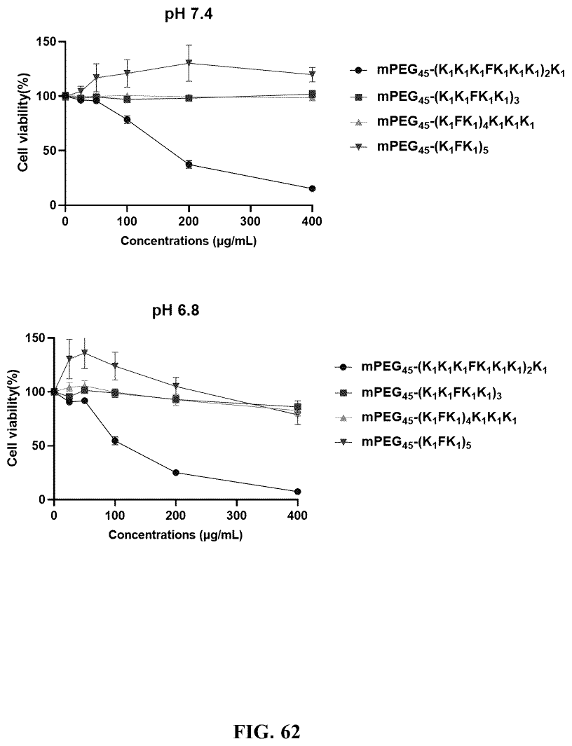

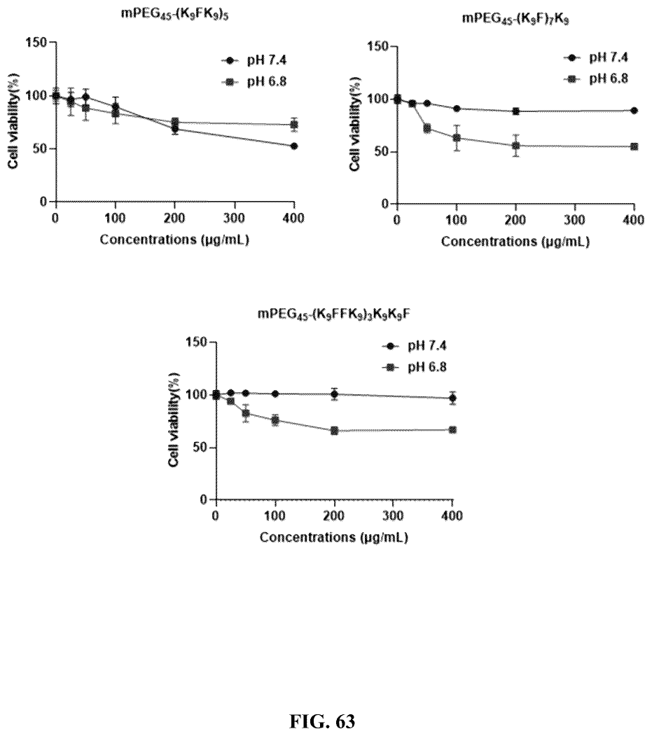

The test results are shown in FIG. 62-FIG. 63. For sequence-defined polypeptides of DB-modified lysine and phenylalanine, as a proportion of phenylalanine increases, cytotoxicity of the polypeptide to the tumor cells decreases. However, none of the polypeptides exhibits pH-selective killing. For sequence-defined polypeptides of DMP2-modified lysine and phenylalanine, as the proportion of phenylalanine increases, cytotoxicity of the polypeptides at pH 7.4 decreases. However, cytotoxicity of the three DMP2-modified polypeptides at pH 6.8 is relatively weak.

The basic concepts have been described above. Obviously, to those skilled in the art, the detailed disclosure above is merely by way of example and does not constitute a limitation to the present disclosure. Although not explicitly stated herein, those skilled in the art may make various modifications, improvements, and amendments to the present disclosure. Such modifications, improvements, and amendments are suggested in the present disclosure. Therefore, such modifications, improvements, and amendments still fall within the spirit and scope of the exemplary embodiments of the present disclosure.

Meanwhile, the present disclosure uses specific words to describe the embodiments of the present disclosure. For example, terms “an embodiment,” “one embodiment,” and/or “some embodiments” mean a certain feature, structure, or characteristic related to at least one embodiment of the present disclosure. Therefore, it should be emphasized and noted that the terms “an embodiment” or “one embodiment” or “an alternative embodiment” mentioned two or more times in different places in the present disclosure do not necessarily refer to the same embodiment. Furthermore, certain features, structures, or characteristics in one or more embodiments of the present disclosure may be appropriately combined.

Furthermore, unless explicitly stated in the claims, the order of processing elements and sequences, the use of numbers and letters, or the use of other names in the present disclosure are not intended to limit the order of the processes and methods of the present disclosure. Although the foregoing disclosure discusses some inventive embodiments currently considered useful through various embodiments, it should be understood that such details are for illustrative purposes only. The appended claims are not limited to the disclosed embodiments. On the contrary, the claims are intended to cover all modifications and equivalent combinations that conform to the essence and scope of the embodiments of the present disclosure.

Similarly, it should be noted that, in order to simplify the expression disclosed in the present disclosure and thereby aid in the understanding of one or more inventive embodiments, the description of the embodiments of the present disclosure above sometimes combines various features into one embodiment, drawing, or description thereof. However, this method of disclosure does not imply that the object of the present disclosure requires more features than those mentioned in the claims. Rather, claimed subject matter may lie in less than all features of a single foregoing disclosed embodiment.

In some embodiments, numbers describing the quantity of ingredients or properties are used. It should be understood that such numbers used in the description of the embodiments are modified by the terms “about,” “approximately,” or “substantially” in some embodiments. Unless otherwise stated, “about,” “approximately,” or “substantially” indicates that the stated number allows a variation of 20%. Accordingly, in some embodiments, the numerical parameters used in the specification and claims are approximate values. These approximate values may vary depending on the desired characteristics of individual embodiments. In some embodiments, numerical parameters should consider the specified number of significant digits and apply the method of general digit retention. Although the numerical ranges and parameters used to confirm the breadth of their scope in some embodiments of the present disclosure are approximate values, in specific embodiments, the setting of such numerical values is as precise as possible within a feasible range.

Each patent, patent application, patent application publication, and other material, such as articles, books, specifications, publications, documents, etc., cited in the present disclosure are hereby incorporated by reference in its entirety. This excludes application history documents that are inconsistent with or conflict with the content of the present disclosure, and also excludes documents that limit the broadest scope of the claims of the present disclosure (whether currently or subsequently appended to the present disclosure). It should be noted that if the description, definition, and/or use of terms in the ancillary materials of the present disclosure are inconsistent with or conflict with the content described in the present disclosure, the description, definition, and/or use of terms in the present disclosure shall prevail.

Finally, it should be understood that the embodiments described in the present disclosure are only used to illustrate the principles of the embodiments of the present disclosure. Other variations may also fall within the scope of the present disclosure. Therefore, by way of example and not limitation, alternative configurations of the embodiments of the present disclosure may be considered consistent with the teachings of the present disclosure. Accordingly, the embodiments of the present disclosure are not limited to the embodiments explicitly introduced and described in the present disclosure.

What is claimed is:

1. A membrane disruptive peptide having a structure as shown in formula (I) or a stereoisomer thereof or a pharmaceutically acceptable salt thereof: 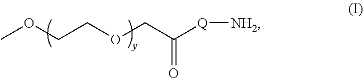
wherein Q is a polypeptide group formed by arranging n A and m B in any sequence;
B is a residue formed by removing one amino hydrogen and one hydroxyl group from an amino acid having a hydrophobic side chain;
A is a residue formed by removing one amino hydrogen and one hydroxyl group from a lysine having a modifying group, and has a structure: 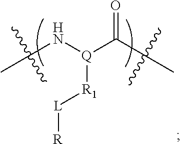
R is selected from the group consisting of: —R₃—N(R₄R₅), —R₃—R′, and 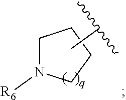
R′ is 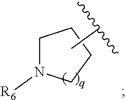
L is selected from the group consisting of: —NH—C(═O)O—, —NH—C(═O)—, —C(═O)—NH—, and —C(═O)—O—;
R₁ is an alkylene;
R₃ is selected from the group consisting of: an alkylene and a C₆-C₁₄ aryl-substituted alkylene;
R₄ and R₅ are each independently selected from the group consisting of: an alkyl, a C₆-C₁₄ aryl-substituted alkyl, or R₃, R₄, and a nitrogen atom to which R₄ and R₅ are connected form a heterocycloalkyl;
R₆ is selected from the group consisting of: a C₁-C₁₅ alkyl, a C₆-C₁₄ aryl, and a C₆-C₁₄ aryl-substituted C₁-C₁₅ alkyl;
y is selected from: 2-150;
n+m is greater than 0, and n is not 0; and
q is selected from: 0, 1, 2, 3, 4.

2. The membrane disruptive peptide or the stereoisomer thereof or the pharmaceutically acceptable salt thereof according to claim 1, wherein the membrane disruptive peptide has a structure as shown in formula (II): 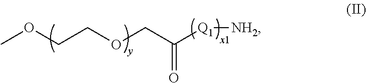
wherein Q₁ is a polypeptide group formed by arranging n₁ A and m₁ B in any sequence;
n₁+m₁ is greater than 0, and n₁ is not 0;
x₁ is greater than 0; and
a product of n₁ and x₁ is n; a product of m₁ and x₁ is m.

3. The membrane disruptive peptide or the stereoisomer thereof or the pharmaceutically acceptable salt thereof according to claim 1, wherein the membrane disruptive peptide has a structure as shown in formula (III): 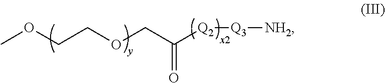
wherein Q₂ is a polypeptide group formed by arranging n₂ A and m₂ B in any sequence;
Q₃ is a polypeptide group formed by arranging n₃ A and m₃ B in any sequence;
n₂+n₃+m₂+m₃ is greater than 0, and n₂+n₃ is not 0; and
a product of n₂ and x₂ plus n₃ is n, and a product of m₂ and x₂ plus m₃ is m.

4. The membrane disruptive peptide or the stereoisomer thereof or the pharmaceutically acceptable salt thereof according to claim 1, wherein B is 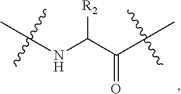
and
R₂ is selected from the group consisting of: a C₁-C₁₂ alkyl, a C₆-C₁₄ aryl, a C₆-C₁₄ aryl-substituted C₁-C₁₂ alkyl, a benzyloxycarbonyl-substituted C₁-C₁₂ alkyl, and a 5-10-membered heteroaryl-substituted C₁-C₁₂ alkyl.

5. The membrane disruptive peptide or the stereoisomer thereof or the pharmaceutically acceptable salt thereof according to claim 1, wherein R₁ is a C₁-C₆ alkylene.

6. The membrane disruptive peptide or the stereoisomer thereof or the pharmaceutically acceptable salt thereof according to claim 1, wherein A is: 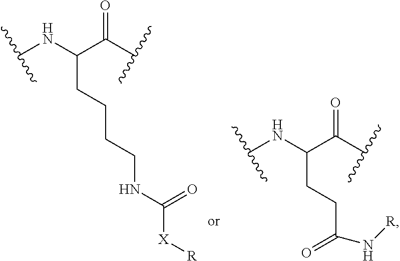
or chiral isomers thereof;
wherein X is: —O— or absent.

7. The membrane disruptive peptide or the stereoisomer thereof or the pharmaceutically acceptable salt thereof according to claim 1, wherein R₃ is selected from the group consisting of: a C₁-C₆ alkylene and a phenyl-substituted C₁-C₆ alkylene.

8. The membrane disruptive peptide or the stereoisomer thereof or the pharmaceutically acceptable salt thereof according to claim 1, wherein R is selected from the group consisting of: —R₃—N(R₄R₅),

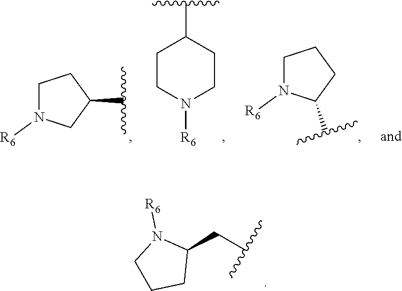

9. The membrane disruptive peptide or the stereoisomer thereof or the pharmaceutically acceptable salt thereof according to claim 1, wherein R₄ and R₅ are each independently selected from the group consisting of: a C₁-C₆ alkyl, a phenyl-substituted C₁-C₆ alkyl, a naphthyl-substituted C₁-C₆ alkyl, or R₄, R₅, and the nitrogen atom to which R₄ and R₅ are connected form a 5-10-membered heterocycloalkyl.

10. The membrane disruptive peptide or the stereoisomer thereof or the pharmaceutically acceptable salt thereof according to claim 9, wherein R₄, R₅, and the nitrogen atom to which R₄ and R₅ are connected form a group selected from:

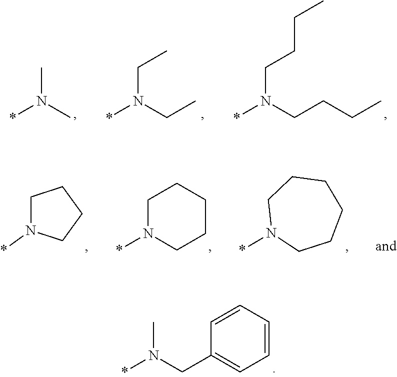

11. The membrane disruptive peptide or the stereoisomer thereof or the pharmaceutically acceptable salt thereof according to claim 1, wherein R₆ is selected from the group consisting of: a C₁-C₆ alkyl, a phenyl, a naphthyl, and a phenyl-substituted C₁-C₆ alkyl.

12. The membrane disruptive peptide or the stereoisomer thereof or the pharmaceutically acceptable salt thereof according to claim 1, wherein R is selected from the group consisting of: —R₃—N(R₄R₅), 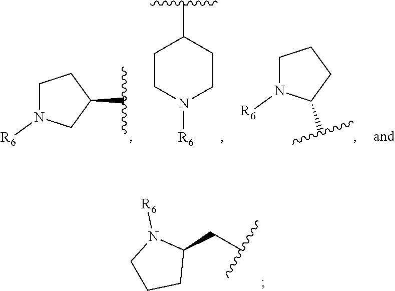
wherein R₃ is selected from the group consisting of —CH₂—CH₂—, —CH₂—CH₂—CH₂—, —CH₂—(CH₂)₃—CH₂—, and 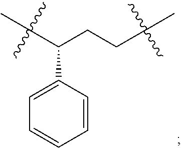 [SMILES: *CC[C@@H](*)c1ccccc1]
R₄, R₅, and the nitrogen atom to which R₄ and R₅ are connected form a group selected from: 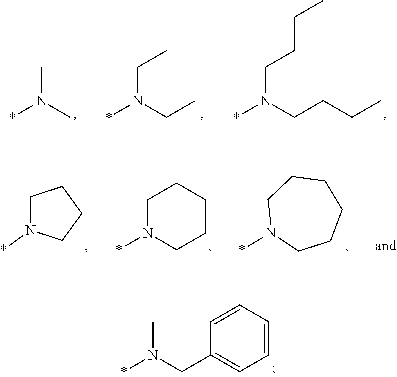
and R₆ is a benzyl.

13. The membrane disruptive peptide or the stereoisomer thereof or the pharmaceutically acceptable salt thereof according to claim 1, wherein y is selected from: 10-120.

14. The membrane disruptive peptide or the stereoisomer thereof or the pharmaceutically acceptable salt thereof according to claim 1, wherein n+m is not less than 5.

15. The membrane disruptive peptide or the stereoisomer thereof or the pharmaceutically acceptable salt thereof according to claim 2, wherein x₁ is selected from 1-30.

16. The membrane disruptive peptide or the stereoisomer thereof or the pharmaceutically acceptable salt thereof according to claim 2, wherein a product of (n₁+m₁) and x₁ is not less than 5.

17. The membrane disruptive peptide or the stereoisomer thereof or the pharmaceutically acceptable salt thereof according to claim 3, wherein x₂ is 1-30.

18. The membrane disruptive peptide or the stereoisomer thereof or the pharmaceutically acceptable salt thereof according to claim 3, wherein a sum of a product of (n₂+m₂) and x₂ plus n₃ and m₃ is not less than 5.

19. The membrane disruptive peptide or the stereoisomer thereof or the pharmaceutically acceptable salt thereof according to claim 1, wherein the membrane disruptive peptide is selected from the group consisting of:

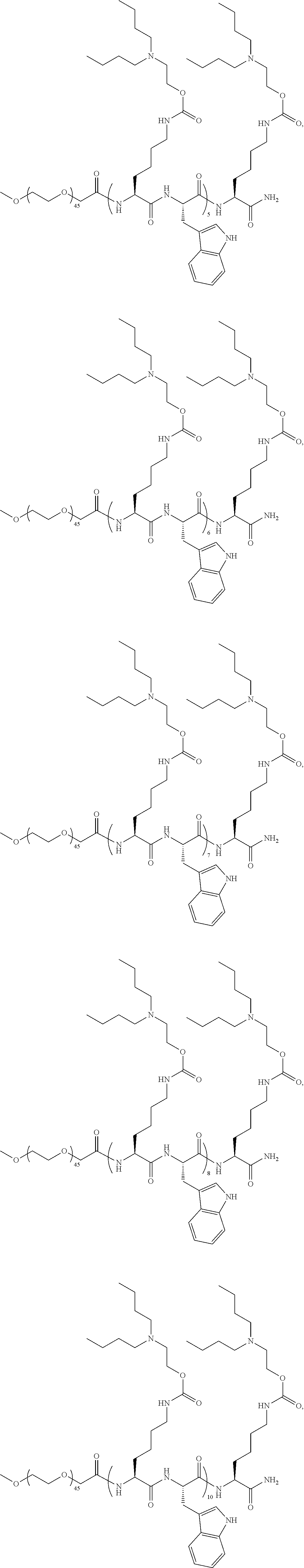

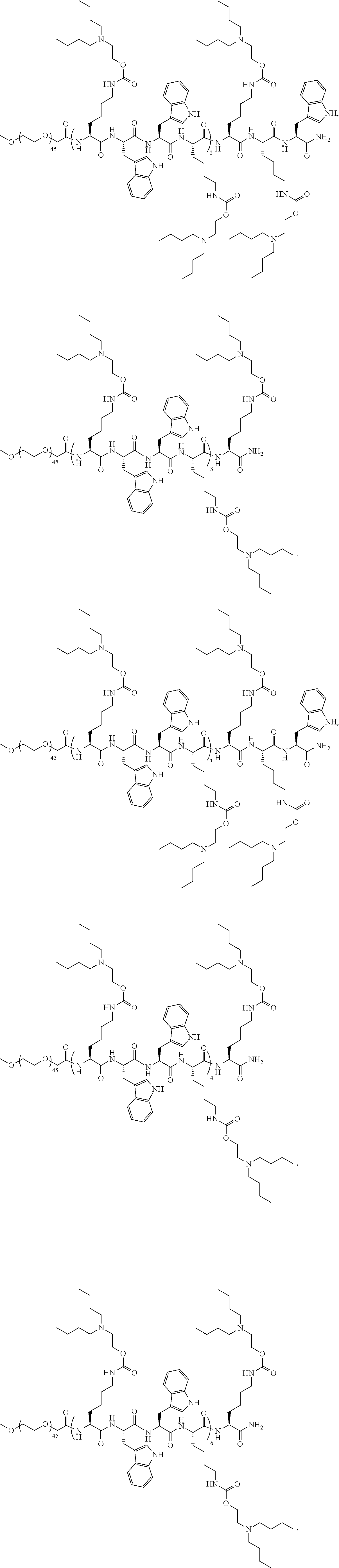

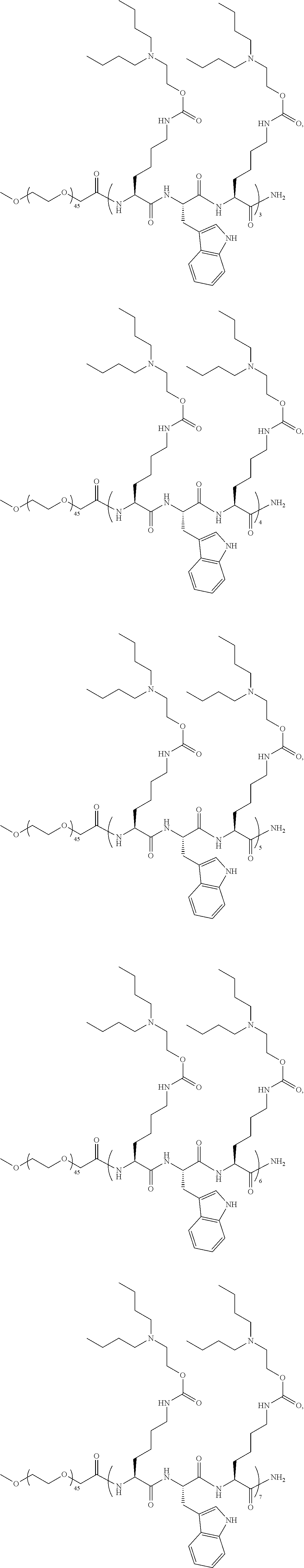

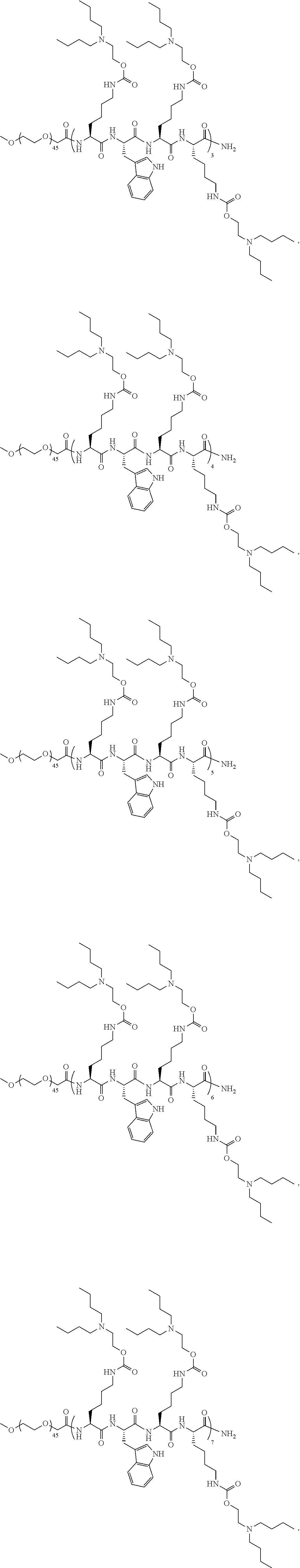

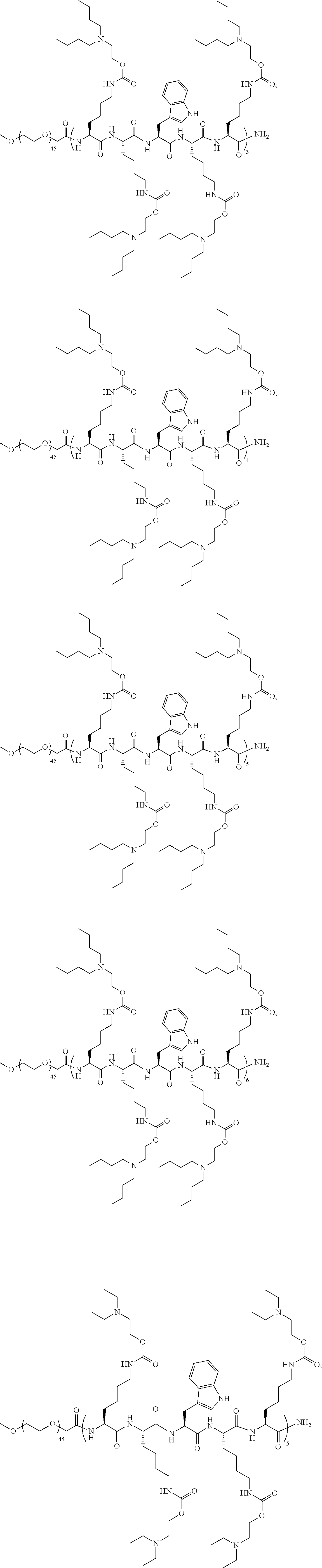

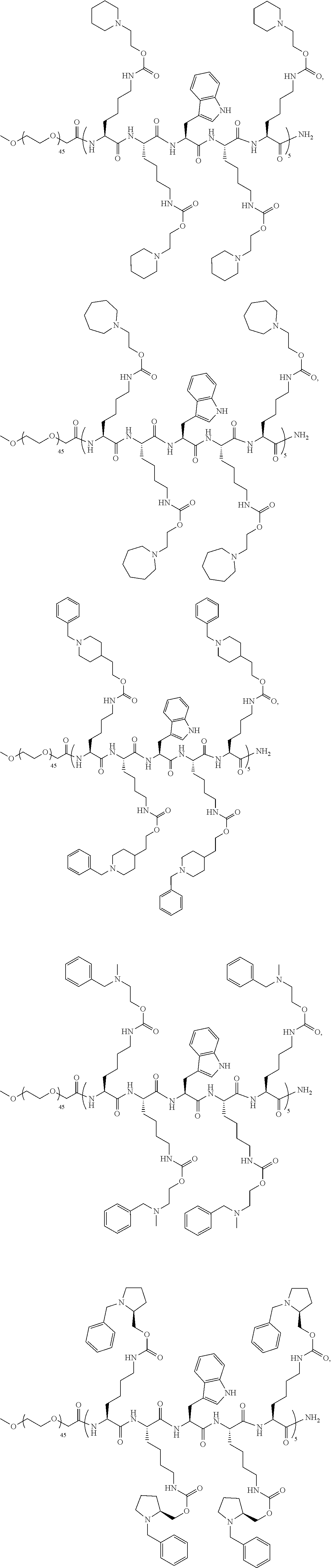

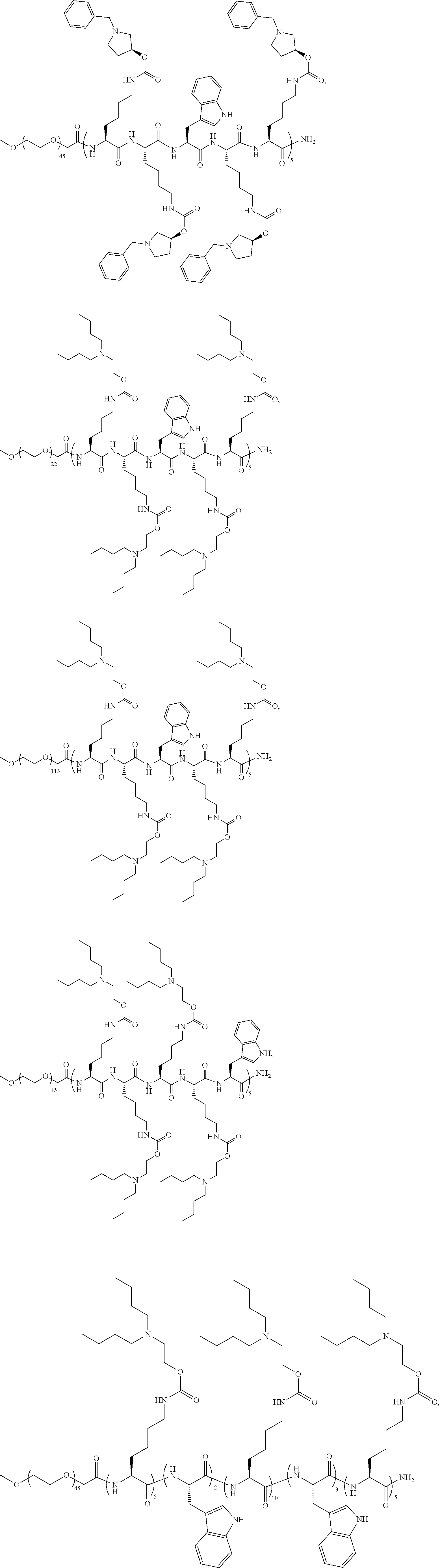

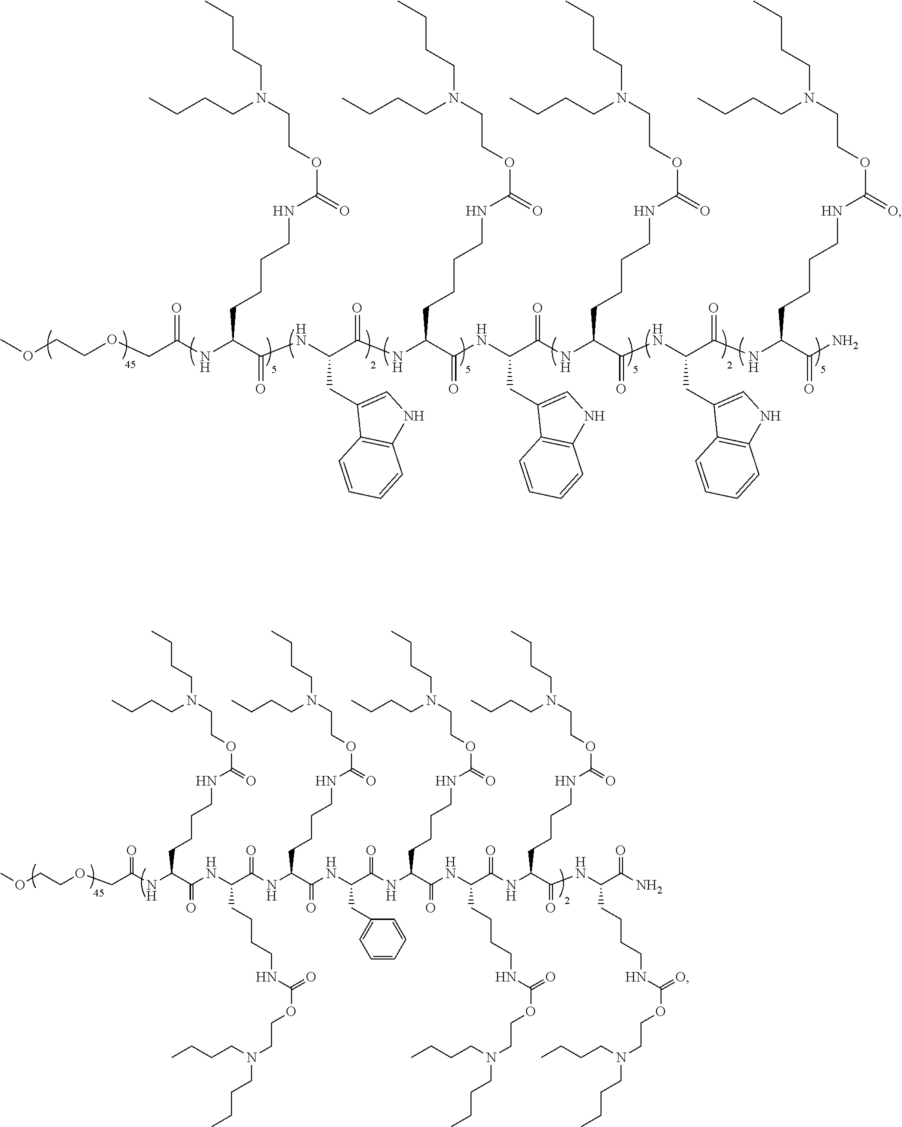

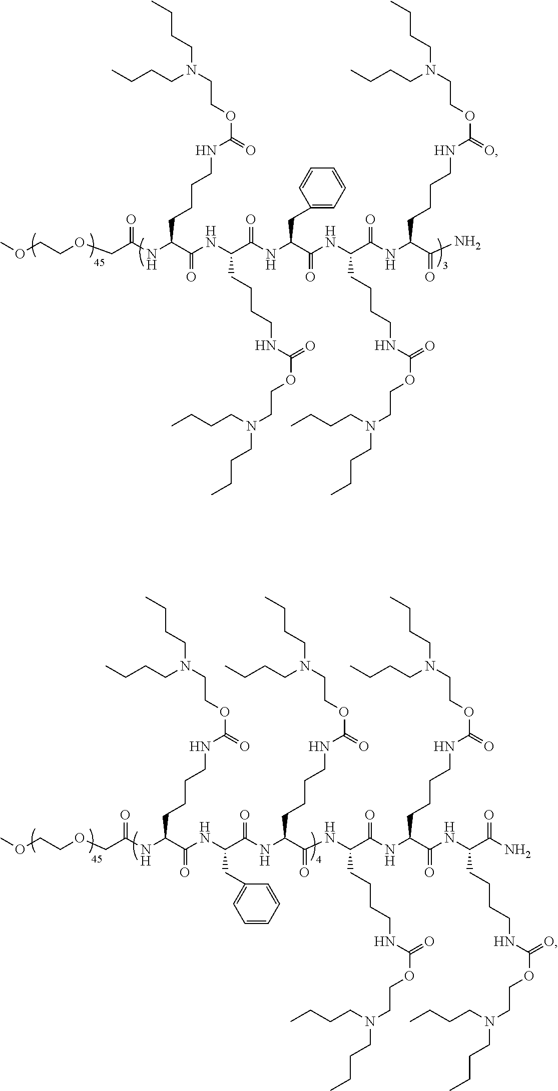

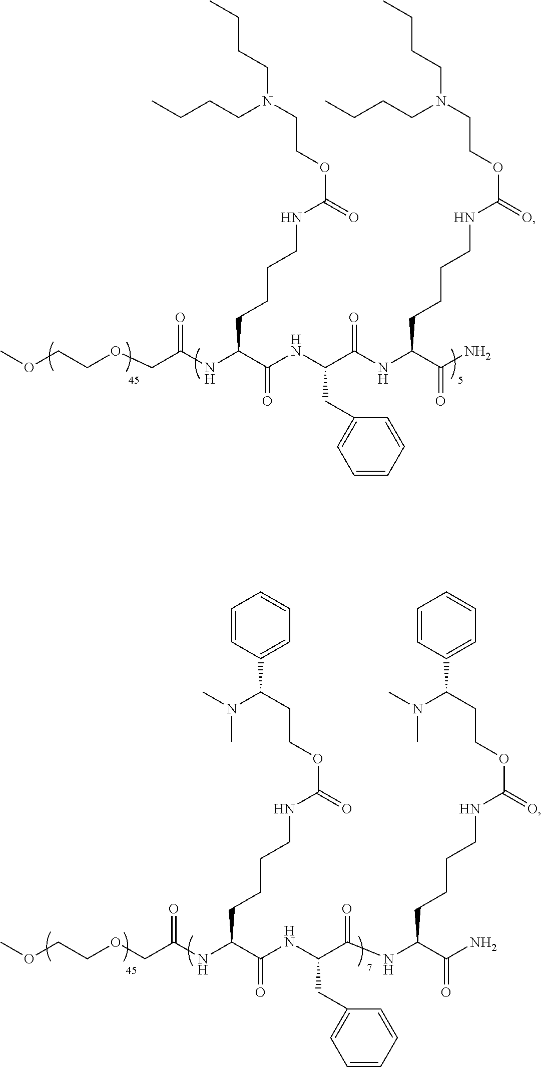

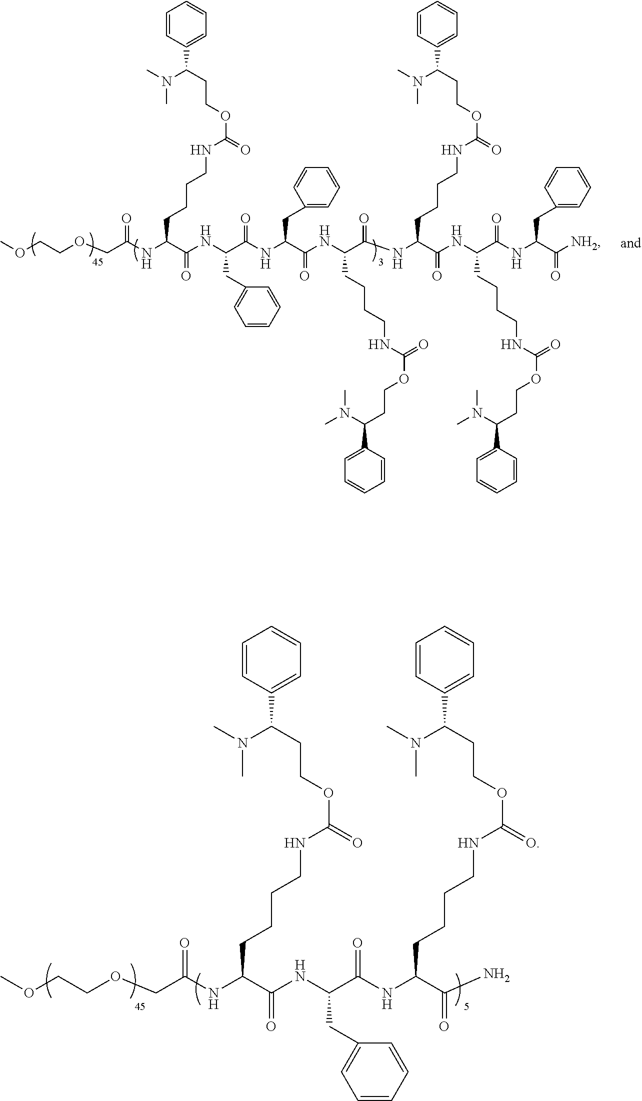

20. A method for preventing and/or treating a tumor, wherein the method comprises: administering a safe and effective amount of the membrane disruptive peptide or the stereoisomer thereof or the pharmaceutically acceptable salt thereof of claim 1 to a patient.
